SEQUENCE LISTING (133 sequences)

SEQ ID NO: 1 - protein, 45 aa, Artificial Sequence
  cartilage-oligomeric matrix protein (COMP) spacer
DLGPQMLRELQETNAALQDVRELLRQQVREITFLKNTVMECDACG

SEQ ID NO: 2 - protein, 7 aa, Artificial Sequence
  variable heavy chain (VH) complementarity determining region (CDR), VH CDR1
GYTFTGY

SEQ ID NO: 3 - protein, 6 aa, Artificial Sequence
  VH CDR2
NPYNDD

SEQ ID NO: 4 - protein, 15 aa, Artificial Sequence
  VH CDR3
GAGYNFDGAYRFFDF

SEQ ID NO: 5 - protein, 16 aa, Artificial Sequence
  variable light chain (VL) CDR, VL CDR1
RSSQRLVHSNGNTYLH

SEQ ID NO: 6 - protein, 7 aa, Artificial Sequence
  VL CDR2
RVSNRFP

SEQ ID NO: 7 - protein, 9 aa, Artificial Sequence
  VL CDR3
SQSTHVPYT

SEQ ID NO: 8 - protein, 124 aa, Artificial Sequence
  hJovi-1 variable heavy chain (VH)
QVQLVQSGAEVKKPGASVKVSCKASGYTFTGYVMHWVRQAPGQGLEWMGFINPYNDDIQSNERFRGRVTMTRDTSISTAYMELSRLRSDDTAVYYCARGAGYNFDGAYRFFDFWGQGTMVTVSS

SEQ ID NO: 9 - protein, 112 aa, Artificial Sequence
  hJovi-1 variable light chain (VL)
DIVMTQSPLSLPVTPGEPASISCRSSQRLVHSNGNTYLHWYLQKPGQSPRLLIYRVSNRFPGVPDRFSGSGSGTDFTLKISRVEAEDVGVYYCSQSTHVPYTFGQGTKLEIK

SEQ ID NO: 10 - protein, 252 aa, Artificial Sequence
  Jovi-1 single-chain variable fragment (scFv)
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSGGGGSGGGGSGGGGSDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 11 - protein, 13 aa, Artificial Sequence
  peptide bridge comprising the 13 membrane proximal amino acids of the extracellular portion of HLA-A2
LRWEPSSNPTIPI

SEQ ID NO: 12 - protein, 23 aa, Artificial Sequence
  CD8 alpha transmembrane domain
IYIWAPLAGTCGVLLLSLVITLY

SEQ ID NO: 13 - protein, 28 aa, Artificial Sequence
  CD28 transmembrane domain
FWVLVVVGGVLACYSLLVTVAFIIFWVR

SEQ ID NO: 14 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 15 - protein, 109 aa, Homo sapiens
SRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 365 aa, Artificial Sequence
  HLA-A01, amino acid sequence of HLA class I
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 17 - protein, 365 aa, Artificial Sequence [sequence omitted]

SEQ ID NO: 18 - protein, 365 aa, Artificial Sequence
  HLA-A-A03, amino acid sequence of HLA class I
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 19 - protein, 362 aa, Artificial Sequence
  HLA-B07, amino acid sequence of HLA class I
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 20 - protein, 362 aa, Artificial Sequence
  HLA-B08, amino acid sequence of HLA class I
MLVMAPRTVLLLLSAALALTETWAGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 21 - protein, 362 aa, Artificial Sequence
  HLA-B44, amino acid sequence of HLA class I
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 22 - protein, 366 aa, Artificial Sequence
  HLA-C01, amino acid sequence of HLA class I
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 23 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 24 - protein, 362 aa, Homo sapiens [sequence omitted]

SEQ ID NO: 25 - protein, 257 aa, Homo sapiens [sequence omitted]

SEQ ID NO: 26 - protein, 249 aa, Homo sapiens
SWKKALRIPGDLRVATVTLMLAMLSSLLAEGRDSPEDFVYQFKGLCYFTNGTERVRLVTRYIYNREEYARFDSDVGVYRAVTPQGRPVAEYWNSQKEVLERTRAELDTVCRHNYEVGYRGILQRRVEPTVTISPSRTEALNHHNLLVCSVTDFYPGQIKVQWFRNDQEETAGVVSTPLIRNGDWTFQILMLEMTPQRGDVYTCHVEHPSLQSPITVEWRAQSESAQSKMLSGVGGFVLGLIFLGLGLII

SEQ ID NO: 27 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 28 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 29 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 31 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 32 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 33 - protein, 5 aa, Artificial Sequence
  linker peptide
SGGGG

SEQ ID NO: 34 - protein, 16 aa, Artificial Sequence
  linker peptide
SGGGGSGGGGSGGGGS

SEQ ID NO: 35 - protein, 347 aa, Artificial Sequence
  polypeptide sequence comprising an ectodomain from HLA-DRalpha, a transmembrane domain and an intracellular CD3-zeta endodomain
METDTLLLWVLLLWVPGSTGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 36 - protein, 389 aa, Artificial Sequence
  polypeptide sequence comprising an ectodomain from HLA-DRalpha, a transmembrane domain, a 41BB endodomain and an intracellular CD3-zeta endodomain.
METDTLLLWVLLLWVPGSTGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 37 - protein, 388 aa, Artificial Sequence
  polypeptide sequence comprising an ectodomain from HLA-DRalpha, a transmembrane domain, a CD28 endodomain and an intracellular CD3-zeta endodomain
METDTLLLWVLLLWVPGSTGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 38 - protein, 256 aa, Artificial Sequence
  polypeptide sequence comprising an ectodomain from CD79alpha, a 41BB domain and an endodomain from CD79
METDTLLLWVLLLWVPGSTGLWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELRKRWQNEKLGLDAGDEYEDENLYEGLNLDDCSMYEDISRGLQGTYQDVGSLNIGDVQLEKP

SEQ ID NO: 39 - protein, 262 aa, Artificial Sequence
  polypeptide sequence comprising an ectodomain from CD79beta, a CD28 domain and an endodomain from CD79
METDTLLLWVLLLWVPGSTGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRSKRSRLLHSDYMNMTPRRPGPTRKHYQPYAPPRDFAAYRSLDKDDSKAGMEEDHTYEGLDIDQTATYEDIVTLRTGEVKWSVGEHPGQE

SEQ ID NO: 40 - protein, 255 aa, Artificial Sequence
  polypeptide sequence comprising an ectodomain from CD79alpha, a CD28 domain and an endodomain from CD79
METDTLLLWVLLLWVPGSTGLWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSRKRWQNEKLGLDAGDEYEDENLYEGLNLDDCSMYEDISRGLQGTYQDVGSLNIGDVQLEKP

SEQ ID NO: 41 - protein, 263 aa, Artificial Sequence
  polypeptide sequence comprising an ectodomain from CD79beta, a 41BB domain and an endodomain from CD79
METDTLLLWVLLLWVPGSTGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLKRGRKKLLYIFKQPFMRPVQTTQEEDGCSCRFPEEEEGGCELLDKDDSKAGMEEDHTYEGLDIDQTATYEDIVTLRTGEVKWSVGEHPGQE

SEQ ID NO: 42 - protein, 308 aa, Artificial Sequence
  polypeptide sequence comprising an ectodomain from CD79alpha, a 41BB domain and a CD3-zeta domain
METDTLLLWVLLLWVPGSTGLWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 43 - protein, 328 aa, Artificial Sequence
  polypeptide sequence comprising an ectodomain from CD79beta, a 41BB domain and a CD3-zeta domain
METDTLLLWVLLLWVPGSTGARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDIITAEGIILLFCAVVPGTLLLFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 44 - protein, 112 aa, Artificial Sequence
  CD3-zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 368 aa, Artificial Sequence
  4-1BB and CD3-zeta endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 152 aa, Artificial Sequence
  CD28 and CD3-zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 188 aa, Artificial Sequence
  CD28, OX40 and CD3-zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 7 aa, Artificial Sequence
  short flexible linker
SGGGSDP

SEQ ID NO: 49 - protein, 6 aa, Artificial Sequence
  serine-glycine linker
SGGGGS

SEQ ID NO: 50 - protein, 100 aa, Artificial Sequence
  CD4 D1 domain
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGL

SEQ ID NO: 51 - protein, 114 aa, Artificial Sequence
  CD8alpha Ig-like V-type domain
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 52 - protein, 5 aa, Artificial Sequence
  peptide spacer repeating unit
GGGGS

SEQ ID NO: 53 - protein, 248 aa, Artificial Sequence
  CD8alpha alpha homodimer
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAGGGGSGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 54 - protein, 111 aa, Artificial Sequence
  CD8beta Ig-like V-type domain
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQL

SEQ ID NO: 55 - protein, 242 aa, Artificial Sequence
  scFv sequence, second binding domain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 56 - protein, 13 aa, Artificial Sequence
  OKT3 heavy chain CDR1
KASGYTFTRYTMH

SEQ ID NO: 57 - protein, 16 aa, Artificial Sequence
  OKT3 heavy chain CDR2
INPSRGYTNYNQKFKD

SEQ ID NO: 58 - protein, 10 aa, Artificial Sequence
  OKT3 heavy chain CDR3
YYDDHYCLDY

SEQ ID NO: 59 - protein, 10 aa, Artificial Sequence
  OKT3 light chain CDR1
SASSSVSYMN

SEQ ID NO: 60 - protein, 11 aa, Artificial Sequence
  OKT3 light chain CDR2
RWIYDTSKLAS

SEQ ID NO: 61 - protein, 9 aa, Artificial Sequence
  OKT3 light chain CDR3
QQWSSNPFT

SEQ ID NO: 62 - protein, 244 aa, Artificial Sequence
  scFv sequence, second binding domain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSSSGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 63 - protein, 26 aa, Artificial Sequence
  spacer sequence, IgG1 hinge
AEPKSPDKTHTCPPCPKDPKSGGGGS

SEQ ID NO: 64 - protein, 45 aa, Artificial Sequence
  spacer sequence, CD8 stalk
TTTPAPRPPTPAPTIASQPLSLRPEACRPAAGGAVHTRGLDFACD

SEQ ID NO: 65 - protein, 521 aa, Artificial Sequence
  calcineurin A, alpha isoform
MSEPKAIDPKLSTTDRVVKAVPFPPSHRLTAKEVFDNDGKPRVDILKAHLMKEGRLEESVALRIITEGASILRQEKNLLDIDAPVTVCGDIHGQFFDLMKLFEVGGSPANTRYLFLGDYVDRGYFSIECVLYLWALKILYPKTLFLLRGNHECRHLTEYFTFKQECKIKYSERVYDACMDAFDCLPLAALMNQQFLCVHGGLSPEINTLDDIRKLDRFKEPPAYGPMCDILWSDPLEDFGNEKTQEHFTHNTVRGCSYFYSYPAVCEFLQHNNLLSILRAHEAQDAGYRMYRKSQTTGFPSLITIFSAPNYLDVYNNKAAVLKYENNVMNIRQFNCSPHPYWLPNFMDVFTWSLPFVGEKVTEMLVNVLNICSDDELGSEEDGFDGATAAARKEVIRNKIRAIGKMARVFSVLREESESVLTLKGLTPTGMLPSGVLSGGKQTLQSATVEAIEADEAIKGFSPQHKITSFEEAKGLDRINERMPPRRDAMPSDANLNSINKALTSETNGTDSNGSNSSNIQ

SEQ ID NO: 66 - protein, 170 aa, Artificial Sequence
  calcineurin B, type 1
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNLKDTQLQQIVDKTIINADKDGDGRISFEEFCAVVGGLDIHKKMVVDV

SEQ ID NO: 67 - protein, 450 aa, Homo sapiens
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 68 - protein, 194 aa, Artificial Sequence
  dominant negative C-terminal Src kinase (dnCSK) sequence, wild-type CSK sequence minus kinase domain (CSK_del_kinase)
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKE

SEQ ID NO: 69 - protein, 132 aa, Artificial Sequence
  dnCSK sequence, wild-type CSK sequence minus kinase and SH3 domains (CSK_del_kinase_SH3)
MSAIQAAWVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKE

SEQ ID NO: 70 - protein, 450 aa, Artificial Sequence
  dnCSK sequence, full length CSK sequence with a K222R substitution (CSK(K222R))
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVRCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 71 - protein, 450 aa, Artificial Sequence
  dnCSK sequence, mutated CSK comprising mutation T266G and SH3 domain deleted
MSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVGEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 72 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 73 - protein, 18 aa, Homo sapiens
MRIFAVFIFMTYWHLLNA

SEQ ID NO: 74 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 75 - protein, 21 aa, Homo sapiens
THLVILGAILLCLGVALTFIF

SEQ ID NO: 76 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 77 - protein, 19 aa, Homo sapiens
MIFLLLMLSLELQLHQIAA

SEQ ID NO: 78 - protein, 200 aa, Homo sapiens
LFTVTVPKELYIIEHGSNVTLECNFDTGSHVNLGAITASLQKVENDTSPHRERATLLEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 79 - protein, 21 aa, Homo sapiens
WLLHIFIPFCIIAFIFIATVI

SEQ ID NO: 80 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 81 - protein, 38 aa, Homo sapiens
MEPPGDWGPPPWRSTPKTDVLRLVLYLTFLGAPCYAPA

SEQ ID NO: 82 - protein, 164 aa, Homo sapiens [sequence omitted]

SEQ ID NO: 83 - protein, 20 aa, Homo sapiens
WWFLSGSLVIVIVCSTVGLI

SEQ ID NO: 84 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 85 - protein, 20 aa, Homo sapiens
MARAMAAAWPLLLVALLVLS

SEQ ID NO: 86 - protein, 323 aa, Homo sapiens
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 87 - protein, 24 aa, Homo sapiens
AIIFLVLGILVFLILLGIGIYFYW

SEQ ID NO: 88 - protein, 431 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 89 - protein, 22 aa, Homo sapiens
MTSQRSPLAPLLLLSLHGVAAS

SEQ ID NO: 90 - protein, 219 aa, Homo sapiens
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 91 - protein, 21 aa, Homo sapiens
LIAGAIGTGAVIIIFCIALIL

SEQ ID NO: 92 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 93 - protein, 16 aa, Homo sapiens
MAFSGSQAPYLSPAVP

SEQ ID NO: 94 - protein, 132 aa, Homo sapiens
FSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQ

SEQ ID NO: 95 - protein, 129 aa, Homo sapiens
FITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 96 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 97 - protein, 24 aa, Homo sapiens
MVVMAPRTLFLLLSGALTLTETWA

SEQ ID NO: 98 - protein, 284 aa, Homo sapiens [sequence omitted]

SEQ ID NO: 99 - protein, 24 aa, Homo sapiens
MGIVAGLVVLAAVVTGAAVAAVLW

SEQ ID NO: 100 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 101 - protein, 34 aa, Homo sapiens
MGHLSAPLHRVRVPWQGLLLTASLLTFWNPPTTA

SEQ ID NO: 102 - protein, 394 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPG

SEQ ID NO: 103 - protein, 23 aa, Homo sapiens
AIAGIVIGVVALVALIAVALACF

SEQ ID NO: 104 - protein, 293 aa, Homo sapiens
MDTTRYSKWGGSSEEVPGGPWGRWVHWSRRPLFLALAVLVTTVLWAVILSILLSKASTERAALLDGHDLLRTNASKQTAALGALKEEVGDCHSCCSGTQAQLQTTRAELGEAQAKLMEQESALRELRERVTQGLAEAGRGREDVRTELFRALEAVRLQNNSCEPCPTSWLSFEGSCYFFSVPKTTWAAAQDHCADASAHLVIVGGLDEQGFLTRNTRGRGYWLGLRAVRHLGKVQGYQWVDGVSLSFSHWNQGEPNDAWGRENCVMMLHTGLWNDAPCDSEKDGWICEKRHNC

SEQ ID NO: 105 - protein, 31 aa, Homo sapiens
MDTTRYSKWGGSSEEVPGGPWGRWVHWSRRP

SEQ ID NO: 106 - protein, 21 aa, Homo sapiens
LFLALAVLVTTVLWAVILSIL

SEQ ID NO: 107 - protein, 241 aa, Homo sapiens
LSKASTERAALLDGHDLLRTNASKQTAALGALKEEVGDCHSCCSGTQAQLQTTRAELGEAQAKLMEQESALRELRERVTQGLAEAGRGREDVRTELFRALEAVRLQNNSCEPCPTSWLSFEGSCYFFSVPKTTWAAAQDHCADASAHLVIVGGLDEQGFLTRNTRGRGYWLGLRAVRHLGKVQGYQWVDGVSLSFSHWNQGEPNDAWGRENCVMMLHTGLWNDAPCDSEKDGWICEKRHNC

SEQ ID NO: 108 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 109 - protein, 22 aa, Homo sapiens
MAKVFSFILVTTALTMGREISA

SEQ ID NO: 110 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 111 - protein, 28 aa, Homo sapiens
MLRRRGSPGMGVHVGAALGALWFCLTGA

SEQ ID NO: 112 - protein, 438 aa, Homo sapiens
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 113 - protein, 21 aa, Homo sapiens
LWVTVGLSVCLIALLVALAFV

SEQ ID NO: 114 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 115 - protein, 24 aa, Homo sapiens
MASLGQILFWSIISIIIILAGAIA

SEQ ID NO: 116 - protein, 235 aa, Homo sapiens
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 117 - protein, 21 aa, Homo sapiens
LCVSSFFAISWALLPLSPYLM

SEQ ID NO: 118 - protein, 243 aa, Artificial Sequence
  CD19 endodomain
QRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTR

SEQ ID NO: 119 - protein, 19 aa, Artificial Sequence
  truncated CD19 endodomain
QRALVLRRKRKRMTDPTRR

SEQ ID NO: 120 - protein, 41 aa, Artificial Sequence
  CD28 endodomain
RSKRSRLLHSDYMNMTPRRPGPTRKHYQPYAPPRDFAAYRS

SEQ ID NO: 121 - protein, 38 aa, Artificial Sequence
  ICOS endodomain
CWLTKKKYSSSVHDPNGEYMFMRAVNTAKKSRLTDVTL

SEQ ID NO: 122 - protein, 41 aa, Artificial Sequence
  CTLA4 endodomain
AVSLSKMLKKRSPLTTGVYVKMPPTEPECEKQFQPYFIPIN

SEQ ID NO: 123 - protein, 42 aa, Artificial Sequence
  41BB endodomain
KRGRKKLLYIFKQPFMRPVQTTQEEDGCSCRFPEEEEGGCEL

SEQ ID NO: 124 - protein, 48 aa, Artificial Sequence
  CD27 endodomain
QRRKYRSNKGESPVEPAEPCHYSCPREEEGSTIPIQEDYRKPEPACSP

SEQ ID NO: 125 - protein, 189 aa, Artificial Sequence
  CD30 endodomain
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 126 - protein, 42 aa, Artificial Sequence
  OX-40 endodomain
ALYLLRRDQRLPPDAHKPPGGGSFRTPIQEEQADAHSTLAKI

SEQ ID NO: 127 - protein, 107 aa, Artificial Sequence
  TACI endodomain
KKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 128 - protein, 58 aa, Artificial Sequence
  GITR endodmain
QLGLHIWQLRSQCMWPRETQLLLEVPPSTEDARSCQFPEEERGERSAEEKGRLGDLWV

SEQ ID NO: 129 - protein, 115 aa, Artificial Sequence
  CD2 endodomain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSS

SEQ ID NO: 130 - protein, 62 aa, Artificial Sequence
  CD40 endodomain
KKVAKKPTNKAPHPKQEPQEINFPDDLPGSNTAAPVQETLHGCQPVTQEDGKESRISVQERQ

SEQ ID NO: 131 - protein, 172 aa, Artificial Sequence
  mutant calcineurin B, CnB30
MGNEASYPLEMCSHFDADEIKRLGKRFKKLDLDNSGSLSVEEFMSLPELQQNPLVQRVIDIFDTDGNGEVDFKEFIEGVSQFSVKGDKEQKLRFAFRIYDMDKDGYISNGELFQVLKMMVGNNTKLADTQLQQIVDKTIINADKDGDGRISFEEFCAVVGGLDIHKKMVVDV

SEQ ID NO: 132 - protein, 20 aa, Artificial Sequence
  cleavage site, 2A-like sequence
RAEGRGSLLTCGDVEENPGP

SEQ ID NO: 133 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

(19) United States
(12) Patent Application Publication
(10) Pub. No.: US 2023/0148144 A1
(43) Pub. Date: May 11, 2023

(54) Cell

(71) Applicant: AUTOLUS LIMITED, London (GB)

(72) Inventors: Shaun Cordoba, London (GB); Martin Pulé, London (GB); Vania Baldan, London (GB); Alex Nicholson, London (GB)

(21) Appl. No.: 17/915,802

(22) PCT Filed: Apr. 8, 2021
(86) PCT No.: PCT/GB2021/050862

§ 371 Date: Sep. 29, 2022

(30) Foreign Application Priority Data
Apr. 8, 2020 (GB) 2005216.3

(51) Int. Cl. A61K 35/17; C12N 9/16; C07K 14/74; C07K 14/725; C12N 5/0783; A61K 31/436; A61K 38/17

(52) U.S. Cl. CPC A61K 35/17; C12N 9/16; C12Y 301/03016; C07K 14/70539; C07K 14/7051; C12N 5/0636; A61K 31/436; A61K 38/1774; C07K 2319/30; C07K 2319/03; A61K 2035/122

(57) ABSTRACT

There is provided an effector immune cell which expresses a cell surface receptor or receptor complex which specifically binds an antigen recognition receptor of a target immune cell; which effector immune cell is engineered such that when a synapse is formed between the effector immune cell and the target immune cell, the capacity of the effector immune cell to kill the target immune cell is greater than the capacity of the target immune cell to kill the effector immune cell. There is also provided the use of such a cell in methods for treating cancer, preventing allograft rejection and GVHD.

### FIELD OF THE INVENTION

The present invention relates to effector immune cells which specifically bind an antigen recognition receptor of a target immune cell and in particular to approaches to control killing of such effector immune cells by the target cells.

### BACKGROUND TO THE INVENTION

### Prevention of Rejection

In solid organ transplants or hematopoietic stem cell transplants (HSCT), mismatches in HLA between recipient and donor can lead to rejection of the organ or graft-vs-host disease (GVHD) respectively. Immunosuppressive drugs can mitigate these outcomes but, due to their broadly inhibitory action against immune cells, they increase the risk of opportunistic infections.

Alloreactive T-cells that recognise mismatches HLA via their T-cell receptor (TCR) are major mediators of rejection and GVHD. CD8+ T cell specificity is dictated by the clonotypic TCR which recognises short antigenic peptides presented on MHC class I molecules. MHC class I molecules are non-covalent heterodimers made up of the membrane-integral, highly polymorphic α-chain and the non-membrane attached non-polymorphic β2 microglobulin (β₂m).

Margalit et al ((2002) International Immunology 15:1379-1387) describe an approach to convert TCR ligands into T-cell activation receptors. They describe T-cells expressing a β2 microglobulin polypeptide which comprises a transmembrane domain and CD3ζ-derived endodomain attached to the C-terminus and an antigenic peptide attached to the N-terminus via a linker. Such cells were found to express a high level of surface peptide-class I complexes and to respond to antibodies and target T-cells in a peptide specific manner. By expressing such a peptide-linker-β2m-TM-CD3ζ polypeptide in T-cells it is possible to specifically target pathogenic CD8=T cells recognising a particular antigenic peptide.

### CAR-T Cells

Traditionally, antigen-specific T-cells have been generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem as transgenic expression of Chimeric Antigen Receptor (CAR) allows generation of large numbers of T-cells specific to any surface antigen by ex vivo viral vector transduction of a bulk population of peripheral blood T-cells.

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals.

The most common forms of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

After infusion, CAR T-cells engraft within the recipient and proliferate after encountering target bearing cells. CAR T-cells then persist and their population slowly contracts over time. CAR T-cell persistence can be determined in clinical studies by real-time PCR for the transgene in blood samples or by flow-cytometry for the CAR in blood samples and clinical researchers have found a correlation between persistence and sustained responses. This correlation is particularly pronounced in CD19 CAR therapy of B-Acute lymphoblastic leukaemia (ALL). Often in this setting, loss of CAR T-cell engraftment heralds relapse of the leukaemia.

CAR T-cells can result in activation of a cellular mediated immune response which can trigger rejection of the CAR T-cells. This is due to immunogenicity of the components engineered into the cell either through non-self proteins or through non-self sequences formed from junctions between self-proteins used to make receptors and other engineering components.

CARs are artificial proteins which are typically composed of a targeting domain, a spacer domain, a transmembrane domain and a signaling domain. The targeting domain is typically derived from an scFv which may be murine. While this scFv can be human or humanized and other components individually are derived from self-proteins, the junctions between them can still be immunogenic. For instance, within the scFv there are junctions between the heavy chain and the linker and the linker and the light chain. There is then a junction between the scFv and the spacer domain.

If the transmembrane domain is not continuous with the spacer there is a further junction there. Similarly, if the transmembrane domain is not continuous with the amino-terminal portion of the endodomain, there is a further junction there. Finally, most endodomains have at least two components and sometimes more with junctions subsequently between each component.

In addition, CAR T-cells are often engineered with further components. These components include suicide genes (e.g. the HSV-TK enzyme). This enzyme was found to be highly immunogenic and caused a cellular immune depletion of CAR T-cells outside of the context of the profound immunosuppression of haploidentical haematopoietic stem cell transplantation. Other less immunogenic suicide genes may still provide some immunogenicity, as almost every kind of engineered component which involves a fusion between two proteins or use of a xenogeneic protein can be immunogenic.

In many settings, CAR T-cells are generated from autologous T-cells. In this setting, allo-responses do not occur. In some circumstances, T-cells from an allogeneic donor are used. This can occur if for instance the patient has had an allogeneic haematopoietic stem cell transplant. In this case, harvested T-cells will be allogeneic. Otherwise, a patient may have insufficient T-cells to generate a CAR T-cell product due to chemotherapy induced lymphopenia.

Rejection of allogeneic cells can be due to minor mismatch or major mismatch. Minor mismatch occurs in the setting where allogeneic T-cells are human leukocyte antigen (HLA)-matched to the recipient. In this case, rejection occurs due to minor histocompatibility antigens which are non-HLA differences between individuals which result in presentation of non-self (donor) epitopes/immunogenic peptides on HLA. In the case where donor and recipient are mismatched, or are only partially matched. T-cell receptors (TCR) on endogenous T-cells of a recipient can interact in a non-specific way with a mismatched HLA and cause rejection consequently. Both minor and major forms of allogeneic rejection are caused by HLA interacting with TCR.

WO2019/073248 and GB application No. 1904971.7 describe an approach which involves coupling the binding of an MHC class I or II on a CAR-expressing cell to a TCR on a T-cell to induce—directly or indirectly—signalling in the CAR-expressing cell. When the CAR-expressing cell is administered to a subject, the MHC class I or II on this cell interacts with any endogenous, reactive T-cells present in the subject through recognition of peptide/MHC complexes. Any such reactive T-cells in the subject are depleted by activation of cytotoxic-mediated cell killing by the CAR-expressing cell.

CAR-mediated approaches to treat T-cell malignancies Lymphoid malignancies can largely be divided into those which are derived from either T-cells or B-cells. T-cell malignancies are a clinically and biologically heterogeneous group of disorders, together comprising 10-20% of non-Hodgkin's lymphomas and 20% of acute leukaemias. The most commonly identified histological subtypes are peripheral T-cell lymphoma, not otherwise specified (PTCL-NOS); angio-immunoblastic T-cell lymphoma (AITL) and anaplastic large cell lymphoma (ALCL). Of all acute Lymphoblastic Leukaemias (ALL), some 20% are of a T-cell phenotype.

These conditions typically behave aggressively, compared for instance with B-cell malignancies, with estimated 5-year survival of only 30%. In the case of T-cell lymphoma, they are associated with a high proportion of patients presenting with disseminated disease, unfavourable International Prognostic Indicator (IPI) score and prevalence of extra-nodal disease. Chemotherapy alone is not usually effective and less than 30% of patients are cured with current treatments.

WO2015/132598 describes a method whereby it is possible to deplete malignant T-cells in a subject, without affecting a significant proportion of healthy T cells. In particular WO2015/132598 describes CARs which specifically bind TCR beta constant region 1 (TRBC1) or TRBC2.

All of the approaches mentioned above, involve the specific binding of a T-cell receptor on a target T-cell. In this situation the targeted T-cell can “fight back” due to ligation of its TCR, resulting in depletion of the grafted/desirable T-cells.

### DESCRIPTION OF THE FIGURES

FIG. 1—(a) MHC class I molecular complex which is composed of MHC and B2M; (b) The TCR complex which is composed of TCRalpha/beta chains surrounded by CD3 elements.

FIG. 2—(a) B2M-Z construct: The B2M construct is fused in frame to a transmembrane domain and CD3-zeta endodomain; (b) B2M-TCR bispecific construct: a scFv which recognizes B2M is fused with a linker to a second scFv which recognizes the CD3/TCR complex. This is then anchored to the membrane via a transmembrane domain; (c) Fusion between B2M and CD3/TCR: As an example, a fusion between B2M via a flexible linker to CD3 Epsilon is shown.

FIG. 3—a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 4—Schematic diagram illustrating the MHC Class I CAR

Major Histocompatibility Complex (MHC) Class I CAR is a heterodimer composed of two non-covalently linked polypeptide chains, α and β2-microglobulin (β2m). The α1 and α2 subunits together with a loaded peptide bind to a T-cell receptor (TCR) expressed on the surface of T cells. β2-microglobulin is connected to a transmembrane domain which anchors the molecule in the cell membrane and is further linked to an endodomain which acts to transmit intracellular signals to the cell. The endodomain can be composed of one or more signalling domains.

FIG. 5—Schematic diagram illustrating three possible β2m-based CAR designs

In the first CAR (A) β2-microglobulin is linked via a bridge to the CD3ζ transmembrane domain which is then linked to the CD3ζ; endodomain. Two other CAR designs (B and C) have added co-stimulatory domains, 41 BB or CD28 respectively.

FIG. 6—(a) A naturally occurring MHC class II molecular complex which is composed of an α chain and a β chain, for example HLA-DRα and HLA-DRβ and presents a peptide; (b) MHC class II molecule comprising an α and a β chain in association with CD79, which comprises CD79α and CD79β which may both contain signalling domains; (c) an engineered MHC class II molecule which comprises an α chain and a β chain wherein the α chain comprises a signalling domain.

FIG. 7—MHC class I and TCR

(a) MHC class I molecules are heterodimers that consist of two polypeptide chains, a and β2-microglobulin (B2M); (b) The TCR complex which is composed of TCRalpha/beta chains surrounded by CD3 elements

FIG. 8—Different MHCIα/TCR fusion constructs

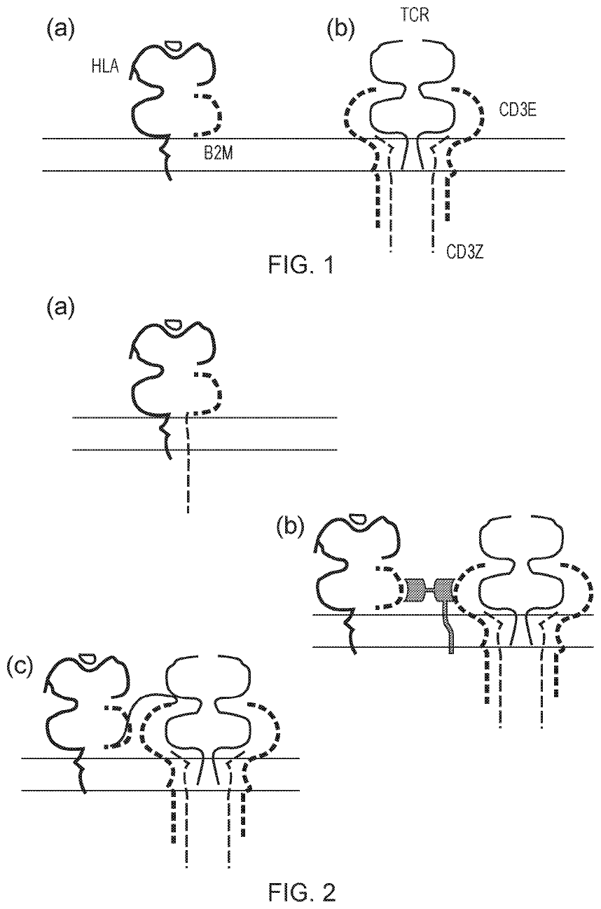

(a) MHCIα-CD3z construct: The MHC class I alpha chain is fused in frame to a TM domain and CD3-zeta endodomain; (b) Ab-CD3z construct: An antibody or antibody-like binder specific to MHC class I alpha chain is fused to a TM domain and CD3-zeta endodomain; (c) Fusion between MHCIα and CD3/TCR: As an example, a fusion between MHC class I alpha chain via a flexible linker to CD3 Epsilon is shown; (d) MHCIα-TCR BiTE construct: a scFv which recognizes MHC class I alpha chain is fused with a linker to a second scFv which recognizes the CD3/TCR complex. This is then anchored to the membrane via a transmembrane domain.

FIG. 9—MHC class II and TCR

(a) MHC class II molecules are heterodimers that consist an α chain and a β chain; (b) The TCR complex which is composed of TCRalpha/beta chains surrounded by CD3 elements

FIG. 10—Different MHCII/TCR fusion constructs

(a) MHCII-CD3z construct: The MHC class II α or β chain is fused to a TM domain and CD3-zeta endodomain; (b) Ab-CD3z construct: An antibody or antibody-like binder specific to MHC class II α or β chain is fused to a TM domain and CD3-zeta endodomain; (c) Fusion between MHCII and CD3/TCR: MHC class I α or β chain is fused via a flexible linker to a component of the TCR/CD3 complex. For example, CD3 Epsilon is shown; (d) MHCII-TCR BiTE construct: a scFv which recognizes MHC class II α or β chain is fused with a linker to a second scFv which recognizes the CD3/TCR complex. This is then anchored to the membrane via a transmembrane domain.

FIG. 11—CD4/CD8 fusion molecules

CD4 and CD8 are TCR co-receptors. The extracellular domain of CD4 binds to the β2 region of MHC class II; whereas the extracellular domain of CD8 binds the α3 portion of the Class I MHC molecule. (a) CD4-CD3z construct: the MHC class II-binding domain of CD4 is fused to a TM domain and CD3-zeta endodomain; (b) CD8-CD3z construct: the MHC class I-binding domain of CD8 is fused to a TM domain and CD3-zeta endodomain

FIG. 12—Data showing killing of cells expressing a truncated version of a TRBC1-specific CAR, lacking a signalling domain, by TRBC1+ target T-cells (reverse killing).

FIG. 13—Data showing persistence of JOVI (or dJOVI) CAR T cells with or without dPDL1 (or dPDL2).

FIG. 14: Schematic diagram illustrating CSK and various dnCSK constructs

A—Wild-type CSK having a SH3 domain, an SH2 domain and a protein tyrosine kinase domain.
B—dnCSK lacking a kinase domain
C—dnCSK lacking a kinase domain and an SH3 domain
6—dnCSK having a mutation K222R.

FIG. 15: Schematic diagram illustrating the mechanism of (a) T-cell activation; and (b) inhibition of T-cell activation by inhibitory immunoreceptors.

FIG. 16: Graphs to show the (A) percentage and (B) number of CAR-expressing (RQR8-positive) cells proliferating after 96 hours co-culture with Jurkat KO, Jurkat TRBC1 and Jurkat TRBC2 target cells, in the absence of Tacrolimus FIG. 17: Graphs to show the (A) percentage and (B) number of CAR-expressing (RQR8-positive) cells proliferating after 96 hours co-culture with Jurkat KO, Jurkat TRBC1 and Jurkat TRBC2 target cells, in the presence of 20 ng/ml of Tacrolimus.

FIG. 18: Graphs to show the number of CAR-expressing (RQR8-positive) cells in each division following co-culture with Jurkat KO, Jurkat TRBC1 and Jurkat TRBC2 target cells, in the absence of Tacrolimus. Proliferation analysis was calculated on single/live/CellTrace Violet-positive cells using FlowJo™ proliferation tool and the CD19 CAR used as the negative control for all the conditions. Cell number in each division is plotted for each CAR+target combination.

FIG. 19: Graphs to show the number of CAR-expressing (RQR8-positive) cells in each division following co-culture with Jurkat KO, Jurkat TRBC1 and Jurkat TRBC2 target cells, in the presence of 20 ng/ml of Tacrolimus. Proliferation analysis was calculated on single/live/CellTrace Violet-positive cells using FlowJo™ proliferation tool and the CD19 CAR used as the negative control for all the conditions. Cell number in each division is plotted for each CAR+target combination.

FIG. 20: Histogram plots showing the proliferation of CAR-expressing (RQR8-positive) cells following co-culture with Jurkat KO, Jurkat TRBC1 and Jurkat TRBC2 target cells, with or without the addition of 20 ng/ml of Tacrolimus. Proliferation analysis was calculated on single/live/CellTrace Violet-positive cells using FlowJo™ proliferation tool and the CD19 CAR used as the negative control for all the conditions. Results are shown using cells from two separate donors.

FIG. 21: Graph showing the cell count of non-transduced cells (NT) and TRBC2 CAR-expressing (RQR8-positive) cells before (day 0) and after (day 4) co-culture with TRBC2 targets with or without the addition of 20 ng/ml of Tacrolimus.

FIG. 22: Graph showing the percentage of TRBC2 CAR-expressing (RQR8-positive) cells before (day 0) and after (day 4) co-culture with TRBC2 targets with or without the addition of 20 ng/ml of Tacrolimus.

FIG. 23: Graph showing killing of TRBC2-expressing PBMCs following co-culture with PBMCs transduced to express: a CD19 CAR, a TRBC2 CAR or to co-express a TRBC2 CAR and a calcineurin mutant module (TRBC2+CnB30). Co-cultures were set up at a 1:1 or a 1:4 E:T ratio in the presence or absence of 20 ng/ml tacrolimus.

FIG. 24: Graph showing survival/proliferation of PBMCs transduced to express: a CD19 CAR, a TRBC2 CAR or to co-express a TRBC2 CAR and a calcineurin mutant module (TRBC2+CnB30) following co-culture with TRBC2-expressing PBMCs. Co-cultures were set up at a 1:1 or a 1:4 E:T ratio in the presence or absence of 20 ng/ml tacrolimus.

FIG. 25: Graph showing IFNγ secretion following co-culture of TRBC2-expressing PBMCs with PBMCs transduced to express: a CD19 CAR, a TRBC2 CAR or to co-express a TRBC2 CAR and a calcineurin mutant module (TRBC2+CnB30). Co-cultures were set up at a 1:1 or a 1:4 E:T ratio in the presence or absence of 20 ng/ml tacrolimus.

FIG. 26: Graph showing IL-2 secretion following co-culture of TRBC2-expressing PBMCs with PBMCs transduced to express: a CD19 CAR, a TRBC2 CAR or to co-express a TRBC2 CAR and a calcineurin mutant module (TRBC2+CnB30). Co-cultures were set up at a 1:1 or a 1:4 E:T ratio in the presence or absence of 20 ng/ml tacrolimus.

### SUMMARY OF ASPECTS OF THE INVENTION

The present inventors have developed approaches for engineering an effector immune cell (cell A) such that, when targeting an autoreactive or pathogenic immune cell (cell B), the engineered immune cell has a selective advantage and the balance between the cell A killing cell B; and cell B killing cell A is tipped in favour of cell A killing cell B.

Thus in a first aspect, the present invention provides an effector immune cell which expresses a cell surface receptor or receptor complex which specifically binds an antigen recognition receptor of a target immune cell; which effector immune cell is engineered such that when a synapse is formed between the effector immune cell and the target immune cell, the capacity of the effector immune cell to kill the target immune cell is greater than the capacity of the target immune cell to kill the effector immune cell.

In a first embodiment of the first aspect of the invention, the effector immune cell is engineered to be resistant to an immunosuppressant.

For example, the effector immune cell may be engineered to be resistant to one or more calcineurin inhibitors.

In this respect, the effector immune cell may express:
calcineurin A comprising mutations T351E and L354A with reference to the shown as SEQ ID No. 65;
calcineurin A comprising mutations V314R and Y341F and with reference to shown as SEQ ID No. 65; or calcineurin B comprising mutation L124T and K-125-LA-Ins with reference to shown as SEQ ID No. 66.

The effector immune cell may be engineered to be resistance to rapamycin.

The effector immune cell may express a dominant negative C-terminal Src kinase (dnCSK), which confers resistance to multiple immunosuppressants.

In a second embodiment of the first aspect of the invention, the effector immune cell is engineered to express or overexpress an immunoinhibitory molecule or a fusion protein comprising the extracellular domain of an immunoinhibitory molecule.

The immunoinhibitory molecule may bind to: PD-1, LAG3, TIM-3, TIGIT, BTLA, VISTA, CEACAM1-R, KIR2DL4, B7-H3 or B7-H4.

The immunoinhibitory molecule may be selected from: PD-L1, PD-L2, HVEM, CD155, VSIG-3, Galectin-9, HLA-G, CEACAM-1, LSECTin, FGL1, B7-H3, and B7-H4.

The effector immune cell may be engineered to express a fusion protein comprising the extracellular domain of an immunoinhibitory molecule and a membrane localisation domain.

The effector immune cell may be engineered to express a fusion protein comprising the extracellular domain of an immunoinhibitory molecule and a co-stimulatory endodomain, such as one selected from CD28, ICOS, CTLA4, 41BB, CD27, CD30, OX-40, TACI, CD2, CD27 and GITR.

The antigen recognition receptor of the target immune cell may, for example, be a T-cell receptor (TCR) or an activating killer cell immunoglobulin-like receptor (KAR).

The cell surface receptor of the effector immune cell may, for example, be a chimeric antigen receptor (CAR) and the antigen recognition receptor is a T-cell receptor (TCR).

Where the effector immune cell expresses a TCR-specific CAR, the CAR may bind TCR beta constant region 1 (TRBC1) or TRBC2.

Alternatively, the cell surface receptor complex of the effector immune cell may be an engineered MHC class I or an engineered MHC class II complex.

For example, the cell surface receptor complex may comprise: an MHC class I polypeptide; an MHC class II polypeptide; or β-2 microglobulin, linked to an intracellular signalling domain.

The cell surface receptor complex may be an engineered MHC class I complex which comprises a molecule having the following structure:

peptide-L-α2M-endo
in which:
“peptide” is a peptide which binds the peptide binding groove of the MHC class I α-chain;
“L” is a linker
“B2M” is β-2 microglobulin; and
“endo” is an intracellular signalling domain.

The effector immune cell may comprise an MHC class I polypeptide: an MHC class II polypeptide; or β-2 microglobulin, linked to a component of the TCR/CD3 complex.

The effector immune cell may comprise an MHC class I polypeptide: an MHC class I polypeptide; an MHC class II polypeptide; or β-2 microglobulin, linked to CD3-zeta, CD3-epsilon, CD3-gamma or CD3-delta via a linker peptide.

The effector immune cell may express a bispecific polypeptide which comprises: (i) a first binding domain which binds an MHC class I polypeptide; an MHC class II polypeptide; or β-2 microglobulin; and (ii) a second binding domain which binds to a component of the TCR/CD3 complex.

The effector immune cell may express an engineered polypeptide which comprises a CD79 α and/or a CD79 β chain linked to an intracellular signalling domain.

The effector immune cell may express an engineered polypeptide which comprises a binding domain which binds to an MHC class I polypeptide or an MHC class II polypeptide, linked to an intracellular signalling domain. The binding domain may be an antibody-like binding domain.

The effector immune cell may express an engineered polypeptide which comprises the MHC class II-binding domain of CD4, or the MHC class I-binding domain of CD8, linked to an intracellular signalling domain.

The effector immune cell of the first aspect of the invention may be engineered to express a cell surface receptor (such as a CAR) or receptor complex (such as an engineered MHC class I or an engineered MHC class II complex) and then further engineered such that when a synapse is formed between the effector immune cell and the target immune cell, the capacity of the effector immune cell to kill the target immune cell is greater than the capacity of the target immune cell to kill the effector immune cell.

The further engineering of the effector immune cell may involve:
(i) engineered the cell to be resistant to an immunosuppressant, or
(ii) engineered the cell to express or overexpress an immunoinhibitory molecule or a fusion protein comprising the extracellular domain of an immunoinhibitory molecule
as described above.

The synapse which is formed between the effector immune cell and the target immune cell is formed when the cell surface receptor or receptor complex of the effector immune cell specifically binds the antigen recognition receptor of the target immune cell.

In a second aspect, there is provided a nucleic acid construct which comprises:
(i) a first nucleic acid sequence which encodes a cell surface receptor or part of a cell surface receptor complex as defined herein; and
(ii) a second nucleic acid sequence which, when expressed in a cell, confers on that cell resistance to an immunosuppressant; and/or
(iii) a third nucleic acid sequence which encodes an immunoinhibitory molecule or a fusion protein comprising the extracellular domain of an immunoinhibitory molecule.

In a third aspect, there is provided a vector comprising a nucleic acid construct according to the second aspect of the invention.

In a fourth aspect there is provided a kit of vectors comprising:
(i) a first vector comprising a nucleic acid sequence which encodes a cell surface receptor or part of a cell surface receptor complex as defined herein; and
(ii) a second vector comprising a nucleic acid sequence which, when expressed in a cell, confers on that cell resistance to an immunosuppressant; and/or
(iii) a third vector comprising a nucleic acid sequence which encodes an immunoinhibitory molecule or a fusion protein comprising the extracellular domain of an immunoinhibitory molecule.

In a fifth aspect, there is provided a pharmaceutical composition comprising a plurality of effector immune cells according to the first aspect of the invention.

In a sixth aspect there is provided a pharmaceutical composition according to the fifth aspect of the invention for use in treating a disease.

In a seventh aspect, there is provided a method for treating a disease, which comprises the step of administering a pharmaceutical composition according to the fifth aspect of the invention to a subject.

The method may comprise the following steps:
(i) administering a pharmaceutical composition to a subject, which pharmaceutical composition comprises a plurality of effector immune cells according to the first aspect of the invention engineered to be resistant to an immunosuppressant; and
(ii) administering the immunosuppressant to the subject.

In an eighth aspect, there is provided the use of a plurality of effector immune cells according to the first aspect of the invention in the manufacture of a medicament for the treatment of a disease.

The disease may be cancer.

In an ninth aspect, there is provided a method for making an effector immune cell according to the first aspect of the invention, which comprises the step of introducing: a nucleic acid construct according to the second aspect of the invention, a vector according to the third aspect of the invention or a kit of vectors according to the fourth aspect of the invention, into the cell ex vivo.

In a tenth aspect, there is provided a method for depleting alloreactive immune cells from a population of immune cells, which comprises the step of contacting the population of immune cells with a plurality of effector immune cells according to the first aspect of the invention wherein the plurality of effector immune cells express an engineered MHC class I or an MHC class II complex as defined herein.

In an eleventh aspect, there is provided a method for treating or preventing graft rejection following allotransplantation, which comprises the step of administering a plurality of effector immune cells derived from the donor subject to the recipient subject for the allotransplant, wherein the plurality of effector immune cells express an engineered MHC class I or an MHC class II complex as defined herein.

In a twelfth aspect, there is provided a method for treating or preventing graft versus host disease (GVHD) associated with allotransplantation, which comprises the step of contacting the allotransplant with administering a plurality of effector immune cells according to the first aspect of the invention, wherein the plurality of effector immune cells express an engineered MHC class I or an MHC class II complex as defined herein.

The allotransplantation may comprise adoptive transfer of allogeneic or autologous immune cells.

In a thirteenth aspect, there is provided an allotransplant which has been depleted of alloreactive immune cells by a method according to the twelfth aspect of the invention.

### DETAILED DESCRIPTION

Some clinical applications involve generating effector immune cells which recognize and deplete a subset of normal immune cells by recognizing their antigen-recognition receptor.

In this situation the targeted, normal immune cell can “fight back”, causing depletion of the effector immune cell. The present invention is concerned with engineering the effector immune cell so that it has an immunological “advantage” over the target immune cell, so that when a synapse is formed between the effector immune cell and the targeted immune cell, the effector immune cell will prevail.

There are various situations in which this effector cell “fight-back” can occur, including:

(i) where the effector immune cell expresses a CAR which specifically binds the T-cell receptor of a T cell;
(ii) where the effector immune cell expresses an engineered MHC I or II complex so that it depletes alloreactive or autoreactive T cells.

These situations are explained in more detail below.

### Chimeric Antigen Receptors Against TCR Complex

The effector immune cell of the present invention may express a chimeric antigen receptor (CAR). In particular, it may express a CAR which specifically binds a component of the T-cell receptor (TCR) or TCR:CD3 complex.

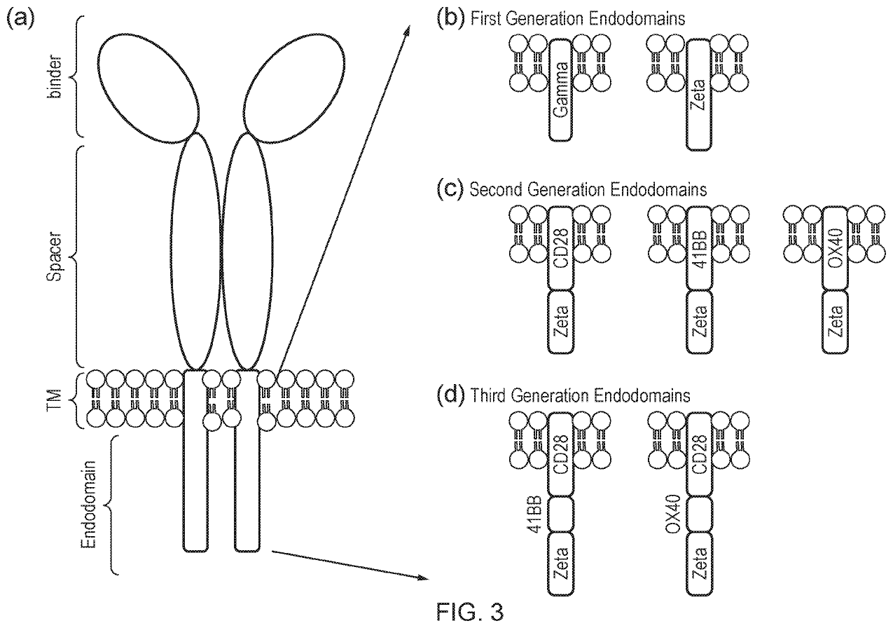

A classical chimeric antigen receptor (CAR) is a chimeric type I trans-membrane protein which connects an extracellular antigen-recognizing domain (binder) to an intracellular signalling domain (endodomain) (see FIG. 3). The binder is typically a single-chain variable fragment (scFv) derived from a monoclonal antibody (mAb), but it can be based on other formats which comprise an antibody-like antigen binding site. A spacer domain may be used to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41 BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general structure:

Antigen binding domain-spacer domain-transmembrane domain-intracellular signaling domain (endodomain).

### Antigen Binding Domain

The antigen binding domain is the portion of the CAR which recognizes antigen. In a classical CAR, the antigen-binding domain comprises: a single-chain variable fragment (scFv) derived from a monoclonal. CARs have also been produced with domain antibody (dAb), VHH or Fab-based antigen binding domains.

Alternatively a CAR may comprise a ligand for the target antigen. For example, B-cell maturation antigen (BCMA)-binding CARs have been described which have an antigen binding domain based on the ligand a proliferation inducing ligand (APRIL).

### Spacer

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

A variety of sequences are commonly used as spacers for CAR, for example, an IgG1 Fc region, an IgG1 hinge, or a human CD8 stalk.

WO2016/151315 describes spacers which form coiled-coil domains and form multimeric CARs. For example, it describes a spacer based on the cartilage-oligomeric matrix protein (COMP) which forms pentamers. A COMP spacer may comprise the sequence shown as SEQ ID No. 1 or a truncated version thereof which retains the capacity to form coiled-coils and therefore multimers.

(COMP spacer)
SEQ ID No. 1
DLGPQMLRELQETNAALQDVRELLRQQVREITFLKNTVMECDACG

### Transmembrane Domain

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

### Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:

(i) an ITAM-containing endodomain, such as the endodomain from CD3 zeta; and/or
(ii) a co-stimulatory domain, such as the endodomain from CD28 or ICOS; and/or
(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The CAR of the present invention may therefore comprise an antigen-binding component comprising an antigen-binding domain and a transmembrane domain; which is capable of interacting with a separate intracellular signalling component comprising a signalling domain.

The vector of the invention may express a CAR signalling system comprising such an antigen-binding component and intracellular signalling component.

The CAR may comprise a signal peptide so that when it is expressed inside a cell, the nascent protein is directed to the endoplasmic reticulum and subsequently to the cell surface, where it is expressed. The signal peptide may be at the amino terminus of the molecule.

### Target Antigen

A ‘target antigen’ is an entity which is specifically recognised and bound by the antigen-binding domain of a CAR.

The target antigen may be an antigen present on a cancer cell, for example a tumour-associated antigen.

Various tumour associated antigens (TAA) are known, as shown in the following Table 1. The CAR may be capable of binding such a TAA.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The effector immune cell of the invention may bind to the T-cell receptor (TCR) complex on a target T-cell. In particular, the effector immune cell of the invention may bind to the TCR β-constant region (TRBC) of a TCR complex on a target T-cell.

The T-cell receptor (TCR) is expressed on the surface of T lymphocytes and is responsible for recognizing antigens bound to major histocompatibility complex (MHC) molecules. When the TCR engages with antigenic peptide and MHC (peptide/MHC), the T lymphocyte is activated through a series of biochemical events mediated by associated enzymes, co-receptors, specialized adaptor molecules, and activated or released transcription factors.

The TCR is a disulfide-linked membrane-anchored heterodimer normally consisting of the highly variable alpha (a) and beta (p) chains expressed as part of a complex with the invariant CD3 chain molecules. T-cells expressing this receptor are referred to as α:β (or αβ) T-cells (˜95% total T-cells). A minority of T-cells express an alternate receptor, formed by variable gamma (γ) and delta (δ) chains, and are referred to as γδ T-cells (˜5% total T cells).

Each α and β chain is composed of two extracellular domains: Variable (V) region and a Constant (C) region, both of Immunoglobulin superfamily (IgSF) domain forming antiparallel β-sheets. The constant region is proximal to the cell membrane, followed by a transmembrane region and a short cytoplasmic tail, while the variable region binds to the peptide/MHC complex. The constant region of the TCR consists of short connecting sequences in which a cysteine residue forms disulfide bonds, which forms a link between the two chains.

The variable domains of both the TCR α-chain and β-chain have three hypervariable or complementarity determining regions (CDRs). The variable region of the β-chain also has an additional area of hypervariability (HV4), however, this does not normally contact antigen and is therefore not considered a CDR.

The TCR also comprises up to five invariant chains γ,δ,ε (collectively termed CD3) and ζ. The CD3 and ζ subunits mediate TCR signalling through specific cytoplasmic domains which interact with second-messenger and adapter molecules following the recognition of the antigen by αβ or γδ. Cell-surface expression of the TCR complex is preceded by the pair-wise assembly of subunits in which both the transmembrane and extracellular domains of TCR α and β and CD3 γ and δ play a role.

TCRs are therefore commonly composed of the CD3 complex and the TCR α and β chains, which are in turn composed of variable and constant regions.

The locus (Chr7:q34) which supplies the TCR β-constant region (TRBC) has duplicated in evolutionary history to produce two almost identical and functionally equivalent genes: TRBC1 and TRBC2, which differ by 4 amino acids in the mature protein. Each TCR will comprise, in a mutually exclusive fashion, either TRBC1 or TRBC2 and as such, each αβ T-cell will express either TRBC1 or TRBC2, in a mutually exclusive manner.

The effector immune cell may be capable of selectively binding to either TRBC1 or TRBC2 in a mutually exclusive manner.

As explained above, each αβ T-cell expresses a TCR which comprises either TRBC1 or TRBC2. In a clonal T-cell disorder, such as a T-cell lymphoma or leukaemia, malignant T-cells derived from the same clone will all express either TRBC1 or TRBC2.

When a TRBC1- or TRBC2-specific CAR-T cell is administered to patient having a T-cell lymphoma or leukaemia, the result is selective depletion of the malignant T-cells, together with normal T-cells which express the same TRBC as the malignant T-cells, but such treatment does not cause significant depletion of normal T-cells expressing the other TRBC from the malignant T-cells.

Because the TRBC selective CAR-T cell does not cause significant depletion of normal T-cells expressing the other TRBC from the malignant T-cells it does not cause depletion of the entire T-cell compartment. Retention of a proportion of the subject's T-cell compartment (i.e. T-cells which do not express the same TRBC as the malignant T-cell) results in reduced toxicity and reduced cellular and humoral immunodeficiency, thereby reducing the risk of infection.

### TRBC1 Binding CAR-T Cells

CAR-T cells specific for TRBC1 and TRBC2 are described in International application No. WO2015/132598.

A CAR which selectively binds TRBC1 may have a variable heavy chain (VH) and a variable light chain (VL) which comprises the following complementarity determining regions (CDRs):

VH CDR1:
(SEQ ID No. 2)
GYTFTGY;
VH CDR2:
(SEQ ID No. 3)
NPYNDD;
VH CDR3:
(SEQ ID No. 4)
GAGYNFDGAYRFFDF;
VL CDR1:
(SEQ ID No. 5)
RSSQRLVHSNGNTYLH;
VL CDR2:
(SEQ ID No. 6)
RVSNRFP;
and
VL CDR3:
(SEQ ID No. 7)
SQSTHVPYT.

The one or more CDRs each independently may or may not comprise one or more amino acid mutations (eg substitutions) compared to the sequences given as SEQ ID No. 8 to 13, provided that the resultant antibody retains the ability to selectively bind to TRBC1.

The antigen-binding domain of a TRBC1 selective CAR may comprise a variable heavy chain (VH) having the amino acid sequence shown as SEQ ID No. 8 and a variable light chain (VL) having the amino acid sequence shown as SEQ ID No. 9.

(hJovi-1 VH)
SEQ ID No. 8
QVQLVQSGAEVKKPGASVKVSCKASGYTFTGYVMHWVRQAPGQGLEWMG
FINPYNDDIQSNERFRGRVTMTRDTSISTAYMELSRLRSDDTAVYYCAR
GAGYNFDGAYRFFDFWGQGTMVTVSS
(hJovi-1 VL)
SEQ ID No. 9
DIVMTQSPLSLPVTPGEPASISCRSSQRLVHSNGNTYLHWYLQKPGQSP
RLLIYRVSNRFPGVPDRFSGSGSGTDFTLKISRVEAEDVGVYYCSQSTH
VPYTFGQGTKLEIK

The CAR may comprise an ScFv having the amino acid sequence shown as SEQ ID No. 10.

Jovi-1 scFv
SEQ_ID_10
EVRLQQSGPDLIKPGASVKMSCKASGYTFTGYVMHWWKQRPGQGLEWIG
FINPYNDDIQSNERFRGKATLTSDKSSTTAYMELSSLTSEDSAVYYCAR
GAGYNFDGAYRFFDFWGQGTTLTVSSGGGGSGGGGSGGGGSDVVMTQSP
LSLPVSLGDQASISCRSSQRLVHSNGNTYLHWYLQKPGQSPKLLIYRVS
NRFPGVPDRFSGSGSGTDFTLKISRVEAEDLGIYFCSQSTHVPYTFGGG
TKLEIKR

### TRBC1 Binding CAR-T Cells

CAR-T cells specific for TRBC2 are described in International application No. PCT/GB2019/053100.

A TRBC 2-specific CAR may have an antigen-binding domain which comprises at least one mutation in the VH domain compared to a reference antibody having a VH domain with the sequence shown in SEQ ID NO: 7 and a VL domain with the sequence shown in SEQ ID NO: 8, in which at least one mutation in the VH domain is selected from T28K, Y32K and A100N. Such an antigen-binding domain should display an increased affinity for TRBC2 over the TRBC-1 binding reference antibody, JOVI-1.

The variant antigen-binding domain may comprise at least two mutations in the VH domain selected from T28K, Y32K and A100N. For example, it may comprise mutations Y32K and A100N. The variant antigen-binding domain may further comprise mutation T28R in the VH domain or, alternatively, mutation G31K in the VH domain.

The variant antigen-binding domain may comprise T28K, Y32K and A100N mutations.

The variant antigen-binding domain may further comprise at least one mutation at a position selected from the group consisting of V2, Y27, G31, R98, Y102, N103, and A107 in the VH domain, N35 in the VL domain, and R55 in the VL domain. The at least one further mutation may be selected from:

a) in the VH domain:
V2K, V2R,
Y27F, Y27M, Y27N, Y27W,
G31K, G31R, G31S,
R98K,
Y102F, Y102L,
N103A, N103E, N103F, N103H, N103L, N103M, N103Q, N103S, N103W, N103Y,
A107S,
   and
   b) in the VL domain:
N35M, N35F, N35Y, N35K, N35R, and
R55K.

The variant antigen-binding domain may be selected from a variant antigen-binding domain comprising the following mutation combinations:
T28K, Y32F, A100N in the VH domain and N35K in the VL domain,
T28K, Y32F, A100N in the VH domain,
T28K, Y32F, A100N, Y27N in the VH domain
T28K, Y32F, A100N, G31K in the VH domain
T28K, Y32F, A100N, Y27M in the VH domain
T28K, Y32F, A100N, Y27W in the VH domain
T28K, Y32F, A100N in the VH domain and R55K in the VL domain,
T28K, Y32F, A100N, N103H in the VH domain
T28K, Y32F, A100N, N103A in the VH domain
T28K, Y32F, A100N, N103Y in the VH domain
T28K, Y32F, A100N in the VH domain and N35R in the VL domain,
T28K, Y32F, A100N, N103S in the VH domain and N35M in the VL domain,
T28K, Y32F, A100N, N103M, in the VH domain,
T28K, Y32F, A100N, N103W in the VH domain and N35R in the VL domain,
T28K, Y32F, A100N in the VH domain and N35F in the VL domain,
T28K, Y32F, A100N, N103S in the VH domain and N35K in the VL domain,
T28K, Y32F, A100N, R98K in the VH domain,
T28K, Y32F, A100N, N103S in the VH domain and N35R in the VL domain,
T28K, Y32F, A100N, N103L in the VH domain,
T28K, Y32F, A100N, N103S in the VH domain and N35F in the VL domain,
T28K, Y32F, A100N, N103S in the VH domain and N35Y in the VL domain,
T28K, Y32F, A100N, N103L in the VH domain and N35M in the VL domain,
T28K, Y32F, A100N, N103L in the VH domain and N35R in the VL domain,
T28K, Y32F, A100N, N103W in the VH domain and N35K in the VL domain,
T28K, Y32F, A100N, N103L in the VH domain and N35Y in the VL domain,
T28K, Y32F, A100N, N103F in the VH domain,
T28K, Y32F, A100N, N103W in the VH domain,
T28K, Y32F, A100N, N103L in the VH domain and N35K in the VL domain,
T28K, Y32F, A100N, N103L in the VH domain and N35F in the VL domain,
T28K, Y32F, A100N, N103W in the VH domain and N35M in the VL domain,
T28K, Y32F, A100N, N103F in the VH domain and N35Y in the VL domain,
T28K, Y32F, A100N, Y27F in the VH domain,
T28K, Y32F, A100N, N103Q in the VH domain,
T28K, Y32F, A100N, N103S in the VH domain,
T28K, Y32F, A100N, N103M in the VH domain and N35F in the VL domain,
T28K, Y32F, A100N, N103F in the VH domain and N35M in the VL domain,
T28K, Y32F, A100N, N103F in the VH domain and N35F in the VL domain,
T28K, Y32F, A100N, G31R in the VH domain,
T28K, Y32F, A100N, N103W in the VH domain and N35F in the VL domain,
T28K, Y32F, A100N, V2R in the VH domain,
T28K, Y32F, A100N, G31S in the VH domain,
T28K, Y32F, A100N, A107S in the VH domain,
T28K, Y32F, A100N, N103E in the VH domain and N35M in the VL domain,
T28K, Y32F, A100N, V2K in the VH domain,
T28K, Y32F, A100N, N103E in the VH domain
T28K, Y32F, A100N, Y102F, N103M in the VH domain and N35K in the VL domain,
T28K, Y32F, A100N, Y102F, N103M in the VH domain and N35F in the VL domain,
T28K, Y32F, A100N, Y102F, N103M in the VH domain and N35R in the VL domain,
T28K, Y32F, A100N, Y102F in the VH domain and N35R in the VL domain,
T28K, Y32F, A100N, N103M in the VH domain and N35M in the VL domain,
T28K, Y32F, A100N, N103M in the VH domain and N35Y in the VL domain,
T28K, Y32F, A100N, N103M in the VH domain and N35R in the VL domain,
T28K, Y32F, A100N, N103F in the VH domain and N35K in the VL domain,
T28K, Y32F, A100N, Y102L, N103W in the VH domain and N35R in the VL domain,
T28K, Y32F, A100N, Y102L, N103W in the VH domain and N35K in the VL domain,
T28K, Y32F, A100N, Y102F in the VH domain, and
T28K, Y32F, A100N, Y102L, N103M in the VH domain and N35R in the VL domain.

The variant antigen-binding domain may comprise T28K, Y32F, A100N mutations in the VH domain and N35K mutation in the VL domain,

The variant antigen-binding domain may comprise T28K, Y32F, and A100N mutations in the VH domain.

### Engineered MHC I or II Complexes

The major histocompatibility complex (MHC) is a large locus on vertebrate's DNA containing a set of closely linked polymorphic genes that code for cell surface proteins essential for the acquired immune system. MHC is the tissue-antigen that allows the immune system (more specifically T cells) to bind to, recognize, and tolerate itself (autorecognition). MHC is also the chaperone for intracellular peptides that are complexed with MHCs and presented to T cell receptors (TCRs) as potential foreign antigens. MHC interacts with TCR and its co-receptors to optimize binding conditions for the TCR-antigen interaction, in terms of antigen binding affinity and specificity, and signal transduction effectiveness.

Essentially, the MHC-peptide complex is a complex of auto-antigen/allo-antigen. Upon binding, T cells should in principle tolerate the auto-antigen, but activate when exposed to the allo-antigen.

MHC molecules bind to both T cell receptor and CD4/CD8 co-receptors on T lymphocytes, and the antigen epitope held in the peptide-binding groove of the MHC molecule interacts with the variable Ig-Like domain of the TCR to trigger T-cell activation.

MHC class I molecules are expressed in all nucleated cells and also in platelets—in essence all cells but red blood cells. MHC class I presents peptide epitopes to cytotoxic T lymphocytes (CTLs). A CTL expresses CD8 receptors, in addition to TCRs. When a CTL's CD8 receptor docks to a MHC class I molecule, if the CTL's TCR fits the epitope within the MHC class I molecule, the CTL triggers the cell to undergo programmed cell death by apoptosis. Thus, MHC class I helps mediate cellular immunity, a primary means to address intracellular pathogens, such as viruses and some bacteria. In humans, MHC class I comprises HLA-A, HLA-B, and HLA-C molecules.

MHC-I molecules are heterodimers, they have a polymorphic heavy α-subunit whose gene occurs inside the MHC locus and small invariant β2 microglobulin subunit whose gene is located usually outside of it. The polymorphic heavy chain of MHC-I molecule contains N-terminal extra-cellular region composed by three domains, α1, α2, and α3, a transmembrane helix to hold MHC-I molecule on the cell surface and a short cytoplasmic tail. Two domains, α1 and α2 form a deep peptide-binding groove between two long α-helices and the floor of the groove is formed by eight p-strands. Immunoglobulin-like domain α3 is involved in the interaction with CD8 co-receptor. β2 microglobulin provides stability of the complex and participates in the recognition of peptide-MHC class I complex by the CD8 co-receptor. The peptide is non-covalently bound to MHC-1, it is held by the several pockets on the floor of the peptide-binding groove. Amino acid side-chains that are most polymorphic in human alleles fill up the central and widest portion of the binding groove, while conserved side-chains are clustered at the narrower ends of the groove.

MHC class II can be conditionally expressed by all cell types, but normally occurs only on “professional” antigen-presenting cells (APCs): macrophages, B cells, and especially dendritic cells (DCs). An APC takes up an antigenic protein, performs antigen processing, and returns a molecular fraction of the protein—an antigenic epitope—and displays it on the APC's surface coupled within an MHC class II molecule (antigen presentation). On the cell's surface, the epitope can be recognized by immunologic structures like T cell receptors (TCRs).

On surface of helper T cells are CD4 receptors, as well as TCRs. When a naive helper T cell's CD4 molecule docks to an APC's MHC class II molecule, its TCR can meet and bind the epitope coupled within the MHC class II. This event primes the naive T cell.

Class II MHC molecules are also heterodimers, genes for both α and β subunits are polymorphic and located within MHC class II subregion. The peptide-binding groove of MHC-II molecules is forms by N-terminal domains of both subunits of the heterodimer, α1 and β1; unlike MHC-1 molecules, where two domains of the same chain are involved. In addition, both subunits of MHC-II contain transmembrane helix and immunoglobulin domains α2 or β2 that can be recognized by CD4 co-receptors. In this way MHC molecules chaperone which type of lymphocytes may bind to the given antigen with high affinity, since different lymphocytes express different T-Cell Receptor (TCR) co-receptors.

The effector immune cell of the present invention may comprise an MHC class I polypeptide; an MHC class II polypeptide; or β-2 microglobulin, linked to an intracellular signalling domain.

### Peptide-Specific Approaches

CD8+ T cells are key mediators of transplant rejection and graft-versus host disease and contribute to the pathogenesis of autoimmune diseases. As explained above, it is to convert TCR ligands into T-cell activation receptors by expressing a β2 microglobulin polypeptide which comprises an intracellular signalling domain attached to one end and an antigenic peptide attached to the other end via a linker. Cells engineered to express such a molecule were found to express a high level of surface peptide-class I complexes, presenting the antigenic peptide and to respond to antibodies and target T-cells in a peptide specific manner. By expressing such a peptide-linker-signalling domain polypeptide in effector immune cells such as T-cells, it is possible to specifically target pathogenic CD8-T cells recognising a particular antigenic peptide.

Thus, the effector immune cells of the present invention may comprise an engineered MHC class I complex which comprises a molecule having the following structure:

peptide-L-B2M-endo
in which:
“peptide” is a peptide which binds the peptide binding groove of the MHC class I α-chain;
“L” is a linker
“B2M” is β-2 microglobulin; and
“endo” is an intracellular signalling domain.

The peptide may be an alloantigen or an autoantigen.

Autoimmune disorders are characterized by reactivity of the immune system to an endogenous antigen, with consequent injury to tissues. More than 80 chronic autoimmune diseases have been characterized that affect virtually almost every organ system in the body. The most common autoimmune diseases are insulin dependent diabetes mellitus (IDDM), multiple sclerosis (MS), systemic lupus erythematosus (SLE), rheumatoid arthritis, several forms of anemia (pernicious, plastic, hemolytic), thyroiditis, and uveitis.

Allograft rejection typically results from an overwhelming adaptive immune response against foreign organ or tissue. It is the major risk factor in organ transplantation and is the cause of post-transplantation complications. A major complication associated with bone marrow (BM) transplantation, known as graft versus-host (GVH) reaction or graft-versus-host disease (GVHD), occurs in at least half of patients when grafted donor lymphocytes, injected into an allogeneic recipient whose immune system is compromised, begin to attack the host tissue, and the host's compromised state prevents an immune response against the graft.

The linker connects the peptide to β-2 microglobulin and provides flexibility such that the peptide can bind the peptide-binding groove of an associated MHC molecule. It may, for example, comprise between 5-20 amino acids, or 10-15 amino acids.

The molecule may also comprise a peptide bridge to bridge β-2 microglobulin to the cell membrane. The peptide bridge may comprise the 13 membrane proximal amino acids of the extracellular portion of HLA-A2 which has the sequence

(SEQ ID No. 11)
LRWEPSSNPTIPI

The molecule may comprise a membrane-targeting domain, such as a transmembrane domain. By way of example, the transmembrane domains of CD8alpha and CD28 are shown as SEQ ID NO: 12 and SEQ ID NO: 13, respectively.

(CD8 alpha transmembrane domain)
SEQ ID NO: 12
IYIWAPLAGTCGVLLLSLVITLY
(CD28 transmembrane domain)
SEQ ID NO: 13
FWWLVVVGGVLACYSLLVTVAFIFVWR

The amino acid sequence of human β-2 microglobulin is available from Uniprot Accession No. P61769 and is shown below as SEQ ID No. 14.

(human β-2 microglobulin)
SEQ ID No. 14
MSRSVALAVLALLSLSGLEAIQRTPKIQVYSRHPAENGKSNFLNCYVSG
FHPSDIEVDLLKNGERIEKVEHSDLSFSKDWSFYLLYYTEFTPTEKDEY
ACRVNHVTLSQPKIVKWDRDM

The engineered MHC class I complex may comprise a variant of the β-2 microglobulin sequence shown as SEQ ID No. 14, for example a variant having at least 80%, 90%, 95% or 99% amino acid identity to the sequence shown as SEQ ID 14, provided that the resultant peptide-L-B2M-endo molecule retains the capacity to associate with MHC class I α chain.

The endodomain from human CD3zeta has the sequence shown as SEQ ID No. 15.

(human CD3ζ endodomain)
SEQ ID No. 15
SRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTY
DALHMQALPPR

The engineered MHC class I complex may comprise an intracellular signalling domain having the sequence shown as SEQ ID No. 15 or a variant having at least 80%, 90%, 95% or 99% amino acid identity to the sequence shown as SEQ ID 15, provided that the resultant peptide-L-B2M-endo molecule retains the capacity to trigger activation of the effector immune cell upon TCR recognition

Further intracellular signalling domains and co-stimulatory domains are described below.

### Peptide Agnostic Approaches

It is possible to couple the binding of a MHC class I or II on an effector immune cell to a TCR on a target immune cell to induce—directly or indirectly—signalling in the effector cell. In these approached the MHC signalling systems are capable of presenting the same range of peptides as a corresponding endogenous MHC class I and II molecules. As such, any peptide which is naturally presented by MHC class I or II molecule is presented by the engineered MHC complex. This includes peptides derived from any xenogeneic or junctional sequences, for example derivable from a chimeric antigen receptor expressed by the cell, that may be immunogenic. In an allogeneic setting, this may also include minor histocompatibility antigens. Thus such an engineered MHC class complex will interact with any endogenous, reactive T-cells present in the recipient of the engineered cell through recognition of peptide/MHC complexes. The reactive T-cell can thus be depleted by activation of cytotoxic-mediated cell killing by the cell of the present invention. Hence, a cellular immune response against the cell of the present invention can be reduced.

In this respect, the effector immune cell may comprise a polypeptide capable of co-localizing: an MHC class I polypeptide; an MHC class II polypeptide; or β-2 microglobulin with an intracellular signalling domain.

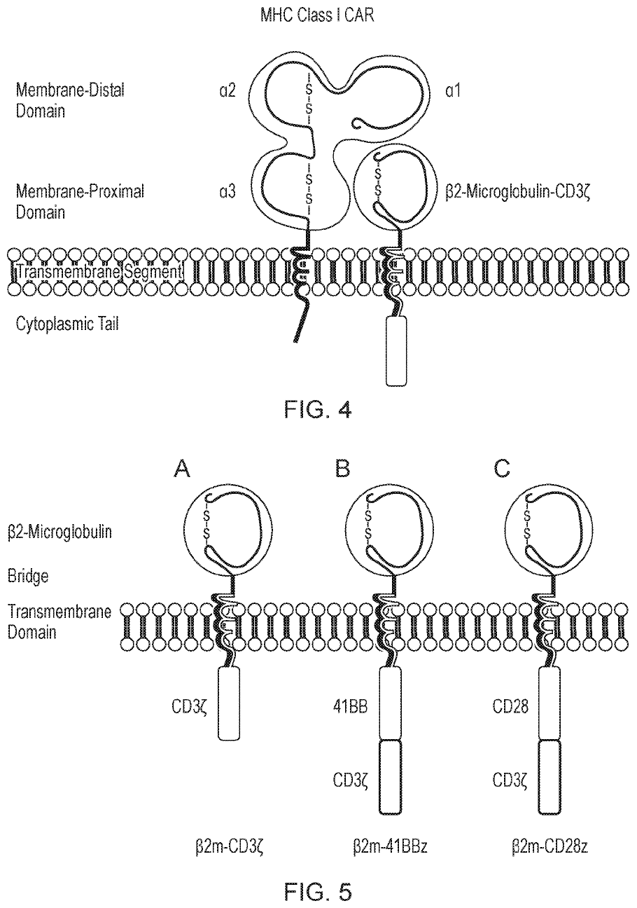

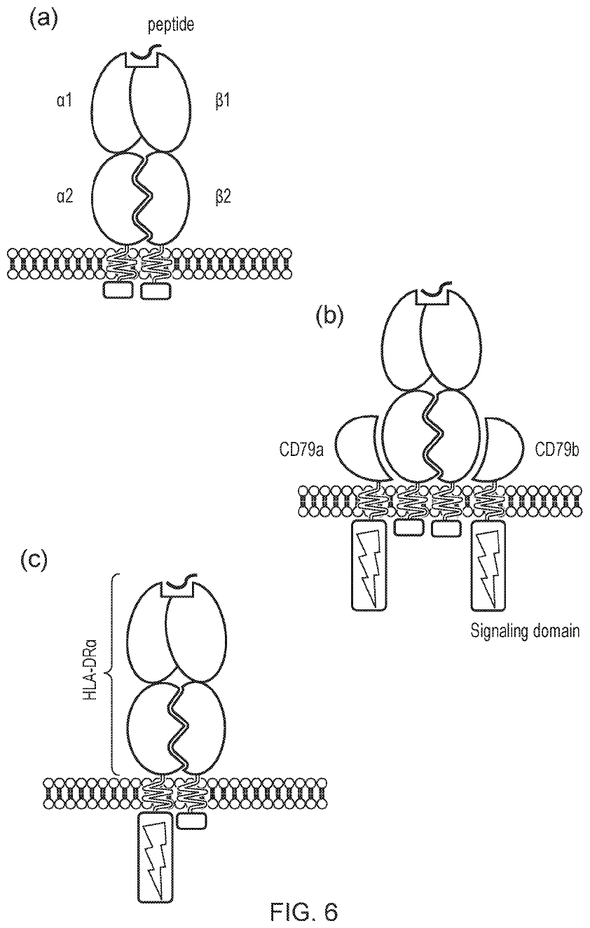

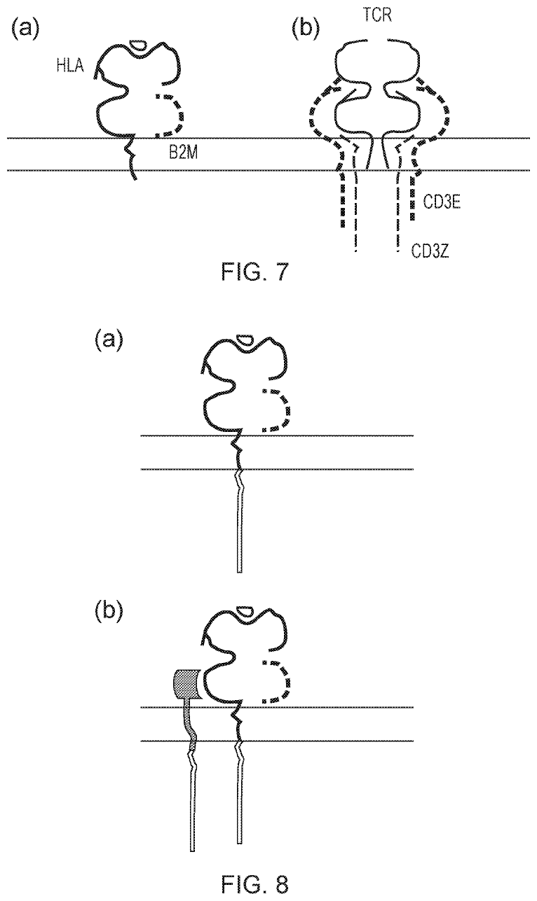

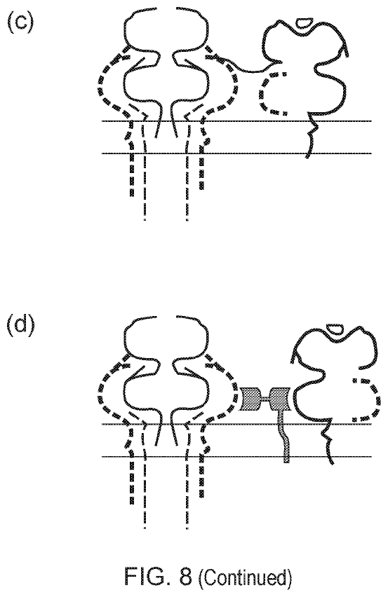

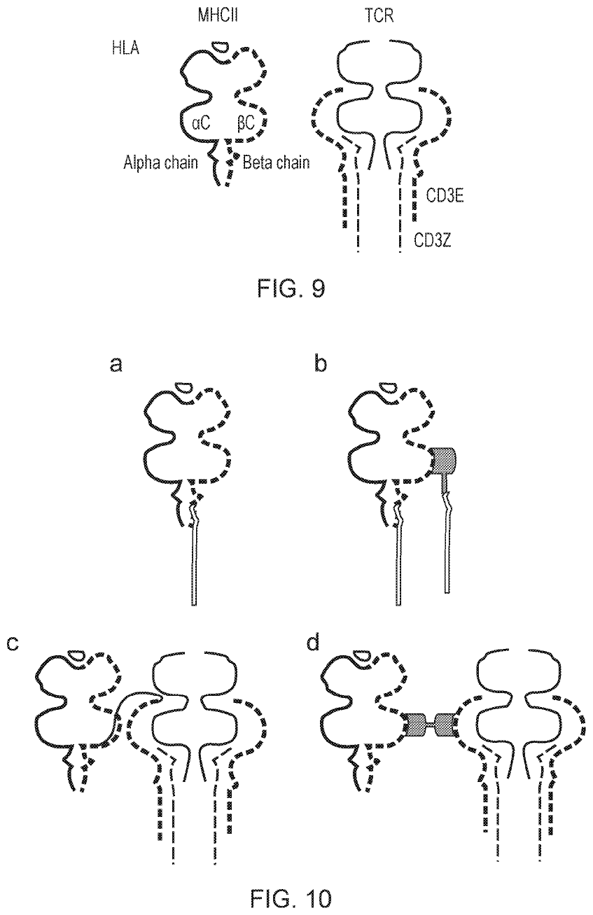

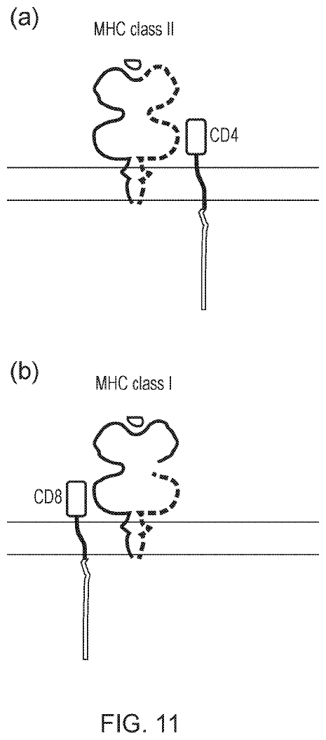

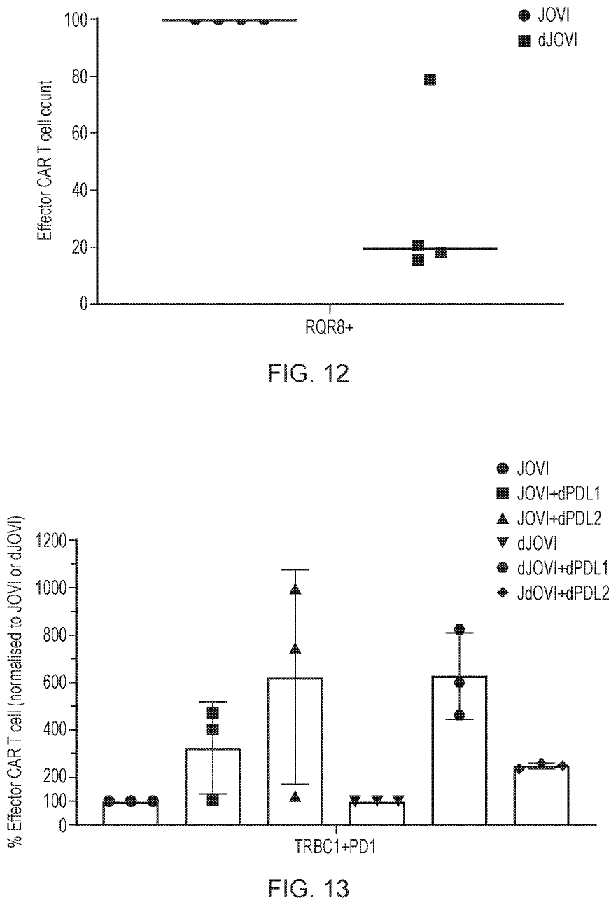

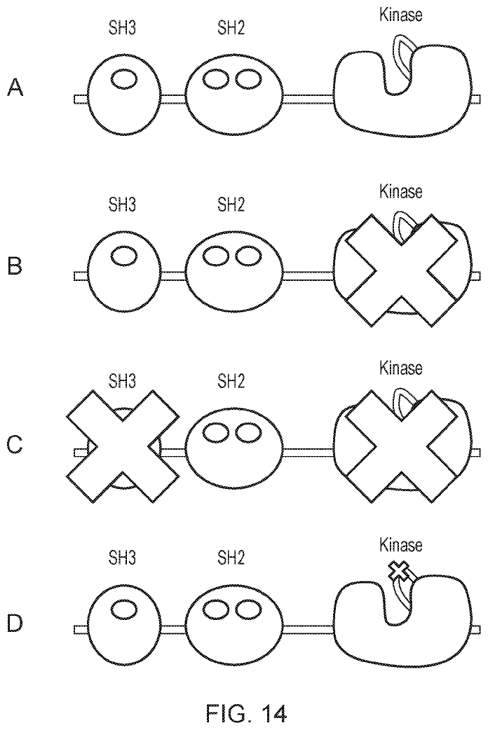

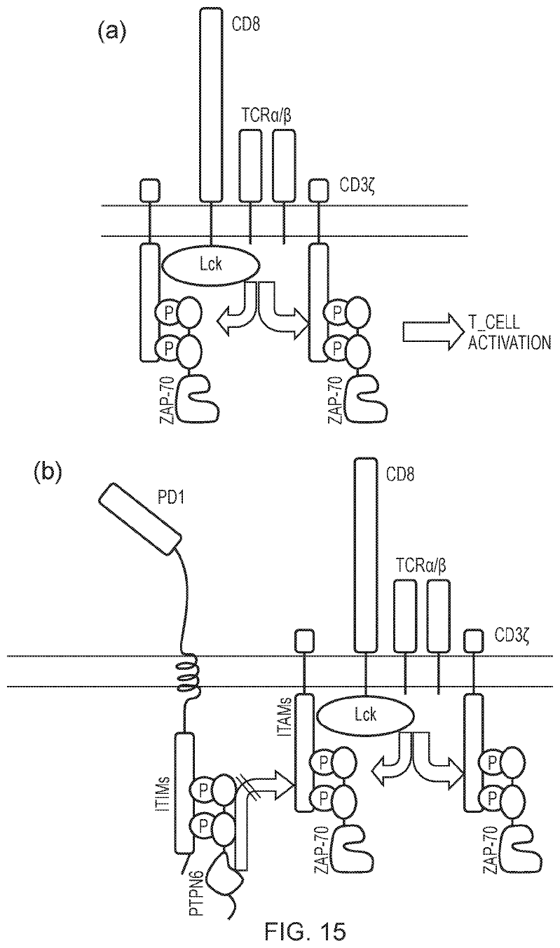

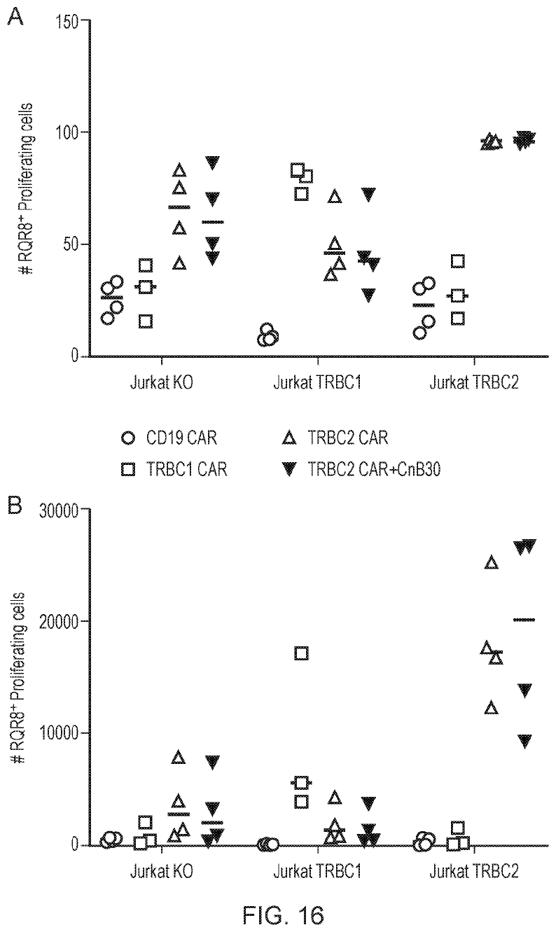

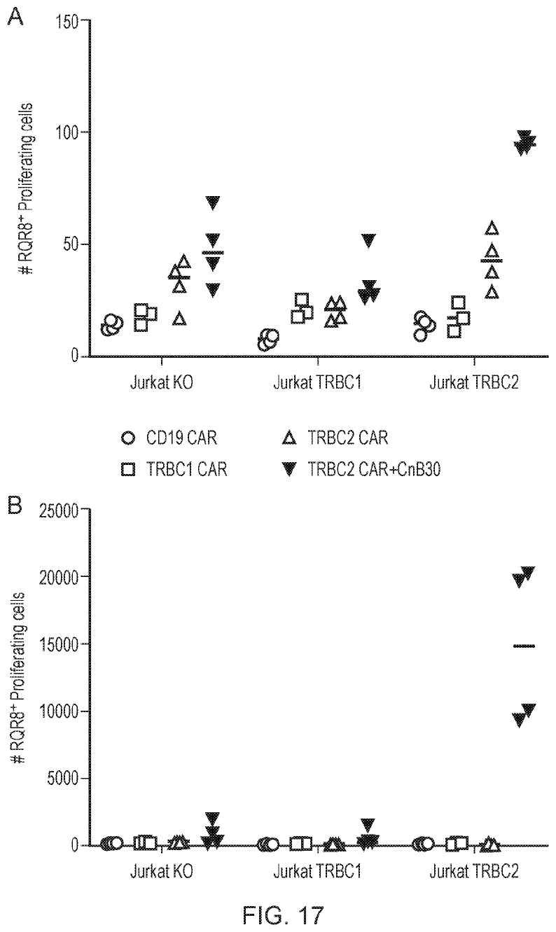

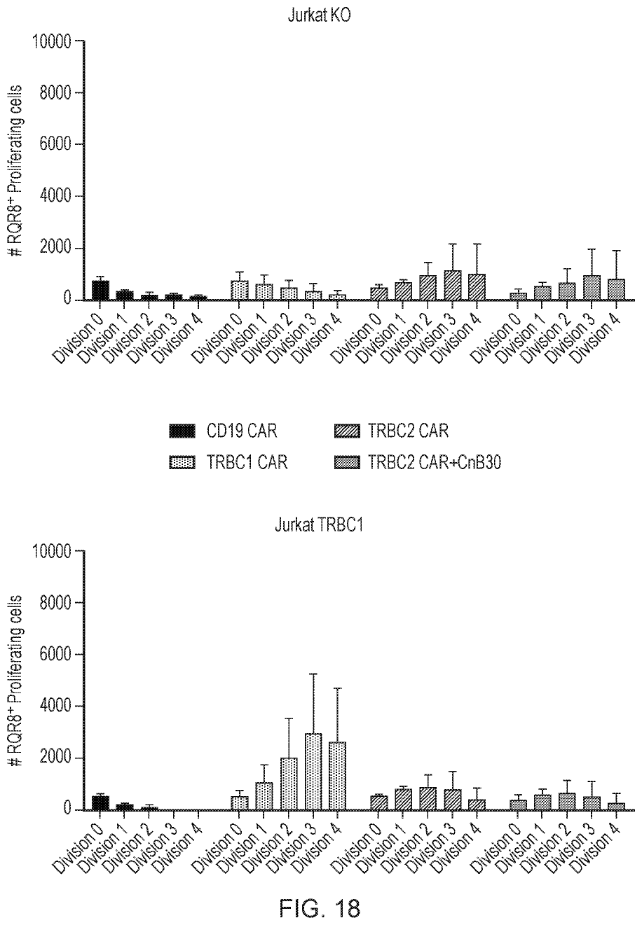

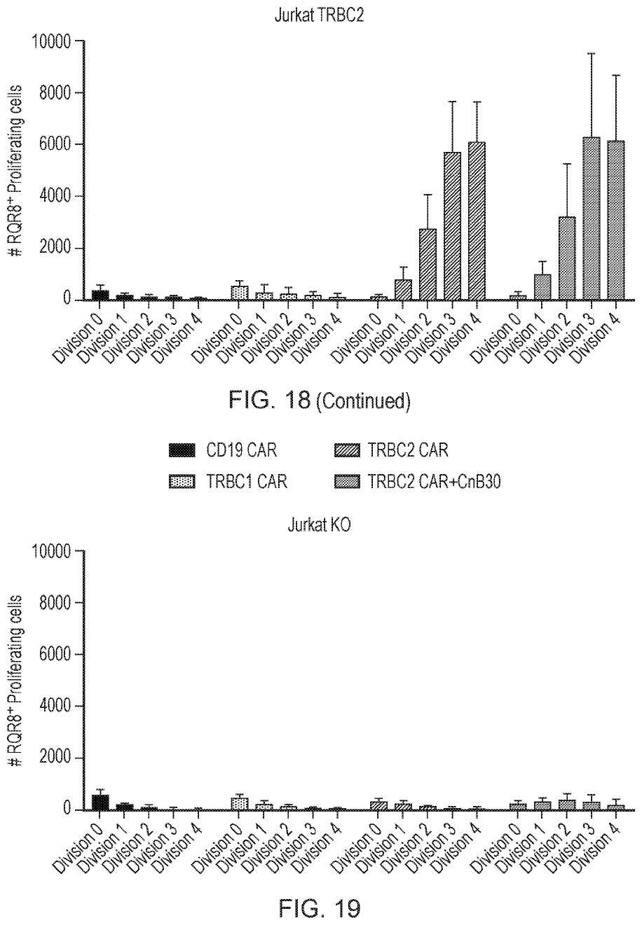

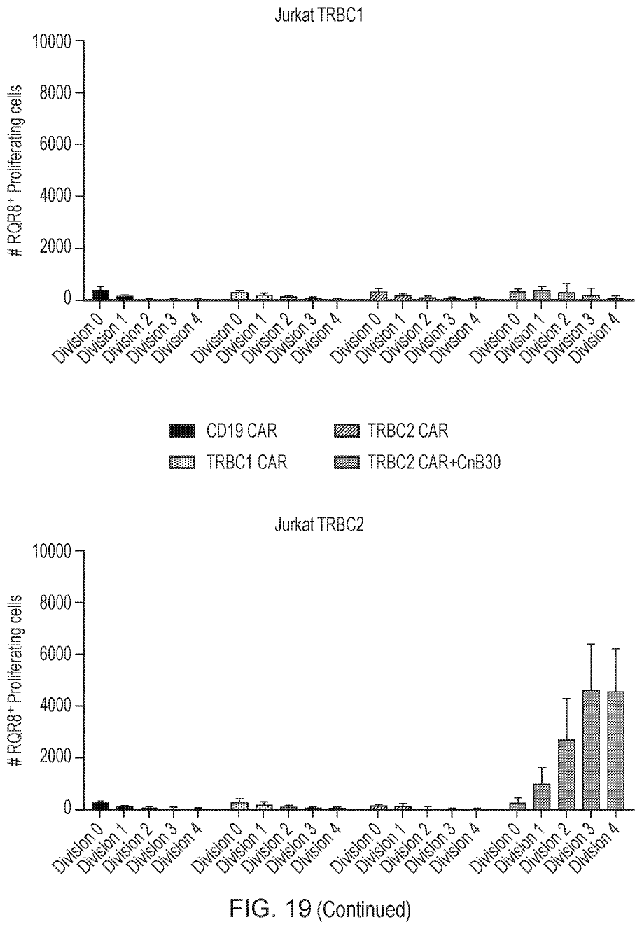

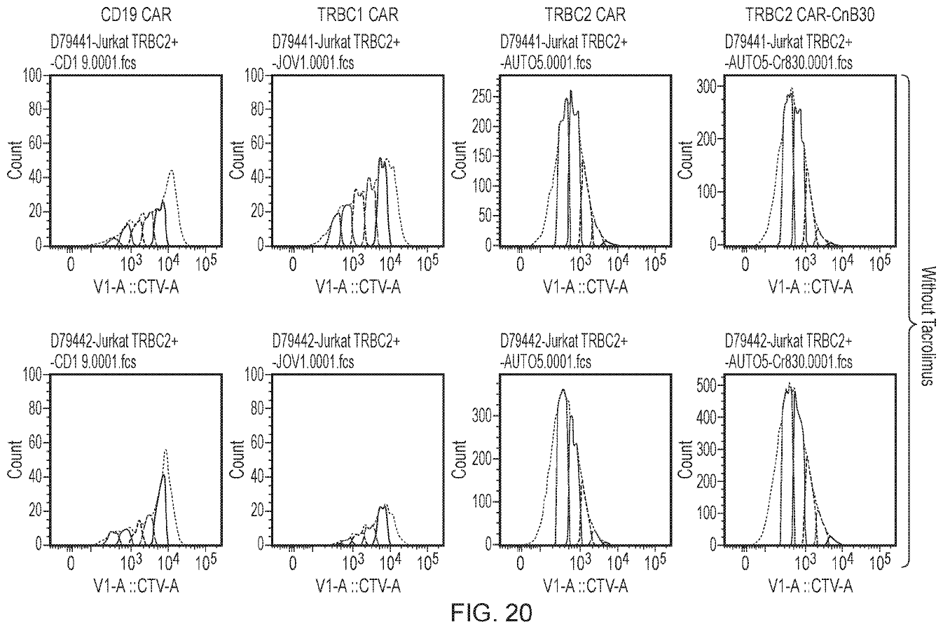

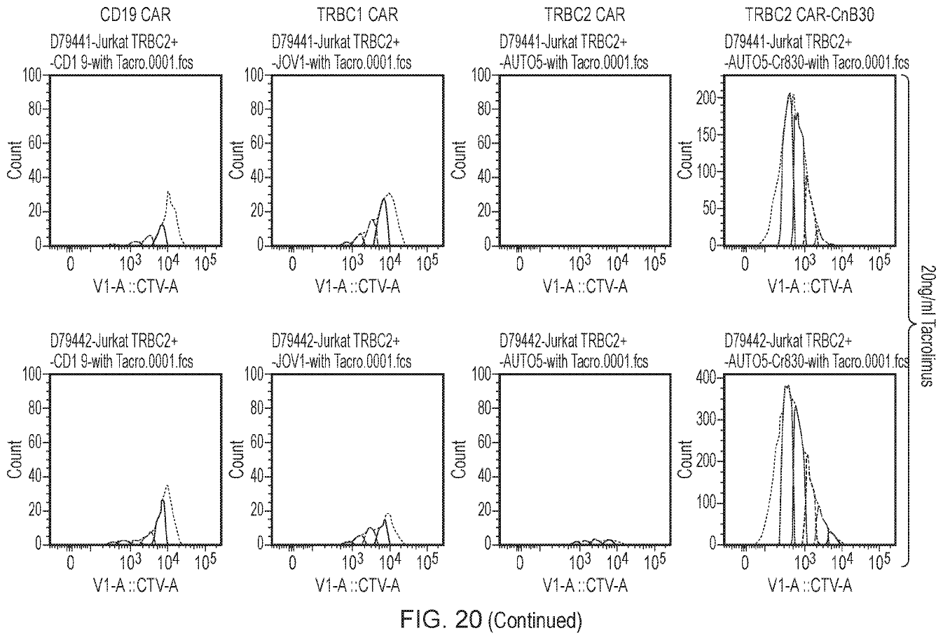

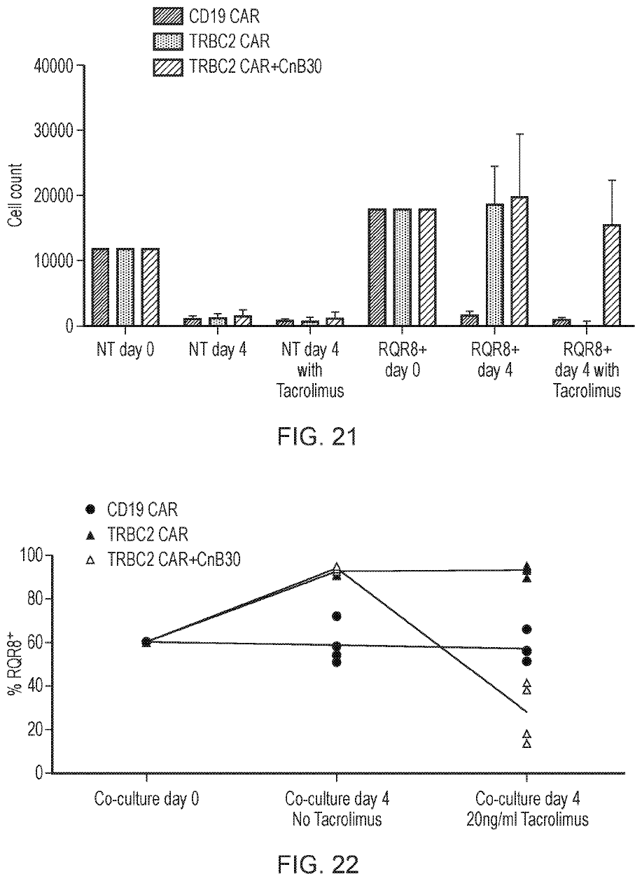

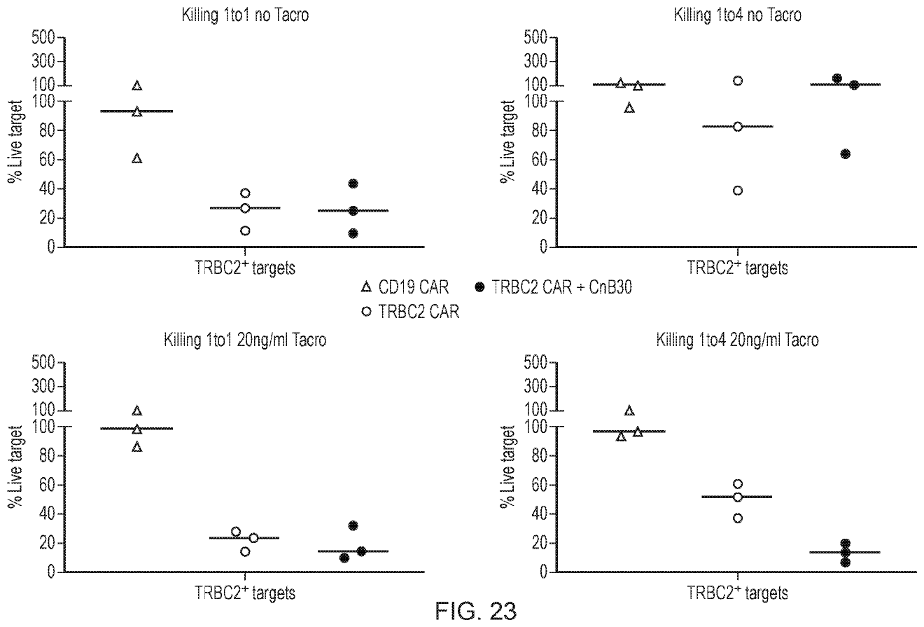

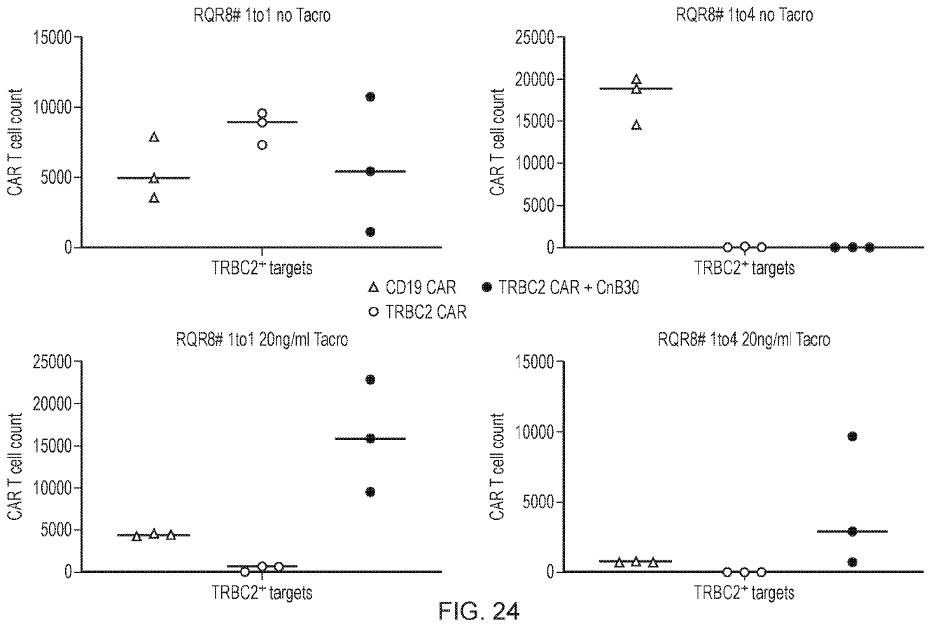

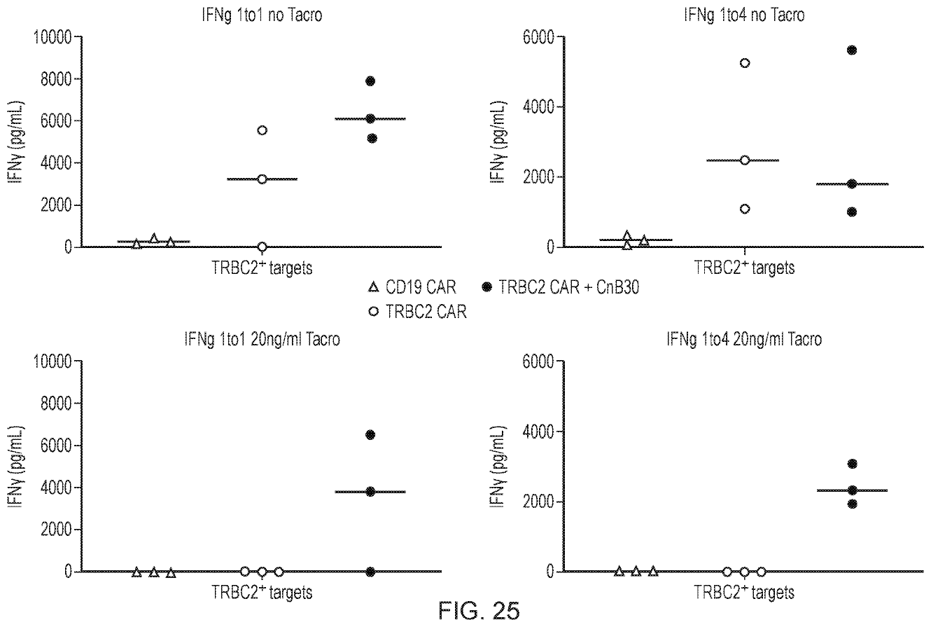

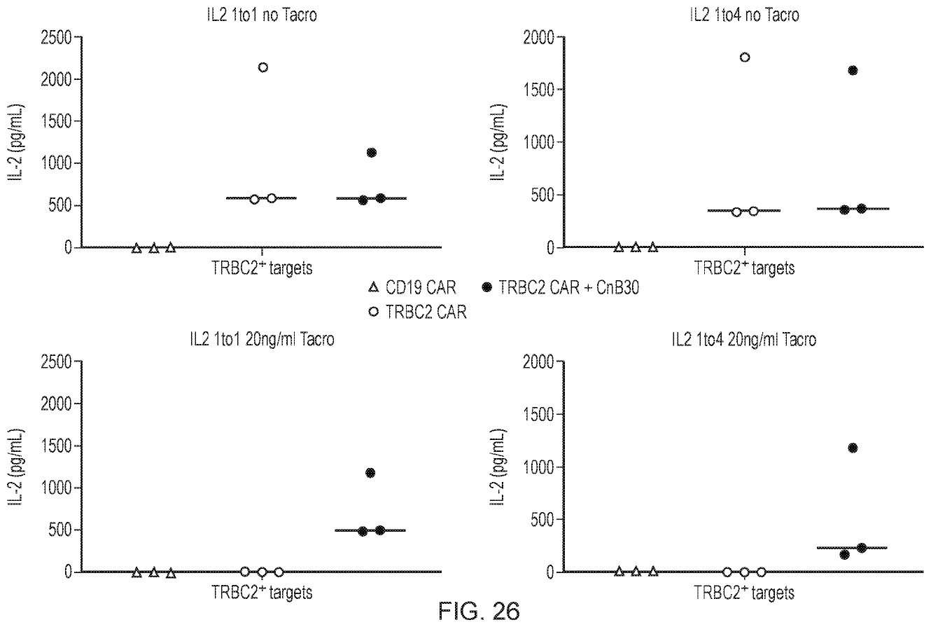

The effector immune cell may comprise:
(i) an engineered polypeptide which comprises the ectodomain from an MHC class I polypeptide or the ectodomain from an MHC class II polypeptide linked to an intracellular signalling domain; or β-2 microglobulin linked to an intracellular signalling domain (see FIGS. 2a, 4, 5, 6c, 8a and 10a);
(ii) an engineered polypeptide which comprises an MHC class I polypeptide or MHC class II polypeptide or β-2 microglobulin linked to linked to a component of the CD3/TCR complex, such as CD3-zeta, CD3-epsilon, CD3-gamma or CD3-delta (see FIGS. 2c, 8c and 10c);
(iii) an engineered polypeptide which comprises a binding domain, such as an antibody-like binding domain, which binds to an MHC class I polypeptide, an MHC class II polypeptide or β-2 microglobulin, linked to an intracellular signalling domain (see FIGS. 8b and 10b);
(iv) an engineered polypeptide which comprises CD79α or CD79β linked to an intracellular signalling domain (see FIG. 6b);
(v) an engineered polypeptide which comprises the MHC class II-binding domain of CD4 linked to an intracellular signalling domain; or the MHC class I-binding domain of CD8 linked to an intracellular signalling domain (see FIG. 11).

Alternatively the effector immune cell may engineered to express a bispecific polypeptide which comprises: (i) a first binding domain which binds to MHC class I polypeptide; an MHC class II polypeptide; β-2 microglobulin; and (ii) a second binding domain which binds to a component of the TCR/CD3 complex (see FIGS. 2b, 8d and 10d).

### HLA Class I

MHC class I molecules are heterodimers that consist of two polypeptide chains, an α polypeptide and β2-microglobulin (b2m). The two chains are linked non-covalently via interaction of b2m and the α3 domain. The α chain is polymorphic and, in humans, encoded by a human leukocyte antigen gene complex (HLA). The b2m subunit is not polymorphic and encoded by the Beta-e macroglobulin gene. HLA gene. HLAs corresponding to MHC class I are HLA-A, HLA-B, HLA-C, HLA-E, HLA-F, HLA-G.

HLA-A, HLA-B and HLA-C are typically very polymorphic whilst HLA-E, HLA-F, HLA-G are less polymorphic.

The engineered polypeptide of the effector cell of the invention may comprise the extracellular domain of HLA-A, HLA-B, HLA-C, HLA-E, HLA-F or HLA-G.

The engineered polypeptide or bispecific polypeptide expressed by the effector cell of the invention may bind HLA-A, HLA-B, HLA-C, HLA-E, HLA-F or HLA-G

The most common haplotypes vary between populations. Accordingly, an effector immune cell according to the present invention may be designed for a certain population with specific common haplotypes. Exemplary class I haplotypes are summarised in the table below:

TABLE 2
Exemplary common haplotypes.

| HLA-A | HLA-B | HLA-C |
|---|---|---|
| HLA-A02 | HLA-B08 | HLA-C01 |
| HLA-A03 | HLA-B07 | |
| HLA-A01 | HLA-B44 | |
| HLA-A29 | HLA-B15 | |
| HLA-A30 | HLA-B35 | |

An amino acid sequence of HLA class I—HLA-A is HLA-A01 as shown as SEQ ID NO: 16:

SEQ ID NO: 16
MAVMAPRTLLLLLSGALALTQTWAGSHSMRYFFTSVSRPGRGEPRFIAV
GYVDDTQFVRFDSDAASQKMEPRAPWIEQEGPEYWDQETRNMKAHSQTD
RANLGTLRGYYNQSEDGSHTIQIMYGCDVGPDGRFLRGYRQDAYDGKDY
IALNEDLRSWTAADMAAQITKRKWEAVHAAEQRRVYLEGRCVDGLRRYL
ENGKETLQRTDPPKTHMTHHPISDHEATLRCWALGFYPAEITLTWQRDG
EDQTQDTELVETRPAGDGTFQKWAAVVVPSGEEQRYTCHVQHEGLPKPL
TLRWELSSQPTIPIVGIIAGLVLLGAVITGAVVAAVMWRRKSSDRKGGS
YTQAASSDSAQGSDVSLTACKV

In the sequence shown as SEQ ID No. 16 to 22:

Ectodomain=unformatted text
Bold/underline=transmembrane
Italics=endodomain

An amino acid sequence of HLA class I—HLA-A is HLA-A02 as shown as SEQ ID NO: 17:

SEQ ID NO: 17
MAVMAPRTLVLLLSGALALTQTWAGSHSMRYFFTSVSRPGRGEPRFIAV
GYVDDTQFVRFDSDAASQRMEPRAPWIEQEGPEYWDGETRKVKAHSQTH
RVDLGTLRGYYNQSEAGSHTVQRMYGCDVGSDWRFLRGYHQYAYDGKDY
IALKEDLRSWTAADMAAQTTKHKWEAAHVAEQLRAYLEGTCVEWLRRYL
ENGKETLQRTDAPKTHMTHHAVSDHEATLRCWALSFYPAEITLTWQRDG
EDQTQDTELVETRPAGDGTFQKWAAVWPSGQEQRYTCHVQHEGLPKPLT
LRWEPSSQPTIPIVGIIAGLVLFGAVITGAVVAAVMWRRKSSDRKGGSY
SQAASSDSAQGSDVSLTACKV

An amino acid sequence of HLA class I—HLA-A is HLA-A-A03 as shown as SEQ ID NO: 18:

SEQ ID NO: 18
MAVMAPRTLLLLLSGALALTQTWAGSHSMRYFFTSVSRPGRGEPRFIAV
GYVDDTQFVRFDSDAASQRMEPRAPWIEQEGPEYWDQETRNVKAQSQTD
RVDLGTLRGYYNQSEAGSHTIQIMYGCDVGSDGRFLRGYRQDAYDGKDY
IALNEDLRSWTAADMAAQITKRKWEAAHEAEQLRAYLDGTCVEWLRRYL
ENGKETLQRTDPPKTHMTHHPISDHEATLRCWALGFYPAEITLTWQRDG
EDQTQDTELVETRPAGDGTFQKWAAVWPSGEEQRYTCHVQHEGLPKPLT
LRWELSSQPTIPIVGIIAGLVLLGAVITGAVVAAVWMRRKSSDRKGGSY
TQAASSDSAQGSDVSLTACKV

An amino acid sequence of HLA class I—HLA-B is HLA-B07 as shown as SEQ ID NO: 19:

SEQ ID NO: 19
MLVMAPRTVLLLLSAALALTETWAGSHSMRYFYTSVSRPGRGEPRFISV
GYVDDTQFVRFDSDAASPREEPRAPWIEQEGPEYWDRNTQIYKAQAQTD
RESLRNLRGYYNQSEAGSHTLQSMYGCDVGPDGRLLRGHDQYAYDGKDY
IALNEDLRSWTAADTAAQITQRKWEAAREAEQRRAYLEGECVEWLRRYL
ENGKDKLERADPPKTHVTHHPISDHEATLRCWALGFYPAEITLTWQRDG
EDQTQDTELVETRPAGDRTFQKWAAVVVPSGEEQRYTCHVQHEGLPKPL
TLRWEPSSQSTVPIVGIVAGLAVLAVVVIGAVVAAVMCRRKSSGGKGGS
YSQAACSDSAQGSDVSLTA

An amino acid sequence of HLA class I—HLA-B is HLA-B08 as shown as SEQ ID NO: 20:

SEQ ID NO: 20
MLVMAPRTVLLLLSAALALTETWAGSHSMRYFDTA
MSRPGRGEPRFISVGYVDDTQFVRFDSDAASPREE
PRAPWIEQEGPEYWDRNTQIFKTNTQTDRESLRNL
RGYYNQSEAGSHTLQSMYGCDVGPDGRLLRGHNQY
AYDGKDYIALNEDLRSWTAADTAAQITQRKWEAAR
VAEQDRAYLEGTCVEWLRRYLENGKDTLERADPPK
THVTHHPISDHEATLRCWALGFYPAEITLTWQRDG
EDQTQDTELVETRPAGDRTFQKWAAWVPSGEEQRY
TCHVQHEGLPKPLTLRWEPSSQSTVPIVGIVAGLA
VLAVVVIGAVVAAVMCRRKSSGGKGGSYSQAACSD
SAQGSDVSLTA

An illustrative amino acid sequence of HLA class I—HLA-B is HLA-B44 as shown as SEQ ID NO: 21:

SEQ ID NO: 21
MRVTAPRTLLLLLWGAVALTETWAGSHSMRYFYTA
MSRPGRGEPRFITVGYVDDTLFVRFDSDATSPRKE
PRAPWIEQEGPEYWDRETQISKTNTQTYRENLRTA
LRYYNQSEAGSHIIQRMYGCDVGPDGRLLRGYDQD
AYDGKDYIALNEDLSSWTAADTAAQITQRKWEAAR
VAEQDRAYLEGLCVESLRRYLENGKETLQRADPPK
THVTHHPISDHEVTLRCWALGFYPAEITLTWQRDG
EDQTQDTELVETRPAGDRTFQKWAAVVVPSGEEQR
YTCHVQHEGLPKPLTLRWEPSSQSTVPIVGIVAGL
AVLAVVVIGAVVAAVMCRRKSSGGKGGSYSQAACS
DSAQGSDVSLTA

An amino acid sequence of HLA class I—HLA-C is HLA-C01 as shown as SEQ ID NO: 22:

SEQ ID NO: 22
MRVMAPRTLILLLSGALALTETWACSHSMKYFFTS
VSRPGRGEPRFISVGYVDDTQFVRFDSDAASPRGE
PRAPWWEQEGPEYWDRETQKYKRQAQTDRVSLRNL
RGYYNQSEAGSHTLQWMCGCDLGPDGRLLRGYDQY
AYDGKDYIALNEDLRSWTAADTAAQITQRKWEAAR
EAEQRRAYLEGTCVEWLRRYLENGKETLQRAEHPK
THVTHHPVSDHEATLRCWALGFYPAEITLTWQWDG
EDQTQDTELVETRPAGDGTFQKWAAVMVPSGEEQR
YTCHVQHEGLPEPLTLRWEPSSQPTIPIVGIVAGL
AVLAVLAVLGAVVAVVMCRRKSSGGKGGSCSQAAS
SNSAQGSDESLIACKA

The engineered polypeptide of the effector cell of the invention may comprise the extracellular domain of any of SEQ ID Nos 16 to 22, or a variant thereof having at least 80, 85, 90, 95, 98 or 99% identity, provided that the variant maintains ability to assemble with a β2-microglobulin chain and facilitate productive peptide presentation by the MHC class I complex.

The engineered polypeptide may also comprise a transmembrane domain.

The transmembrane domain may be any peptide domain that is capable of inserting into and spanning the cell membrane. A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components). For example, the transmembrane domain may comprise a hydrophobic alpha helix. The transmembrane domain may, for example, be derived from CD8alpha or CD28.

### HLA Class II

In humans the MHC class II protein complex is encoded by the human leukocyte antigen gene complex (HLA). HLAs corresponding to MHC class II are HLA-DP, HLA-DM, HLA-DOA, HLA-DOB, HLA-DQ and HLA-DR.

Activated human T cells express MHC class II molecules of all isotypes (HLA-DR, HLA-DQ, and HLA-DP) on their surface. Expression of MHC class II molecules is found approximately 3 to 5 days after T-cell activation, which is a relative late event compared with the induction of a variety of other effector molecules after T-cell receptor (TCR)-triggering and co-stimulation. Since adoptively transferred immune effectors are expected to be activated at some point after infusion, expression of HLA class II can lead to allo-rejection.

HLA class II molecules are formed as two polypeptide chains: alpha and beta. These are typically highly polymorphic from one individual to another, although some haplotypes are much more common in certain populations than others.

Polypeptides for any haplotype or any combination of haplotypes may be used in the present invention including any of those recited in the table below:

TABLE 3
Exemplary common haplotypes.
HLA-DRB

HLA-DRB03
HLA-DRB15
HLA-DRB04
HLA-DRB07
HLA-DRB01

HLA-DR has very little polymorphism, making it particularly suitable for use in the present invention. In one embodiment, the engineered polypeptide comprises an ectodomain from HLA-DR and an intracellular signalling domain. The ectodomain may be from HLA-DRα or HLA-DRβ.

An amino acid sequence of HLA class II histocompatibility antigen, DR α chain (which has UniProtKB accession number P01903) is shown as SEQ ID NO: 23:

SEQ ID NO: 23
MAISGVPVLGFFIIAVLMSAQESWAIKEEHVIIQA
EFYLNPDQSGEFMFDFDGDEIFHVDMAKKETVWRL
EEFGRFASFEAQGALANIAVDKANLEIMTKRSNYT
PITNVPPEVTVLTNSPVELREPNVLICFIDKFTPP
VVNVTWLRNGKPVTTGVSETVFLPREDHLFRKFHY
LPFLPSTEDVYDCRVEHWGLDEPLLKHWEFDAPSP
LPETTENVVCALGLTVGLVGIIIGTIFIIKGVRKS
NAAERRGPL

Bold underlined=ecotodomain of this HLADRα sequence corresponds to amino acid positions 26-216 of the sequence.

The engineered polypeptide may comprise an ectodomain from HLA-DRα as set forth SEQ ID NO: 23 (such as from about amino acid 26 to about amino acid 216 of SEQ ID NO: 23) or a variant thereof having at least 80, 85, 90, 95, 98 or 99% sequence identity, provided that the variant maintains ability to assemble with a β chain and facilitate productive peptide presentation by the MHC class II complex.

An amino acid sequence of HLA class II histocompatibility antigen, DR β chain (which has UniProtKB accession number Q04826) is shown as SEQ ID NO: 24:

SEQ ID NO: 24
MRVTAPRTLLLLLWGAVALTETWAGSHSMRYFHTS
VSRPGRGEPRFITVGYVDDTLFVRFDSDATSPRKE
PRAPWIEQEGPEYWDRETQISKTNTQTYRESLRNL
RGYYNQSEAGSHTLQSMYGCDVGPDGRLLRGHNQY
AYDGKDYIALNEDLRSWTAADTAAQITQRKWEAAR
VAEQLRAYLEGECVEWLRRYLENGKETLQRADPPK
THVTHHPISDHEATLRCWALGFYPAEITLTWQRDG
EDQTQDTELVETRPAGDRTFQKWAAVVVPSGEEQR
YTCHVQHEGLPKPLTLRWEPSSQSTVPIVGIVAGL
AVLAVVVIGAVVAAVMCRRKSSGGKGGSYSQAACS
DSAQGSDVSLTA

Bold underlined=the ecotodomain of this HLA-DRβ sequence and corresponds to amino acid positions 25-308 of the sequence.

The engineered polypeptide may comprise an ectodomain from HLA-DRβ as set forth SEQ ID NO: 24 (such as from about amino acid 25 to about amino acid 308 of SEQ ID NO: 24) or a variant thereof having at least 80, 85, 90, 95, 98 or 99% sequence identity, provided that the variant maintains ability to assemble with an α chain and facilitate productive peptide presentation by the MHC class II complex.

HLA-DP and HLA-DQ have polymorphic α and β chains. Therefore, one can select common HLA-DP or HLA-DQ α or β chain and restrict allogeneic production only from recipients with that haplotype. Suitably, the recipient may be homozygous for that haplotype. Wherein the recipient is not homozygous for the haplotype, two HLA-DP and two HLA-DQ (optionally in combination with HLA-DR e.g. HLA-DRα) may be used.

An amino acid sequence of HLA class II histocompatibility antigen, DP (which has UniProtKB accession number Q30058) is shown as SEQ ID NO: 25:

SEQ ID NO. 25
MVLQVSAAPRTVALTALLMVLLTSVVQGRATPENY
VHQLRQECYGFNGTQRFLESYIYNREEFVRFDSDV
GEFRAVTELGRPDEDYWNSQKDILEEERAVPDRVC
RRNYELDEAVTLQRRVQPKVNVSPSKKGPLQHHNL
LVCHVTDFYPSSIQVRWFLNGQEETAGWSTNLIRN
GDWTFQILVMLEMTPQQGDVYICQVEHTSLDSPVT
VEWKAQSDSAQSKTLTGAGGFVLGLIICGVGIFMH
RRSKKVQRGSA

Italics=the transmembrane region and corresponds to amino acid positions 225 to 244
Bold underlined=the ecotodomain of this HLA-DP sequence and corresponds to amino acid positions 29-224 of the sequence

The engineered polypeptide may comprise an ectodomain from HLA-DP as set forth SEQ ID NO: 25 (such as from about amino acid 29 to about amino acid 224 of SEQ ID NO: 25) or a variant thereof having at least 80, 85, 90, 95, 98 or 99% sequence identity, provided that the variant maintains ability to assemble and facilitate productive peptide presentation by the MHC class II complex.

An amino acid sequence of HLA class II histocompatibility antigen, DQ (which has UniProtKB accession number 019764) is shown as SEQ ID NO: 26:

SEQ ID NO. 26
SWKKALRIPGDLRVATVTLMLAMLSSLLAEGRDSP
EDFVYQFKGLCYFTNGTERVRLVTRYIYNREEYAR
FDSDVGVYRAVTPQGRPVAEYWNSQKEVLERTRAE
LDTVCRHNYEVGYRGILQRRVEPTVTISPSRTEAL
NHHNLLVCSVTDFYPGQIKVQWFRNDQEETAGVVS
TPLIRNGDWTFQILMLEMTPQRGDVYTCHVEHPSL
QSPITVEWRAQSESAQSKMLSGVGGFVLGLIFLGL
GLII

Italics=the transmembrane region and corresponds to amino acid positions 229-249
Bold underlined=the ecotodomain of this HLA-DQ sequence and corresponds to amino acid positions 32-228 of the sequence.

The engineered polypeptide may comprise an ectodomain from HLA-DQ as set forth SEQ ID NO: 26 (such as from about amino acid 32 to about amino acid 228 of SEQ ID NO: 26) or a variant thereof having at least 80, 85, 90, 95, 98 or 99% sequence identity, provided that the variant maintains ability to assemble and facilitate productive peptide presentation by the MHC class II complex.

The engineered polypeptide may comprise the extracellular domain from any of SEQ ID No. 23 to 26. The engineered polypeptide may also comprise a transmembrane domain, as explained above.

The sequences of MHC polypeptides are provided in the ImMunoGeneTics (IMGT) database (Lefranc, M.-P. et al., Nucleic Acids Res., 27:209-212 (1999); doi: 10.1093/nar/27.1.209).

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

As used herein, “capable of co-localizing an MHC class I polypeptide or MHC class II polypeptide with an intracellular signalling domain within the cell” means that, when a target T-cell binds to a peptide/MHC complex on an effector immune cell of the present invention, the polypeptide co-localizes the MHC class I polypeptide or MHC class II polypeptide with the intracellular signalling domain such that the intracellular signalling domain transmits an activating signal in the effector immune cell of the present invention.

### CD79

CD79 is comprised of two chains, CD79α and CD79β which form a heterodimer on the surface of B cells. CD79α a/β assemble with membrane-bound immunoglobulin forming a complex with the B-cell receptor (BCR). CD79α and CD79β are members of the immunoglobulin superfamily and contain ITAM signalling motifs which enable B-cell signalling in response to cognate antigen recognition by the BCR.

CD79α and CD79β also associate with HLA class II, which allows HLA class II to signal through CD79 in an analogous way to membrane-bound immunoglobulin (Lang, P. et al. Science 291, 1537-1540 (2001) and Jin, L. et al. Immunol. Lett. 116, 184-194 (2008).

In one aspect, the present invention provides a cell which comprises; (i) a chimeric antigen receptor (CAR) or a transgenic T-cell receptor (TCR); and (ii) at least one polypeptide capable of co-localizing an MHC class I polypeptide or an MHC class II polypeptide with an intracellular signalling domain within the cell; wherein the at least one polypeptide capable of co-localizing the MHC class I polypeptide or MHC class II polypeptide with the intracellular signalling domain is CD79 or a variant thereof.

The cell may comprise an engineered polypeptide which comprises CD79α or CD79β linked to an intracellular signalling domain. The cell may comprise two engineered polypeptides: one which comprises CD79α linked to an intracellular signalling domain; and one which comprises CD79β linked to an intracellular signalling domain.

The amino acid sequence of human CD79 α (which has UniProtKB accession number P11912) is shown as SEQ ID NO: 27:

SEQ ID NO. 27
MPGGPGVLQALPATIFLLFLLSAVYLGPGCQALWM
HKVPASLMVSLGEDAHFQCPHNSSNNANVTWWRVL
HGNYTWPPEFLGPGEDPNGTLIIQNVNKSHGGIYV
CRVQEGNESYQQSCGTYLRVRQPPPRPFLDMGEGT
KNRIITAEGIILLFCAVVPGTLLLFRKRWQNEKLG
LDAGDEYEDENLYEGLNLDDCSMYEDISRGLQGTY
QDVGSLNIGDVQLEKP

Underlined=signal peptide (amino acids 1-32)
Bold=extracellular (amino acids 33-143)
No formatting=transmembrane domain (amino acids 144-165)
Italics=cytoplasmic domain (amino acids 166-226)

A CD79 α sequence for use in the present invention may comprise the sequence shown as SEQ ID NO: 27 or a variant thereof having at least 80, 85, 90, 95, 98 or 99% sequence identity, provided that the variant maintains ability to assemble with HLA class I and/or HLA class II and facilitate signalling.

The engineered polypeptide may comprise an ectodomain of CD79α, a transmembrane domain and an intracellular signalling domain. The engineered polypeptide may comprise an ectodomain of CD79α which corresponds to about amino acid 33 to about amino acid 143 of SEQ ID NO. 27.

The engineered polypeptide may comprise a transmembrane domain of CD79α which corresponds to about amino acid 144 to about amino acid 165 of SEQ ID NO. 27.

The engineered polypeptide may comprise an intracellular signalling domain of CD79α which corresponds to about amino acid 166 to about amino acid 226 of SEQ ID NO. 27.

The amino acid sequence of human CD79 β (which has UniProtKB accession number P40259) is shown as SEQ ID NO: 28:

SEQ ID NO. 28
MARLALSPVPSHWMVALLLLLSAEPVPAARSEDRY
RNPKGSACSRIWQSPRFIARKRGFTVKMHCYMNSA
SGNVSWLWKQEMDENPQQLKLEKGRMEESQNESLA
TLTIQGIRFEDNGIYFCQQKCNNTSEVYQGCGTEL
RVMGFSTLAQLKQRNTLKDGIIMIQTLLIILFIIV
PIFLLLDKDDSKAGMEEDHTYEGLDIDQTATYEDI
VTLRTGEVKWSVGEHPGQE

Underlined=signal peptide (amino acids 1-28)
Bold=extracellular (amino acids 29-159)
No formatting=transmembrane domain (amino acids 160-180)
Italics=cytoplasmic (amino acids 181-229)

A CD79 β sequence for use in the present invention may comprise the sequence shown as SEQ ID NO: 8 or a variant thereof having at least 80, 85, 90, 95, 98 or 99% sequence identity, provided that the variant maintains ability to assemble with HLA class I and/or HLA class II and facilitate signalling.

The engineered polypeptide may comprise an ectodomain of CD79β, a transmembrane domain and an intracellular signalling domain. The engineered polypeptide may comprise an ectodomain of CD79β which corresponds to about amino acid 29 to about amino acid 159 of SEQ ID NO. 28.

The engineered polypeptide may comprise a transmembrane domain of CD79 β which corresponds to about amino acid 160 to about amino acid 180 of SEQ ID NO. 28.

The engineered polypeptide may comprise an intracellular signalling domain of CD79 β which corresponds to about amino acid 181 to about amino acid 229 of SEQ ID NO. 28.

The effector immune cell may express two engineered polypeptides: one comprising the extracellular domain of CD79α and one comprising the extracellular domain of CD79β.

### CD3 Linked Polypeptide

The effector immune cell may comprise:

(i) a chimeric antigen receptor (CAR) or a transgenic T-cell receptor (TCR); and
(ii) an engineered polypeptide which comprises an MHC class I polypeptide or MHC class II polypeptide linked to linked to a component of the CD3/TCR complex.

CD3 is a T-cell co-receptor that is involved in the activation of both cytotoxic T-cells and T-helper cells. It is formed of a protein complex composed of four distinct chains. As used herein, the term “CD3 complex” also includes the CD3 ζ-chain. In mammals, the complex contains a CD3γ chain, a CD3δ chain, and two CD3ε chains. These chains associate with the TCR to generate a TCR complex which is capable of producing an activation signal in T lymphocytes.

The CD3ζ, CD3γ, CD3δ, and CD3ε chains are highly related cell-surface proteins of the immunoglobulin superfamily containing a single extracellular immunoglobulin domain. The transmembrane region of the CD3 chains contain a number of aspartate residues are negatively charged, a characteristic that allows these chains to associate with the positively charged TCR chains. The intracellular tails of the CD3 molecules contain a single conserved motif known as an immunoreceptor tyrosine-based activation motif (ITAM), which is involved in TCR signalling.

The polypeptide linked to a component of the TCR complex is capable of assembling and facilitating productive peptide presentation by the MHC class I or MHC class II complex at the cell surface. In addition, the TCR/CD3 component is able to assemble with the TCR/CD3 complex. Hence, binding of a TCR to the peptide/MHC complex comprising the polypeptide linked to a component of the TCR complex will trigger signalling through the CD3/TCR complex.

The polypeptide may be linked to the TCR or a component of the CD3 complex. The polypeptide may be linked to an engineered TCR polypeptide which lacks a variable domain.

The engineered polypeptide may be linked to a component of the CD3 complex, for example selected from CD3-zeta, CD3-epsilon, CD3-gamma and CD3-delta.

Examples of human CD3ζ, CD3γ, CD3δ and CD3ε amino acid sequences are shown as SEQ ID NO: 29-32, respectively.

(CD3ζ-amino acids 1-21 provide a signal
peptide which may be excluded, the
transmembrane domain is underlined)
SEQ ID NO: 29
MKWKALFTAAILQAQLPITEAQSFGLLDPKLCYLL
DGILFIYGVILTALFLRVKFSRSADAPAYQQGQNQ
LYNELNLGRREEYDVLDKRRGRDPEMGGKPQRRKN
PQEGLYNELQKDKMAEAYSEIGMKGERRRGKGHDG
LYQGLSTATKDTYDALHMQALPPR
(CD3γ-amino acids 1-22 provide a signal
peptide which may be excluded)
SEQ ID NO: 30
MEQGKGLAVLILAIILLQGTLAQSIKGNHLVKVYD
YQEDGSVLLTCDAEAKNITWFKDGKMIGFLTEDKK
KWNLGSNAKDPRGMYQCKGSQNKSKPLQVYYRMCQ
NCIELNAATISGFLFAEIVSIFVLAVGVYFIAGQD
GVRQSRASDKQTLLPNDQLYQPLKDREDDQYSHLQ
GNQLRRN
(CD3δ-amino acids 1-21 provide a signal
peptide which may be excluded)
SEQ ID NO: 31
MEHSTFLSGLVLATLLSQVSPFKIPIEELEDRVFV
NCNTSITWVEGTVGTLLSDITRLDLGKRILDPRGI
YRCNGTDIYKDKESTVQVHYRMCQSCVELDPATVA
GIIVTDVIATLLLALGVFCFAGHETGRLSGAADTQ
ALLRNDQVYQPLRDRDDAQYSHLGGNWARNK
(CD3ϵ-amino acids 1-22 provide a signal
peptide which may be excluded)
SEQ ID NO: 32
MQSGTHWRVLGLCLLSVGVWGQDGNEEMGGITQTP
YKVSISGTTVILTCPQYPGSEILWQHNDKNIGGDE
DDKNIGSDEDHLSLKEFSELEQSGYYVCYPRGSKP
EDANFYLYLRARVCENCMEMDVMSVATIVIVDICI
TGGLLLLVYYWSKNRKAKAKPVTRGAGAGGRQRGQ
NKERPPPVPNPDYEPIRKGQRDLYSGLNQRRI

The MHC class I/MHC class II or B2M polypeptide may be linked to the CD3 component by any suitable means. For example, the polypeptide may be fused to the component of the CD3 complex by a linker peptide.

Suitable linker peptides are known in the art. For example, a range of suitable linker peptides are described by Chen et al. (Adv Drug Deliv Rev. 2013 Oct. 15; 65(10): 1357-1369—see Table 3 in particular).

A suitable linker is an (SGGGG)n (SEQ ID NO: 33), which comprises one or more copies of SEQ ID NO: 33. For example, a suitable linker peptide is shown as SEQ ID NO: 34.

SEQ ID NO: 34
SGGGGSGGGGSGGGGS

The polypeptide may be linked to the ectodomain of the component of the CD3 complex. It may be linked to the N-terminus of the component of the CD3 complex.

An illustrative polypeptide for use in the present invention is shown as SEQ ID NO: 35.

METDTLLLVLLLVPGSTGIKEEHVIIQAEFYLNPD
QSGEFMFDFDGDEIFHVDMAKKETVWRLEEFGRFA
SFEAQGALANIAVDKANLEIMTKRSNYTPITNVPP
EVTVLTNSPVELREPNVLICFIDKFTPPVVNVTWL
RNGKPVTTGVSETVFLPREDHLFRKFHYLPFLPST
EDVYDCRVEHWGLDEPLLKHWEFDAPSPLPETTEN
VVCALGLTVGLVGIIIGTIFIIRVKFSRSADAPAY
QQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPRA

This polypeptide sequence comprises an ectodomain from HLA-DRα, a transmembrane domain an intracellular CD3-ζ endodomain.

An illustrative polypeptide for use in the present invention is shown as SEQ ID NO: 36.

METDTLLLVLLLVPGSTGIKEEHVIIQAEFYLNPD
QSGEFMFDFDGDEIFHVDMAKKETVWRLEEFGRFA
SFEAQGALANIAVDKANLEIMTKRSNYTPITNVPP
EVTVLTNSPVELREPNVLICFIDKFTPPVVNVTWL
RNGKPVTTGVSETVFLPREDHLFRKFHYLPFLPST
EDVYDCRVEHWGLDEPLLKHWEFDAPSPLPETTEN
VVCALGLTVGLVGIIIGTIFIIKRGRKKLLYIFKQ
PFMRPVQTTQEEDGCSCRFPEEEEGGCELRVKFSR
SADAPAYQQGQNQLYNELNLGRREEYDVLDKRRGR
DPEMGGKPRRKNPQEGLYNELQKDKMAEAYSEIGM
KGERRRGKGHDGLYQGLSTATKDTYDALHMQALPP
RA

This polypeptide sequence comprises an ectodomain from HLA-DRα, a transmembrane domain, a 41BB endodomain and an intracellular CD3-ζ endodomain.

An illustrative polypeptide for use in the present invention is shown as SEQ ID NO: 37.

METDTLLLWVLLLVWPGSTGIKEEHVIIQAEFYLN
PDQSGEFMFDFDGDEIFHVDMAKKETVWRLEEFGR
FASFEAQGALANIAVDKANLEIMTKRSNYTPITNV
PPEVTVLTNSPVELREPNVLICFIDKFTPPVVNVT
WLRNGKPVTTGVSETVFLPREDHLFRKFHYLPFLP
STEDVYDCRVEHWGLDEPLLKHWEFDAPSPLPETT
ENVVCALGLTVGLVGIIIGTIFIIRSKRSRLLHSD
YMNMTPRRPGPTRKHYQPYAPPRDFAAYRSRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PRA

This polypeptide sequence comprises an ectodomain from HLA-DRα, a transmembrane domain, a CD28 endodomain and an intracellular CD3-ζ endodomain.

An illustrative polypeptide for use in the present invention is shown as SEQ ID NO: 38.

METDTLLLWVLLLVPGSTGLWMHKVPASLMVSLGE
DAHFQCPHNSSNNANVTWWRVLHGNYTWPPEFLGP
GEDPNGTLIIQNVNKSHGGIYVCRVQEGNESYQQS
CGTYLRVRQPPPRPFLDMGEGTKNRIITAEGIILL
FCAVVPGTLLLFKRGRKKLLYIFKQPFMRPVQTTQ
EEDGCSCRFPEEEEGGCELRKRWQNEKLGLDAGDE
YEDENLYEGLNLDDCSMYEDISRGLQGTYQDVGSL
NIGDVQLEKP

This polypeptide sequence comprises an ectodomain from CD79α, a 41BB domain and an endodomain from CD79.

An illustrative polypeptide for use in the present invention is shown as SEQ ID NO: 39.

METDTLLLVLLLVPGSTGARSEDRYRNPKGSACSRIWQSPRFIARKRGF
TVKMHCYMNSASGNVSWLWKQEMDENPQQLKLEKGRMEESQNESLATLT
IQGIRFEDNGIYFCQQKCNNTSEVYQGCGTELRVMGFSTLAQLKQRNTL
KDGIIMIQTLLIILFIIVPIFLLRSKRSRLLHSDYMNMTPRRPGPTRKH
YQPYAPPRDFAAYRSLDKDDSKAGMEEDHTYEGLDIDQTATYEDIVTLR
TGEVKWSVGEHPGQE

This polypeptide sequence comprises an ectodomain from CD79β, a CD28 domain and an endodomain from CD79.

An illustrative polypeptide for use in the present invention is shown as SEQ ID NO: 40.

METDTLLLVLLLVPGSTGLWMHKVPASLMVSLGEDAHFQCPHNSSNNAN
VTWWRVLHGNYTWPPEFLGPGEDPNGTLIIQNVNKSHGGIYVCRVQEGN
ESYQQSCGTYLRVRQPPPRPFLDMGEGTKNRIITAEGIILLFCAVVPGT
LLLFRSKRSRLLHSDYMNMTPRRPGPTRKHYQPYAPPRDFAAYRSRKRW
QNEKLGLDAGDEYEDENLYEGLNLDDCSMYEDISRGLQGTYQDVGSLNI
GDVQLEKP

This polypeptide sequence comprises an ectodomain from CD79α, a CD28 domain and an endodomain from CD79.

An illustrative polypeptide for use in the present invention is shown as SEQ ID NO: 41.

METDTLLLVLLLVPGSTGARSEDRYRNPKGSACSRIWQSPRFIARKRGF
TVKMHCYMNSASGNVSWLWKQEMDENPQQLKLEKGRMEESQNESLATLT
IQGIRFEDNGIYFCQQKCNNTSEVYQGCGTELRVMGFSTLAQLKQRNTL
KDGIIMIQTLLIILFIIVPIFLLKRGRKKLLYIFKQPFMRPVQTTQEED
GCSCRFPEEEEGGCELLDKDDSKAGMEEDHTYEGLDIDQTATYEDIVTL
RTGEVKWSVGEHPGQE

This polypeptide sequence comprises an ectodomain from CD79β, a 41BB domain and an endodomain from CD79.

An illustrative polypeptide for use in the present invention is shown as SEQ ID NO: 42.

METDTLLLWVLLLVPGSTGLWMHKVPASLMVSLGEDAHFQCPHNSSNNA
NVTWWRVLHGNYTWPPEFLGPGEDPNGTLIIQNVNKSHGGIYVCRVQEG
NESYQQSCGTYLRVRQPPPRPFLDMGEGTKNRIITAEGIILLFCAVVPG
TLLLFKRGRKKLLYIFKQPFMRPVQTTQEEDGCSCRFPEEEEGGCELRV
KFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDT
YDALHMQALPPRA

This polypeptide sequence comprises an ectodomain from CD79α, a 41BB domain and a CD3-zeta domain.

An illustrative polypeptide for use in the present invention is shown as SEQ ID NO: 43.

METDTLLLVLLLVPGSTGARSEDRYRNPKGSACSRIWQSPRFIARKRGF
TVKMHCYMNSASGNVSWLWKQEMDENPQQLKLEKGRMEESQNESLATLT
IQGIRFEDNGIYFCQQKCNNTSEVYQGCGTELRVMGFSTLAQLKQRNTL
KDIITAEGIILLFCAVVPGTLLLFKRGRKKLLYIFKQPFMRPVQTTQEE
DGCSCRFPEEEEGGCELRVKFSRSADAPAYQQGQNQLYNELNLGRREEY
DVLDKRRGRDPEMGGKPRRKNPQEGLYNELQKDKMAEAYSEIGMKGERR
RGKGHDGLYQGLSTATKDTYDALHMQALPPRA

This polypeptide sequence comprises an ectodomain from CD79β, a 41BB domain and a CD3-zeta domain.

A polypeptide sequence for use in the present invention may comprise the sequence shown as SEQ ID NO: 35-43 or a variant thereof having at least 80, 85, 90, 95, 98 or 99% sequence identity, provided that the variant maintains ability to assemble and facilitate productive peptide presentation by the MHC class II complex at the surface of the cell and transmit an activating signal following binding of a TCR to the peptide/MHC complex comprising the polypeptide.

### Intracellular Signalling Domain

The present invention involves providing at least one polypeptide capable of co-localizing an MHC class I polypeptide or MHC class II polypeptide with an intracellular signalling domain within the cell.

The engineered polypeptide of the invention may comprise an intracellular signalling domain

An intracellular signalling domain as used herein refers to a signal-transmission portion of an endodomain.

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

Preferably, the intracellular signalling domain component comprises, consists essentially of, or consists of the CD3-ζ endodomain, which contains three ITAMs. Classically, the CD3-ζ endodomain transmits an activation signal to the T cell after antigen is bound. However, in the context of the present invention, the CD3-ζ endodomain transmits an activation signal to the effector cell after its MHC complex interacts with a TCR on a neighbouring T cell.

The intracellular signalling domain may comprise additional co-stimulatory signalling. For example, 4-1BB (also known as CD137) can be used with CD3-ζ, or CD28 and OX40 can be used with CD3-ζ to transmit a proliferative/survival signal.

Accordingly, intracellular signalling domain may comprise the CD3-ζ endodomain alone, the CD3-ζ endodomain in combination with one or more co-stimulatory domains selected from 4-1BB, CD28 or OX40 endodomain, and/or a combination of some or all of 4-1BB, CD28 or OX40.

The endodomain may comprise one or more of the following: an ICOS endodomain, a CD2 endodomain, a CD27 endodomain, or a CD40 endodomain.

The endodomain may comprise the sequence shown as SEQ ID NO: 44-47 or a variant thereof having at least 80, 85, 90, 95, 98 or 99% sequence identity, provided that the variant sequence retains the capacity to transmit an activating signal to the cell.

-CD3-G endodomain
SEQ ID NO: 44
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
-4-1BB and CD3-ζ endodomains
SEQ ID NO: 45
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCELRVKFSRSADAPAYQQGQNQLYNELNLGRREEYDVLDKRR
GRDPEMGGKPQRRKNPQEGLYNELQKDKMAEAYSEIGMKGERRRGKGHD
GLYQGLSTATKDTYDALHMQALPPR
-CD28 and CD3-ζ endodomains
SEQ ID NO: 46
SKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQ
ALPPR
-CD28, OX40 and CD3-ζ endodomains
SEQ ID NO: 47
SKRSRLLHSDYMNMTPRRPGPTRKHYQPYAPPRDFAAYRSRDQRLPPDA
HKPPGGGSFRTPIQEEQADAHSTLAKIRVKFSRSADAPAYQQGQNQLYN
ELNLGRREEYDVLDKRRGRDPEMGGKPRRKNPQEGLYNELQKDKMAEAY
SEIGMKGERRRGKGHDGLYQGLSTATKDTYDALHMQALPPR

### Antigen-Binding Domain Linked to Signalling Domain

The engineered polypeptide of the present invention may comprise a binding domain which binds to an MHC class I polypeptide; an MHC class II polypeptide or β2 microglobulin, linked to an intracellular signalling domain.

The binding domain may be or comprise and antibody or antibody-like molecule.

The term “antibody”, as used herein, refers to a polypeptide having an antigen binding site which comprises at least one complementarity determining region or CDR. The antibody may comprise 3 CDRs and have an antigen binding site which is equivalent to that of a single domain antibody (dAb), heavy chain antibody (VHH) or a nanobody. The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen.

A full-length antibody or immunoglobulin typically consists of four polypeptides: two identical copies of a heavy (H) chain polypeptide and two identical copies of a light (L) chain polypeptide. Each of the heavy chains contains one N terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. They are characterised by the same general structure constituted by relatively preserved regions called frameworks (FR) joined by three hyper-variable regions called complementarity determining regions (CDR). The term “complementarity determining region” or “CDR”, as used herein, refers to the region within an antibody that complements an antigen's shape. Thus, CDRs determine the protein's affinity and specificity for specific antigens. The CDRs of the two chains of each pair are aligned by the framework regions, acquiring the function of binding a specific epitope. Consequently, in the case of VH and VL domains both the heavy chain and the light chain are characterised by three CDRs, respectively CDRH1, CDRH2, CDRH3 and CDRL1, CDRL2, CDRL3.

The engineered polypeptide of the present invention may comprise a full-length antibody or an antigen-binding fragment thereof.

A full length antibody may, for example be an IgG, an IgM, an IgA, an IgD or an IgE.

An “antibody fragment” refers to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, a Fab fragment, a F(ab′)2 fragment, an Fv fragment, a single chain Fv (scFv), a domain antibody (dAb or VH), a single domain antibody (sdAb), a VHH, a nanobody, a diabody, a triabody, a trimerbody, and a monobody.

The engineered polypeptide of the invention may comprise an antigen-binding domain which is based on a non-immunoglobulin scaffold. These antibody-binding domains are also called antibody mimetics. Non-limiting examples of non-immunoglobulin antigen-binding domains include an affibody, a fibronectin artificial antibody scaffold, an anticalin, an affilin, a DARPin, a VNAR, an Body, an affimer, a fynomeran, abdurin/nanoantibody, a centyrin, an alphabody, a nanofitin, and a D domain.

Several antibodies have been described which specifically bind MHC class I or MHC class II.

For example, WO05/023299, which is incorporated by reference, describes antibodies which bind MHC class II antigens, in particular antibodies against the HLA-DR alpha chain. Table 1 of that document contains the sequence characteristics of clones MS-GPC-1 (scFv-17), MS-GPC-6 (scFv-8A), MS-GPC-8 (scFv-B8) and MS-GPC-10 (scFv-E6) and FIG. 15 gives the VH and VL sequences for MS-GPC-1; MS-GPC-6; MS-GPC-8; MS-GPC-10; MS-GPC-8-6; MS-GPC-8-10; MS-GPC-8-17; MS-GPC-8-27; MS-GPC-8-6-13; MS-GPC-8-10-57; MS-GPC-8-27-41; MS-GPC-8-1; MS-GPC-8-9; MS-GPC-8-18; MS-GPC-8-6-2; MS-GPC-8-6-19; MS-GPC-8-6-27; MS-GPC-8-6-45; MS-GPC-8-6-47; MS-GPC-8-27-7; and MS-GPC-8-27-10.

The engineered polypeptide may comprise an MHC class II binding domain comprising one of these pairs of VH and VL sequences. In particular, engineered polypeptide may comprise an MHC class II binding domain based on the binder MS-GPC-8.

Andris et al (1995 Mol Immunol 32:14-15) describe six antibodies specific for human HLA class I and class II antigens including an antibody against HLA-DQ beta chain having the antibody clone name anti-HLAII/DQB1-MP1.

Watkins et al (2000 Tissue Antigens 55: 219-28) describe the isolation and characterisation of human monoclonal HLA-A2 antibodies. The antibody clones include: anti-HLA-A2/A28-3PF12, anti-HLA-A2/A28-3PC4 and anti-HLA-A2/A28-3PB2.

The engineered polypeptide of the present invention may comprise an MHC class I or MHC class II binding domain derived from any of these antibodies.

The engineered polypeptide may comprise a short flexible linker to introduce a chain-break. A chain break separate two distinct domains but allows orientation in different angles. Such sequences include the sequence SDP, and the sequence SGGGSDP (SEQ ID NO: 48).

The linker may comprise a serine-glycine linker, such as SGGGGS (SEQ ID NO: 49).

The engineered polypeptide may comprise a transmembrane domain, as defined above. The engineered polypeptide may, for example, comprise the transmembrane domains of CD8-alpha or CD28.

The engineered polypeptide comprises an intracellular signalling domain, as defined above. The engineered polypeptide may, for example, comprise the CD3ζ endodomain.

The engineered polypeptide may have the general structure:

MHC class I or II binding domain-transmembrane domain-intracellular signalling domain, or
MHC class I or II binding domain-linker-transmembrane domain-intracellular signalling domain

### CD4/CD8 Fusion Proteins

The engineered polypeptide of the present invention may comprise the MHC class II-binding domain of CD4 linked to an intracellular signalling domain, or MHC class I-binding domain of CD8 linked to an intracellular signalling domain.

CD4 and CD8 are co-receptors of the T cell receptor (TCR) and assists T cells in communicating with antigen-presenting cells.

CD4 (cluster of differentiation 4) is a glycoprotein found on the surface of immune cells such as T helper cells, monocytes, macrophages, and dendritic cells. CD4 is a member of the immunoglobulin superfamily, having four immunoglobulin domains (D1 to D4) that are exposed on the extracellular surface of the cell:
D1 which resembles an immunoglobulin variable (IgV) domain; and
D2, D3 and D4 which resemble immunoglobulin constant (IgC) domains.

The immunoglobulin variable (IgV) domain of D1 adopts an immunoglobulin-like β-sandwich fold with seven p-strands in 2 β-sheets. CD4 interacts with the β2-domain of MHC class II molecules through its D1 domain. T cells displaying CD4 molecules on their surface, therefore, are specific for antigens presented by MHC II, i.e. they are MHC class II-restricted.

The short cytoplasmic/intracellular tail (C) of CD4 contains a sequence of amino acids that allow it to recruit and interact with the tyrosine kinase Lck. When the extracellular D1 domain of CD4 binds to the β2 region of MHC class II, the resulting close proximity between the TCR complex and CD4 allows the tyrosine kinase Lck bound to the cytoplasmic tail of CD4 to phosphorylate tyrosine residues of immunoreceptor tyrosine activation motifs (ITAMs) on the cytoplasmic domains of CD3 to amplify the signal generated by the TCR. Phosphorylated ITAMs on CD3 recruit and activate SH2 domain-containing protein tyrosine kinases (PTK), such as ZAP70, to further mediate downstream signalling through tyrosine phosphorylation. These signals lead to the activation of transcription factors, including NF-κB, NFAT, AP-1, to promote T cell activation.

The amino acid sequence for human CD4 is available from UniProt, Accession No. P01730. The engineered polypeptide of the present invention may comprise the D1 domain of CD4, which has the sequence shown as SEQ ID No. 50. The positions of Gln40 and Thr45 are shown in bold and underlined.

(CD4 D1 domain)
SEQ ID No. 50
KKVVLGKKGDTVELTCTASQKKSIQFHWKNSNQIKILGNQGSFLTKGPS
KLNDRADSRRSLWDQGNFPLIIKNLKIEDSDTYICEVEDQKEEVQLLVF
GL

The engineered polypeptide may comprise a variant D1 domain of CD4 comprising one or more amino acid mutations which increase the its binding affinity for the β2 region of MHC class II compared to the wild-type D1 domain.

For example, Wang et al (2011, PNAS 108:15960-15965) describe the affinity maturation of human CD4 by yeast surface display to increase the affinity of CD4 for HLA-DR1. It was found that a CD4 variant bearing the substitution mutations Gln40Tyr and Thr45Trp bound to HLA-DR1 with KD=8.8 μM compares with >400 μM for wild-type CD4.

The engineered polypeptide may comprise a variant D1 domain of CD4 comprising amino acid mutation(s) at position Gln40 and/or Thr45 with reference to the sequence shown as SEQ ID No. 50.

The engineered polypeptide may comprise a variant D1 domain of CD4 comprising amino acid substitution(s) Gln40Tyr and/or Thr45Trp with reference to the sequence shown as SEQ ID No. 50.

CD8 (cluster of differentiation 8) co-receptor is predominantly expressed on the surface of cytotoxic T cells, but can also be found on natural killer cells, cortical thymocytes, and dendritic cells. There are two isoforms CD8, alpha and beta, each encoded by a different gene.

To function, CD8 forms a dimer, consisting of a pair of CD8 chains. The most common form of CD8 is composed of a CD8-α and CD8-β chain, but homodimers of the CD8-α chain are also expressed on some cells. CD8-α and CD8-β are both members of the immunoglobulin superfamily having an immunoglobulin variable (IgV)-like extracellular domain connected to the membrane by a thin stalk, and an intracellular tail.

The extracellular IgV-like domain of CD8-α interacts with the α3 portion of the Class I MHC molecule. The main recognition site is a flexible loop at the α3 domain of an MHC molecule located between residues 223 and 229. Binding of CD8-α to MHC class I keeps the T cell receptor of the cytotoxic T cell and the target cell bound closely together during antigen-specific activation. The cytoplasmic tails of the CD8 co-receptor interact with Lck (lymphocyte-specific protein tyrosine kinase). Once the T cell receptor binds its specific antigen, Lck phosphorylates the cytoplasmic CD3 and ζ-chains of the TCR complex which initiates a cascade of phosphorylation eventually leading to activation of transcription factors like NFAT, NF-κB, and AP-1.

The engineered polypeptide of the present invention may comprise the IgV-like domain from CD8-α.

The amino acid sequence for human CD8α is available from UniProt, Accession No. P01732. The engineered polypeptide of the present invention may comprise the Ig-like V-type domain of CD8, which comprises amino acid residues 22-135 of this sequence and has the sequence shown as SEQ ID No. 51.

(CD8α Ig-like V-type domain)
SEQ ID No. 51
SQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALS
NSIMYFSHFVPVFLPA

The engineered polypeptide may comprise a variant CD8α Ig-like V-type domain comprising one or more amino acid mutations which increase the its binding affinity for the α3 portion of a Class I MHC molecule compared to the wild-type CD8α domain.

For example, high affinity mutants of CD8α may be generated and characterised using the in vitor evolution method described by Wang et al (2011, PNAS 108:15960-15965).

The engineered polypeptide may comprise a dimeric form of CD8. Devine et al (1999, J. Immunol. 162:846-851) describe a molecule which comprises two CD8α Ig domains linked via the carboxyl terminal of one to the amino terminal of the other by means of a peptide spacer. A peptide spacer of 20 amino acids of 4 repeating units of GGGGS (SEQ ID No. 52) was used to allow the 2 IG-like domains to adopt the correct confirmation.

The engineered polypeptide may comprise a CD8αα homodimer, as described in Devine et al 1999. The CD8αα homodimer may have the sequence shown as SEQ ID No. 53.

(CD8αα homodimer)
SEQ ID No. 53
SQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALS
NSIMYFSHFVPVFLPAGGGGSGGGGSGGGGSGGGGSSQFRVSPLDRTWN
LGETVELKCQVLLSNPTSGCSWLFQPRGAAASPTFLLYLSQNKPKAAEG
LDTQRFSGKRLGDTFVLTLSDFRRENEGYYFCSALSNSIMYFSHFVPVF
LPA

The engineered polypeptide may comprise a CD8αβ heterodimer. For example, the engineered polypeptide may comprise an CD8α Ig-like V-type domain having the sequence shown as SEQ ID No. 51 joined to a an CD8P Ig-like V-type domain by a peptide spacer. The peptide spacer may be from 10 to 20, for example between 15 and 25 amino acids in length. The peptide spacer may be approximately 20 amino acids in length. The peptide spacer may comprise 4 repeating units of GGGGS (SEQ ID No. 52), as for the CD8αα homodimer described by Devine et al 1999.

The amino acid sequence for the CD8P Ig-like V-type domain is shown below as SEQ ID No. 54.

(CD8β Ig-like V-type domain)
SEQ ID No. 54
LQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIV
GSPELTFGKGTQL

The engineered polypeptide may comprise a CD8αβ heterodimer in which the CD8α and CD8β domains are in either order in the construct, i.e. CD8αβ or CD8βα.

The engineered polypeptide may comprise a short flexible linker between the CD8α monomer, the CD8αα homodimer or the CD8αβ heterodimer and the stalk and/or transmembrane domain to introduce a chain-break. A chain break separate two distinct domains but allows orientation in different angles. Such sequences include the sequence SDP, and the sequence SGGGSDP (SEQ ID NO: 48).

The linker may comprise a serine-glycine linker, such as SGGGGS (SEQ ID NO: 49).

The engineered polypeptide may comprise a transmembrane domain, as defined above. For example, the engineered polypeptide may comprise the transmembrane domains of CD8-alpha or CD28.

The engineered polypeptide comprises an intracellular signalling domain, as defined above. The engineered polypeptide may, for example, comprise the CD3ζ endodomain.

The engineered polypeptide may have the general structure:

CD4 D1 domain-linker-transmembrane domain-intracellular signalling domain;
CD8α Ig-like V-type domain-linker-transmembrane domain-intracellular signalling domain;
CD8αα homodimer-linker-transmembrane domain-intracellular signalling domain; or
CD8αβ homodimer-linker-transmembrane domain-intracellular signalling domain

### Bi-Specific Polypeptides

In a further embodiment of the present invention, the polypeptide capable of co-localizing the MHC class I polypeptide or an MHC class II polypeptide with an intracellular signalling domain may be a bispecific polypeptide which comprises:

(a) a first binding domain which is binds to an MHC class I polypeptide or an MHC class II polypeptide
(b) a second binding domain which is capable of binding to a polypeptide comprising an intracellular signalling domain or a component of the CD3 complex.

The bispecific polypeptide may be membrane-tethered.

When expressed by the cell or on the cell surface, the present bispecific molecule co-localises MHC class I or II and the TCR, and facilitates TCR signalling in a cell of the invention following binding of a TCR on a different T cell to the peptide/MHC complex bound by the bispecific molecule.

Bispecific molecules have been developed in a number of different formats. One of the most common is a fusion consisting of two single-chain variable fragments (scFvs) of different antibodies.

The first and/or second binding domains of the bispecific molecule may be antibody or immunoglobulin based binding domains.

As used herein, “antibody” means a polypeptide having an antigen binding site which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′₂, Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human.

Alternatively, the first and/or second binding domains of the present bispecific molecule may comprise domains which are not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The first binding domain of the present bispecific molecule is capable of binding to a MCH class I or MHC class II polypeptide.

As mentioned above several antibodies have been described which specifically bind MHC class I or MHC class II.

For example, WO05/023299, which is incorporated by reference, describes antibodies which bind MHC class II antigens, in particular antibodies against the HLA-DR alpha chain. Table 1 of that document contains the sequence characteristics of clones MS-GPC-1 (scFv-17), MS-GPC-6 (scFv-8A), MS-GPC-8 (scFv-B8) and MS-GPC-10 (scFv-E6) and FIG. 15 gives the VH and VL sequences for MS-GPC-1; MS-GPC-6; MS-GPC-8; MS-GPC-10; MS-GPC-8-6; MS-GPC-8-10; MS-GPC-8-17; MS-GPC-8-27; MS-GPC-8-6-13; MS-GPC-8-10-57; MS-GPC-8-27-41; MS-GPC-8-1; MS-GPC-8-9; MS-GPC-8-18; MS-GPC-8-6-2; MS-GPC-8-6-19; MS-GPC-8-6-27; MS-GPC-8-6-45; MS-GPC-8-6-47; MS-GPC-8-27-7; and MS-GPC-8-27-10.

The bispecific polypeptide may comprise an MHC class II binding domain comprising one of these pairs of VH and VL sequences. In particular, bispecific polypeptide may comprise an MHC class II binding domain based on the binder MS-GPC-8.

Andris et al (1995 Mol Immunol 32:14-15) describe six antibodies specific for human HLA class I and class II antigens including an antibody against HLA-DQ beta chain having the antibody clone name anti-HLAII/DQB1-MP1.

Watkins et al (2000 Tissue Antigens 55: 219-28) describe the isolation and characterisation of human monoclonal HLA-A2 antibodies. The antibody clones include: anti-HLA-A2/A28-3PF12, anti-HLA-A2/A28-3PC4 and anti-HLA-A2/A28-3PB2.

The bispecific polypeptide of the present invention may comprise an MHC class I or MHC class II binding domain derived from any of these antibodies.

The second domain of the present bispecific molecule is capable of binding to a polypeptide comprising an intracellular signalling domain or a component of the CD3 complex. In particular, the second domain may be capable of binding CD3 on the T-cell surface. In this respect, the second domain may comprise a CD3 or TCR-specific antibody or part thereof.

The second domain may comprise the complementarity determining regions (CDRs) from the scFv sequence shown as SEQ ID NO: 55.

The second domain may comprise a scFv sequence, such as the one shown as SEQ ID NO: 55. The second domain may comprise a variant of such a sequence which has at least 80% sequence identity and binds CD3.

The second domain may comprise an antibody or part thereof which specifically binds CD3, such as OKT3, WT32, anti-leu-4, UCHT-1, SPV-3TA, TR66, SPV-T3B or affinity tuned variants thereof.

The second domain of the bispecific molecule of the invention may comprise all or part of the monoclonal antibody OKT3, which was the first monoclonal antibody approved by the FDA. OKT3 is available from ATCC CRL 8001. The antibody sequences are published in U.S. Pat. No. 7,381,803.

The second domain may comprise one or more CDRs from OKT3. The second binding domain may comprise CDR3 from the heavy-chain of OKT3 and/or CDR3 from the light chain of OKT3. The second binding domain may comprise all 6 CDRs from OKT3, as shown below.

Heavy Chain
CDR1:
(SEQ ID NO: 56)
KASGYTFTRYTMH
CDR2:
(SEQ ID NO: 57)
INPSRGYTNYNQKFKD
CDR3:
(SEQ ID NO: 58)
YYDDHYCLDY
Light Chain
CDR1:
(SEQ ID NO: 59)
SASSSVSYMN
CDR2:
(SEQ ID NO: 60)
RWIYDTSKLAS
CDR3:
(SEQ ID NO: 61)
QQWSSNPFT

The second binding domain may comprise a scFv which comprises the CDR sequences from OKT3. The second binding domain may comprise the scFv sequence shown below as SEQ ID NO: 55 or 62 or a variant thereof having at least 80% sequence identity, which retains the capacity to bind CD3.

SEQ ID NO: 55
QVQLQQSGAELARPGASVKMSCKASGYTFTRYTMHVKQRPGQGLEWIGY
INPSRGYTNYNQKFKDKATLTTDKSSSTAYMQLSSLTSEDSAVYYCARY
YDDHYCLDYWGQGTTLTVSSSGGGGSGGGGSGGGGSQIVLTQSPAIMSA
SPGEKVTMTCSASSSVSYMNWYQQKSGTSPKRWIYDTSKLASGVPAHFR
GSGSGTSYSLTISGMEAEDAATYYCQQWSSNPFTFGSGTKLEINR
SEQ ID NO: 62
QIVLTQSPAIMSASPGEKVTMTCSASSSVSYMNWYQQKSGTSPKRWIYD
TSKLASGVPAHFRGSGSGTSYSLTISGMEAEDAATYYCQQWSSNPFTFG
SGTKLEINRSSSGGGGSGGGGSGGGGSQVQLQQSGAELARPGASVKMSC
KASGYTFTRYTMHWVKQRPGQGLEWIGYINPSRGYTNYNQKFKDKATLT
TDKSSSTAYMQLSSLTSEDSAVYYCARYYDDHYCLDYWGQGTTLTVSS

SEQ ID NO: 55 and 62 provide alternative architectures of an scFV suitable for use in the present invention. SEQ ID NO: 55 is provided as a VL-VH arrangement. SEQ ID NO: 55 is provided as a VH-VL arrangement.

A variant sequence from SEQ ID NO: 55 or 62 may have at least 80, 85, 90, 95, 98 or 99% sequence identity and have equivalent or improved CD3 binding capabilities as the sequence shown as SEQ ID NO: 55 or 62.

The bispecific molecule of the present invention may comprise a spacer sequence to connect the first domain with the second domain and spatially separate the two domains.

For example, the first and second binding domains may be connected via a short five residue peptide linker (GGGGS).

The spacer sequence may, for example, comprise an IgG1 hinge or a CD8 stalk. The linker may alternatively comprise an alternative linker sequence which has similar length and/or domain spacing properties as an IgG1 hinge or a CD8 stalk.

The spacer may be a short spacer, for example a spacer which comprises less than 100, less than 80, less than 60 or less than 45 amino acids. The spacer may be or comprise an IgG1 hinge or a CD8 stalk or a modified version thereof.

Examples of amino acid sequences for these linkers are given below:

SEQ ID NO: 63 (IgG1 hinge):
AEPKSPDKTHTCPPCPKDPKSGGGGS
SEQ ID NO: 64 (CD8 stalk):
TTTPAPRPPTPAPTIASQPLSLRPEACRPAAGGAVHTRGLDFACD

The CD8 stalk has a sequence such that it may induce the formation of homodimers. If this is not desired, one or more cysteine residues may be substituted or removed from the CD8 stalk sequence. The bispecific molecule of the invention may include a spacer which comprises or consists of the sequence shown as SEQ ID NO: 64 or a variant thereof having at least 80, 85, 90, 95, 98 or 99% sequence identity, provided that the variant sequence is a molecule which causes approximately equivalent spacing of the first and second domains and/or that the variant sequence causes homodimerisation of the bispecific molecule.

The bispecific molecule of the invention may have the general formula:

First domain−spacer−second domain.

The spacer may also comprise one or more linker motifs to introduce a chain-break. A chain break separate two distinct domains but allows orientation in different angles. Such sequences include the sequence SDP, and the sequence SGGGSDP (SEQ ID NO: 48).

The linker may comprise a serine-glycine linker, such as SGGGGS (SEQ ID NO: 49).

The spacer may cause the bispecific molecule to form a homodimer, for example due to the presence of one or more cysteine residues in the spacer, which can for a di-sulphide bond with another molecule comprising the same spacer.

The bispecific molecule may be membrane-tethered. In other words, the bispecific molecule may comprise a transmembrane domain such that it is localised to the cell membrane following expression in the cell of the present invention.

By way of example, the transmembrane domain may a transmembrane domain as described herein. For example, the transmembrane domain may comprise a hydrophobic alpha helix. The transmembrane domain may be derived from CD8alpha or CD28.

The bispecific molecule of the invention may have the general formula:

First domain−spacer−second domain−transmembrane domain; or

Transmembrane domain−first domain−spacer−second domain.

Transgenic T-Cell Receptor The engineered immune cell of the present invention may express a transgenic T-cell receptor (TCR).

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vector. For example the genes for engineered TCRs may be reintroduced into autologous T cells and transferred back into patients for T cell adoptive therapies. Such ‘heterologous’ TCRs may also be referred to herein as ‘transgenic TCRs’.

### Effector Immune Cells and Cell Surface Receptor/Receptor Complexes

The effector immune cell of the present invention may be a cytolytic immune cell such as a T-cell, a natural killer (NK) cell or a cytokine induced killer cell.

The T cell may be an alpha-beta T cell or a gamma-delta T cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukopheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

The cell surface receptor or receptor complex binds an antigen recognition receptor of a target immune cell may be an MHC class I receptor or complex; an MHC class II receptor or complex; or a TCR or TCR/CD3 complex.

### Target Immune Cells and Antigen Recognition Receptors

The target immune cell of the present invention may be a cytolytic immune cell such as a T-cell, a natural killer (NK) cell or a cytokine induced killer cell.

The target immune cell may be present in a population of immune cells in vitro, ex vivo, or in vivo. The target immune cell may, for example, be in a patient or in a transplant, prior to administration to a patient.

The target immune cells may specifically recognise an autoantigen or an alloantigen.

The antigen recognition receptor of the target immune cell may be a T-cell receptor, such as an αβ-TCR or γδ-TCR which are described in more detail above.

Alternatively the antigen recognition receptor may be an NK cell activating receptor. There are two different kinds of surface receptors which are responsible for triggering NK-mediated natural cytotoxicity: the NK KARs (Killer Activation Receptors) and the NK KIRs (Killer Inhibitory Receptors) which produce opposite signals. It is the balance between these competing signals that determines whether or not the cytotoxic activity of the NK cell should be triggered.

KARs typically have noncovalently linked subunits that contain immunoreceptor tyrosine-based activation motifs (ITAMs) in their cytoplasmic tails such as CD3ζ, the γc chain, or one of two adaptor proteins DAP10 and DAP12. In an analogous fashion to the TCR on a T-cell, the ITAMs associated with KARs are involved in the facilitation of signal transduction in NK cells. When the binding of an activation ligand to an KAR complex occurs, the tyrosine residues in the ITAMs in the associated chain are phosphorylated by kinases, and a signal that promotes natural cytotoxicity is conveyed to the interior of the NK cell.

### Engineering to Resist Target Immune Cell “Fight Back”

The effector immune cell of the present invention is engineered such that, when the cell surface receptor or receptor complex of the effector immune cell specifically binds an antigen recognition receptor of a target immune cell, the effector immune cell wins the battle between the two immune cells, such that the target immune cell is killed by the effector immune cell, rather than the effector immune cell being killed by the target immune cell.

There are various ways in which the effector immune cell can be engineered to have a selective advantage over the target immune cell at the time and place where the two cells encounter each other.

For example:

1) the effector immune cell may be engineered such that it is resistant to one or more immunosuppressive drugs
2) the effector immune cell may be engineered such that it is capable of transmitting one or more inhibitory immune signals

### Resistance to Immunosuppression

The effector immune cell may be engineered such that it is resistant to one or more immunosuppressive drugs. This means that in the presence of the immunosuppressive drug, the target immune cell will be suppressed and the effector cell will be resistant to suppression, giving the effector immune cell a selective advantage.

The immunosuppressive drug may be administered to population of immune cells in vivo or in vitro. For example, the immunosuppressive drug may be administered to a patient prior to or at the same time as administration of a composition comprising the effector immune cells. Alternatively the immunosuppressive drug may be administered to a transplant prior to or at the same time as administration of a composition comprising the effector immune cells to the transplant and before the transplant is introduced into a patient.

Immunosuppressive drugs, also known as immunosuppressive agents, immunosuppressants and antirejection medications are drugs that inhibit or prevent activity of the immune system. Immunosuppressive drugs are commonly used in immunosuppressive therapy, for example to:

(i) prevent the rejection of transplanted organs and tissues (e.g., bone marrow, heart, kidney, liver) and cells (e.g. during hematopoietic stem cell transplantation and allogeneic immunotherapy approaches);
(ii) treat autoimmune diseases or diseases that are most likely of autoimmune origin (e.g., rheumatoid arthritis, multiple sclerosis, myasthenia gravis, psoriasis, vitiligo, granulomatosis with polyangiitis, systemic lupus erythematosus, systemic sclerosis/scleroderma, sarcoidosis, focal segmental glomerulosclerosis, Crohn's disease, Behcet's Disease, pemphigus, and ulcerative colitis); and
(iii) treat some other non-autoimmune inflammatory diseases (e.g., long term allergic asthma control), ankylosing spondylitis.

A large number of immunosuppressive drugs are known and routinely used during transplants and immunotherapy approaches. The immunosuppressive drug may be, for example, a small molecule or an antibody or other biologic. The immunosuppressive drug may be a glucocorticoid, cytostatic, a polyclonal or monoclonal antibody or a drug which acts on immunophilins. These are described in more detail below.

### Glucocorticoids

Glucocorticoids are a class of corticosteroids, which are a class of steroid hormones. Glucocorticoids are corticosteroids that bind to the glucocorticoid receptor. Examples include: cortisol (hydrocortisone), cortisone, prednisone, prednisolone, methylprednisolone, dexamethasone, betamethasone, triamcinolone, fludrocortisone acetate and deoxycorticosterone acetate.

In pharmacologic (i.e. supraphysiologic) doses, glucocorticoids are used to suppress various allergic, inflammatory, and autoimmune disorders. They are also administered as post-transplantory immunosuppressants to prevent the acute transplant rejection and graft-versus-host disease.

Glucocorticoids suppress the cell-mediated immunity. They act by inhibiting genes that code for the cytokines Interleukin 1 (IL-1), IL-2, IL-3, IL-4, IL-5, IL-6, IL-8, and TNF-alpha, the most important of which is IL-2. A lower level of cytokine production reduces T cell proliferation. Glucocorticoids also suppress the humoral immunity, causing B cells to express smaller amounts of IL-2 and IL-2 receptors. This diminishes both B cell clone expansion and antibody synthesis.

Glucocorticoids influence all types of inflammatory events, no matter their cause. They induce the lipocortin-1 (annexin-1) synthesis, which then binds to cell membranes preventing the phospholipase A2 from coming into contact with its substrate arachidonic acid. This leads to diminished eicosanoid production. The cyclooxygenase (both COX-1 and COX-2) expression is also suppressed, potentiating the effect.

Glucocorticoids also stimulate the lipocortin-1 escaping to the extracellular space, where it binds to the leukocyte membrane receptors and inhibits various inflammatory events: epithelial adhesion, emigration, chemotaxis, phagocytosis, respiratory burst, and the release of various inflammatory mediators (lysosomal enzymes, cytokines, tissue plasminogen activator, chemokines, etc.) from neutrophils, macrophages, and mastocytes.

### Cytostatics

Cytostatics inhibit cell division. In immunotherapy, they are used in smaller doses than in the treatment of malignant diseases. They affect the proliferation of both T cells and B cells. Due to their highest effectiveness, purine analogs are most frequently administered. Cytostatics include alkylating agents, antimetabolites, methotrexate, azathioprine and mercaptopurine, and cytotoxic antibiotics.

The alkylating agents used in immunotherapy are nitrogen mustards (cyclophosphamide), nitrosoureas, platinum compounds, and others. Cyclophosphamide (Baxter's Cytoxan) is probably the most potent immunosuppressive compound. In small doses, it is very efficient in the therapy of systemic lupus erythematosus, autoimmune hemolytic anemias, granulomatosis with polyangiitis, and other immune diseases. High doses cause pancytopenia and hemorrhagic cystitis.

Antimetabolites interfere with the synthesis of nucleic acids. These include: folic acid analogues, such as methotrexate; purine analogues, such as azathioprine and mercaptopurine; pyrimidine analogues, such as fluorouracil; and protein synthesis inhibitors.

Methotrexate is a folic acid analogue. It binds dihydrofolate reductase and prevents synthesis of tetrahydrofolate. It is used in the treatment of autoimmune diseases (for example rheumatoid arthritis or Behcet's Disease) and in transplantations.

Azathioprine (Prometheus' Imuran), is the main immunosuppressive cytotoxic substance. It is extensively used to control transplant rejection reactions. It is nonenzymatically cleaved to mercaptopurine, that acts as a purine analogue and an inhibitor of DNA synthesis. Mercaptopurine itself can also be administered directly.

By preventing the clonal expansion of lymphocytes in the induction phase of the immune response, it affects both the cell and the humoral immunity. It is also efficient in the treatment of autoimmune diseases.

Among the cytotoxic antibiotics, dactinomycin is the most important. It is used in kidney transplantations. Other cytotoxic antibiotics are anthracyclines, mitomycin C, bleomycin, mithramycin.

### Antibodies

Antibodies are sometimes used as a quick and potent immunosuppressive therapy to prevent the acute rejection reactions as well as a targeted treatment of lymphoproliferative or autoimmune disorders (e.g., anti-CD20 monoclonals). They may be polyclonal or monoclonal.

Heterologous polyclonal antibodies are obtained from the serum of animals (e.g., rabbit, horse), and injected with the patient's thymocytes or lymphocytes. The antilymphocyte (ALG) and antithymocyte antigens (ATG) are being used. They are part of the steroid-resistant acute rejection reaction and grave aplastic anemia treatment. However, they are added primarily to other immunosuppressives to diminish their dosage and toxicity. They also allow transition to cyclosporin therapy.

Polyclonal antibodies inhibit T lymphocytes and cause their lysis, which is both complement-mediated cytolysis and cell-mediated opsonization followed by removal of reticuloendothelial cells from the circulation in the spleen and liver. In this way, polyclonal antibodies inhibit cell-mediated immune reactions, including graft rejection, delayed hypersensitivity (i.e., tuberculin skin reaction), and the graft-versus-host disease (GVHD), but influence thymus-dependent antibody production.

Two preparations available to the market are: Atgam, obtained from horse serum, and Thymoglobuline, obtained from rabbit serum. Polyclonal antibodies affect all lymphocytes and cause general immunosuppression, possibly leading to post-transplant lymphoproliferative disorders (PTLD) or serious infections, especially by cytomegalovirus. To reduce these risks, treatment is provided in a hospital, where adequate isolation from infection is available.

Monoclonal antibodies cause fewer side-effects. Especially significant are the IL-2 receptor- (CD25-) and CD3-directed antibodies. They are used to prevent the rejection of transplanted organs, but also to track changes in the lymphocyte subpopulations. It is reasonable to expect similar new drugs in the future.

Muromonab-CD3 is a murine anti-CD3 monoclonal antibody of the IgG2a type that prevents T-cell activation and proliferation by binding the T-cell receptor complex present on all differentiated T cells. As such it is one of the most potent immunosuppressive substances and is administered to control the steroid- and/or polyclonal antibodies-resistant acute rejection episodes. As it acts more specifically than polyclonal antibodies it is also used prophylactically in transplantations.

Interleukin-2 is an important immune system regulator necessary for the clone expansion and survival of activated lymphocytes T. Its effects are mediated by the trimer cell surface receptor IL-2a, consisting of the α, β, and γ chains. The IL-2a (CD25, T-cell activation antigen, TAC) is expressed only by the already-activated T lymphocytes. Therefore, it is of special significance to the selective immunosuppressive treatment, and research has been focused on the development of effective and safe anti-IL-2 antibodies. Basiliximab (Simulect) and daclizumab (Zenapax) are chimeric mouse/human anti-Tac antibodies. These drugs act by binding the IL-2a receptor's α chain, preventing the IL-2 induced clonal expansion of activated lymphocytes and shortening their survival. They are used, for example in the prophylaxis of the acute organ rejection after bilateral kidney transplantation.

### Calcineurin Inhibitors and Other Drugs

Tacrolimus and cyclosporin are a calcineurin inhibitor (CNI). Calcineurin has been in use since 1983 and is one of the most widely used immunosuppressive drugs. It is a cyclic fungal peptide, composed of 11 amino acids.

Cyclosporin is thought to bind to the cytosolic protein cyclophilin (an immunophilin) of immunocompetent lymphocytes, especially T-lymphocytes. This complex of cyclosporin and cyclophilin inhibits the phosphatase calcineurin, which under normal circumstances induces the transcription of interleukin-2. The drug also inhibits lymphokine production and interleukin release, leading to a reduced function of effector T-cells.

Tacrolimus is a product of the bacterium Streptomyces tsukubaensis. It is a macrolide lactone and acts by inhibiting calcineurin.

The drug is used primarily in liver and kidney transplantations, although in some clinics it is used in heart, lung, and heart/lung transplantations. It binds to the immunophilin FKBP1A, followed by the binding of the complex to calcineurin and the inhibition of its phosphatase activity. In this way, it prevents the cell from transitioning from the G0 into G1 phase of the cell cycle. Tacrolimus is more potent than cyclosporin and has less pronounced side-effects.

Sirolimus (rapamycin) is a macrolide lactone, produced by the actinomycete bacterium Streptomyces hygroscopicus. It is used to prevent rejection reactions.

Although it is a structural analogue of tacrolimus, it acts somewhat differently and has different side-effects.

Contrary to cyclosporin and tacrolimus which affect the first phase of T lymphocyte activation, sirolimus affects the second phase, namely signal transduction and lymphocyte clonal proliferation. It binds to FKBP1A like tacrolimus, however the complex does not inhibit calcineurin but another protein, mTOR. Therefore, sirolimus acts synergistically with cyclosporin and, in combination with other immunosuppressants, has few side effects. Also, it indirectly inhibits several T lymphocyte-specific kinases and phosphatases, hence preventing their transition from G1 to S phase of the cell cycle. In a similar manner, Sirolimus prevents B cell differentiation into plasma cells, reducing production of IgM, IgG, and IgA antibodies. It is also active against tumors that are PI3K/AKT/mTOR-dependent.

Everolimus is an analog of sirolimus and also is an mTOR inhibitor.

Other immunosuppressive drugs include interferons, opioids, TNF binding proteins, mycophenolate and small biological agents.

IFN-β suppresses the production of Th1 cytokines and the activation of monocytes. It is used to slow down the progression of multiple sclerosis. IFN-γ is able to trigger lymphocytic apoptosis.

Opioids are substances that act on opioid receptors to produce morphine-like effects. Prolonged use of opioids may cause immunosuppression of both innate and adaptive immunity. Decrease in proliferation as well as immune function has been observed in macrophages, as well as lymphocytes. It is thought that these effects are mediated by opioid receptors expressed on the surface of these immune cells.

A TNF-α (tumor necrosis factor-alpha) binding protein is a monoclonal antibody or a circulating receptor such as infliximab (Remicade), etanercept (Enbrel), or adalimumab (Humira) that binds to TNF-α, preventing it from inducing the synthesis of IL-1 and IL-6 and the adhesion of lymphocyte-activating molecules. They are used in the treatment of rheumatoid arthritis, ankylosing spondylitis, Crohn's disease, and psoriasis.

TNF or the effects of TNF are also suppressed by various natural compounds, including curcumin (an ingredient in turmeric) and catechins (in green tea).

Mycophenolic acid acts as a non-competitive, selective, and reversible inhibitor of Inosine-5′-monophosphate dehydrogenase (IMPDH), which is a key enzyme in the de novo guanosine nucleotide synthesis. In contrast to other human cell types, lymphocytes B and T are very dependent on this process. Mycophenolate mofetil is used in combination with cyclosporin or tacrolimus in transplant patients.

Small biological agents include fingolimod which is a synthetic immunosuppressant. It increases the expression or changes the function of certain adhesion molecules (α4/β7 integrin) in lymphocytes, so they accumulate in the lymphatic tissue (lymphatic nodes) and their number in the circulation is diminished. In this respect, it differs from all other known immunosuppressants.

Myriocin is an atypical amino acid and an antibiotic derived from certain thermophilic fungi. It has been shown to inhibit the proliferation of cytotoxic T-cells.

### Resistance by Mutation

The effector cell of the present invention may comprise one or more mutations which increases its resistance to one or more immune suppressive drugs. For example, effector cell may comprise one or more mutations which renders the cell resistant to tacrolimus and/or cyclosporin.

The effector cell may comprise a nucleic acid sequence encoding calcineurin (CN) with one or more mutations. Calcineurin (CaN) is a calcium and calmodulin dependent serine/threonine protein phosphatase which activates the T cells of the immune system. Calcineurin activates nuclear factor of activated T cell cytoplasmic (NFATc), a transcription factor, by dephosphorylating it. The activated NFATc is then translocated into the nucleus, where it upregulates the expression of interleukin 2 (IL-2), stimulating the T cell response. Calcineurin is the target of a class of drugs called calcineurin inhibitors, which include cyclosporin, voclosporin, pimecrolimus and tacrolimus. Brewin et al (2009; Blood 114: 4792-4803) describe various calcineurin mutants which render cytotoxic T lymphocytes resistant to tacrolimus and/or cyclosporin.

Calcineurin is a heterodimer of a 61-kD calmodulin-binding catalytic subunit, calcineurin A and a 19-kD Ca²⁺-binding regulatory subunit, calcineurin B. There are three isozymes of the catalytic subunit, each encoded by a separate gene (PPP3CA, PPP3CB, and PPP3CC) and two isoforms of the regulatory, also encoded by separate genes (PPP3R1, PPP3R2). The amino acid sequences for all of the polypeptides encoded by these genes are available from Uniprot, with the following accession numbers: PPP3CA: Q08209; PPP3CB: P16298; PPP3CC: P48454; PPP3R1: P63098; and PPP3R2: Q96LZ3.

The amino acid sequence for calcineurin A, alpha isoform is shown below as SEQ ID No. 65

(calcineurin A)
SEQ ID No. 65
MSEPKAIDPKLSTTDRVVKAVPFPPSHRLTAKEVFDNDGKPRVDILKAHL
MKEGRLEESVALRIITEGASILRQEKNLLDIDAPVTVCGDIHGQFFDLMK
LFEVGGSPANTRYLFLGDYVDRGYFSIECVLYLWALKILYPKTLFLLRGN
HECRHLTEYFTFKQECKIKYSERVYDACMDAFDCLPLAALMNQQFLCVHG
GLSPEINTLDDIRKLDRFKEPPAYGPMCDILWSDPLEDFGNEKTQEHFTH
NTVRGCSYFYSYPAVCEFLQHNNLLSILRAHEAQDAGYRMYRKSQTTGFP
SLITIFSAPNYLDVYNNKAAVLKYENNVMNIRQFNCSPHPYWLPNFMDVF
TWSLPFVGEKVTEMLVNVLNICSDDELGSEEDGFDGATAAARKEVIRNKI
RAIGKMARVFSVLREESESVLTLKGLTPTGMLPSGVLSGGKQTLQSATVE
AIEADEAIKGFSPQHKITSFEEAKGLDRINERMPPRRDAMPSDANLNSIN
KALTSETNGTDSNGSNSSNIQ

Mutant calcineurin A may comprise a mutation at one or more of the following positions with reference to SEQ ID No. 65: V314; Y341; M347; T351; W352; S353; L354; F356; and K360.

Mutant calcineurin A may comprise one or more of the following substitution mutations with reference to SEQ ID No. 65:
V314K, V314R or V314F;
Y341F;
M347W, M347R or M347E;
T351E;
W352A, W352C or W352E;
S353H or S353N;
L354A:
F356A; and
K360A or K360F.

Mutant calcineurin A may comprise one or more of the following mutation combinations with reference to SEQ ID No. 65:
L354A and K360A;
L354A and K360F;
T351E and K360F;
W352A and S353H;
T351E and L354A;
W352C and K360F;
W352C; L354A and K360F;
V314K and Y341F; and
V314R and Y341F.

The amino acid sequence for calcineurin B, type 1 is shown below as SEQ ID No. 66

(calcineurin B)
SEQ ID No. 66
MGNEASYPLEMCSHFDADEIKRLGKRFKKLDLDNSGSLSVEEFMSLPEL
QQNPLVQRVIDIFDTDGNGEVDFKEFIEGVSQFSVKGDKEQKLRFAFRI
YDMDKDGYISNGELFQVLKMMVGNNLKDTQLQQIVDKTIINADKDGDGR
ISFEEFCAVVGGLDIHKKMVVDV

Mutant calcineurin B may comprise a mutation at one or more of the following positions with reference to SEQ ID No. 66: Q51; L116; M119; V120; G121; N122; N123; L124; K125; and K165.

Mutant calcineurin B may comprise one or more of the following substitution and optionally insertion mutations with reference to SEQ ID No. 66:
Q51S;
L116R or L116Y;
M119A, M119W or M119-F-Ins;
V120L, V120S, V120D or V120F;
G121-LF-Ins;
N122A, N122H, N122F or N122S;
N123H, N123R, N123F, N123K or N123W;
L124T;
K125A, K125E, K125W, K125-LA-Ins, K125-VQ-Ins or K125-IE-Ins; and
K165Q.

Mutant calcineurin B may comprise one or more of the following mutation combinations with reference to SEQ ID No. 66:
V120S and L124T;
V120D and L124T;
N123W and K125-LA-Ins;
L124T and K125-LA-Ins;
V120D and K125-LA-Ins; and
M119-F-Ins and G121-LF-Ins.

In particular, mutant calcineurin B may comprise the following mutation combination with reference to SEQ ID No. 66: L124T and K125-LA-Ins. This is the module known as “CnB30” described in the Examples section. The CnB30 has the amino acid shown as SEQ ID No. 131.

SEQ ID No. 131
(CnB30)
MGNEASYPLEMCSHFDADEIKRLGKRFKKLDLDNSGSLSVEEFMSLPELQ
QNPLVQRVIDIFDTDGNGEVDFKEFIEGVSQFSVKGDKEQKLRFAFRIYD
MDKDGYISNGELFQVLKMMVGNNTKLADTQLQQIVDKTIINADKDGDGRI
SFEEFCAVVGGLDIHKKMVVDV

In the study described in Brewin et al 2009 (as above), the following CNa mutants showed resistance to FK506:
L354A and K360F;
W352A;
W352C;
T351E and L354A;
M347W; and
M347E.

The following CNa mutants showed resistance to cyclosporin A:
V314K;
V314R;
Y341F;
V314K and Y341F; and
V314R and Y341F.

The following CNb mutants showed resistance to FK506:
N123W;
K125-VQ-Ins;
K125-IE-Ins;
K-125-LA-Ins; and
L124T and K-125-LA-Ins.

The following CNb mutants showed resistance to cyclosporin A:
K125-VQ-Ins;
K125-IE-Ins;
K-125-LA-Ins;
V120S and L124T; and
L124T and K-125-LA-Ins.

In particular, it is reported in Brewin et al 2009 (as above) that:
the combination mutation T351E and L354A in CNa confers resistance to CsA but not FK506;
the combination mutation V314R and Y341F in CNa confers resistance to FK506 but not CsA; and
the combination mutation L124T and K-125-LA-Ins in CNb renders CTLs resistant to both calcineurin inhibitors.

The effector immune cell of the present invention may express a variant calcineurin A comprising one or more mutations in the CNa amino acid sequence and/or a variant calcineurin B comprising one or more mutations in the CNb amino acid sequence, which increases resistance of the effector immune cell to one or more calcineurin inhibitors.

In particular, the effector immune cell may express a variant calcineurin A and/or a variant calcineurin B as listed above which confers resistance to cyclosporin A and/or tacrolimus (FK506).

### Dominant Negative CSK

The effector immune cell may be engineered to express a dominant negative C-terminal Src kinase (dnCSK). It has previously been shown that the function of CAR-expressing cells, such as CAR-T cells, can be enhanced by co-expression of a dnCSK (United Kingdom patent application No. 1919017.2). Expression of dominant negative CSK in a CAR-T cell appears to increase the sensitivity of the CAR-T cell, improving cytotoxicity and cytokine release especially in response to low-density target antigens.

The present inventors have now found that expression of dnCSK also confers on the cell general resistance to immunosuppression. The expression of dnCSK provides a “blanket” resistance to immunosuppression, making the cell less sensitive to immunosuppressive drugs in general.

C-terminal Src kinase (CSK), also known as Tyrosine-protein kinase, is an enzyme which phosphorylates tyrosine residues located in the C-terminal end of Src-family kinases (SFKs) including SRC, HCK, FYN, LCK, LYN and YES1, thus suppressing their activity.

Src Family Kinases (SFKs), such as Lck, are made up of a N-terminal myristoyl group, that permits membrane localisation, attached to an SH4 domain, an SH3 domain, an SH2 domain and a protein tyrosine kinase domain (SH1 domain).

There is a conserved tyrosine residue in the activation loop and one in the C-terminal tail, phosphorylation of the activation loop tyrosine by trans-autophosphorylation increases SFK activity, whereas phosphorylation of the C-terminal tyrosine by C-terminal Src kinase (CSK) inhibits SFK activity

Csk phosphorylates the negative regulatory C-terminal tyrosine residue Y505 of Lck to maintain Lck in an inactive state. In resting T cells, Csk is targeted to lipid rafts through engagement of its SH2 domain with phosphotyrosine residue pY317 of PAG. PAG is expressed as a tyrosine phosphorylated protein in nonstimulated T-cells. This interaction of Csk and PAG allows activation of Csk and inhibition of Lck.

Upon TCR activation, CD45 is excluded from membrane microdomains and dephosphorylates PAG, leading to Csk detaching from the plasma membrane.

The amino acid sequence of human CSK is available from Uniprot Accession No 41240 and is shown below as SEQ ID No. 67. In this sequence, residues 9-70 correspond to the SH3 domain, residues 82-171 correspond to the SH2 domain; and residues 195-449 correspond to the protein kinase domain.

SEQ ID No. 67
(wtCSK)
MSAIQAAWPSGTECIAKYNFHGTAEQDLPFCKGDVLTIVAVTKDPNWYKA
KNKVGREGIIPANYVQKREGVKAGTKLSLMPWFHGKITREQAERLLYPPE
TGLFLVRESTNYPGDYTLCVSCDGKVEHYRIMYHASKLSIDEEVYFENLM
QLVEHYTSDADGLCTRLIKPKVMEGTVAAQDEFYRSGWALNMKELKLLQT
NIGKGEFGDVMLGDYRGNKVAVKCIKDATAQAFLAEASVMTQLRHSNLVQ
LLGVIVEEKGGLYIVTEYMAKGSLVDYLRSRGRSVLGGDCLLKFSLDVCE
AMEYLEGNNFVHRDLAARNVLVSEDNVAKVSDFGLTKEASSTQDTGKLPV
KWTAPEALREKKFSTKSDVWSFGILLWEIYSFGRVPYPRIPLKDVVPRVE
KGYKMDAPDGCPPAVYEVMKNCWHLDAAMRPSFLQLREQLEHIKTHELHL

The cells of the present invention may express a dominant negative C-terminal Src kinase (dnCSK).

The dominant negative CSK may lack a functional protein kinase domain. The dnCSK may not comprise a kinase domain or it may comprise a partially or completely inactive kinase domain. The kinase domain may be inactivated by, for example, truncation or mutation of one or more amino acids.

The dnCSK may, for example, be:
i) a truncated CSK which is recruited to the cell membrane but lacks a functional kinase domain;
ii) a mutated CSK which lacks the capacity to phosphorylate Y505 of Lck; or iii) a mutated CSK whose catalytic activity is inhibited by an agent (see FIG. 14).

The effector immune cell may express a dnCSK which completely lacks a kinase domain. For example, the dnCSK may comprise the SH2 domain and optionally the SH3 domain, but be truncated to remove the kinase domain.

Alternatively, the effector immune cell may express a dnCSK which comprises a partially truncated kinase domain having part of a phosphatase, for example a portion of the sequence from residues 195-449 of SEQ ID No. 67, provided that the truncated kinase has reduced capacity to phosphorylate the C-terminal tyrosine residue Y505 of Lck compared to wild-type CSK. The truncated kinase may have effectively no residual kinase activity.

The dnCSK may be a truncated CSK which retains the capacity to bind a transmembrane adaptor protein such as PAG, Lime and/or Dok1/2 which recruits wild-type CSK to the cell membrane but lacks a functional kinase domain.

The dnCSK may have the sequence shown as SEQ ID No. 68, which corresponds to the wild-type CSK sequence (SEQ ID No. 67) minus the kinase domain.

SEQ ID No. 68
(CSK_del_kinase)
MSAIQAAWPSGTECIAKYNFHGTAEQDLPFCKGDVLTIVAVTKDPNWYKA
KNKVGREGIIPANYVQKREGVKAGTKLSLMPWFHGKITREQAERLLYPPE
TGLFLVRESTNYPGDYTLCVSCDGKVEHYRIMYHASKLSIDEEVYFENLM
QLVEHYTSDADGLCTRLIKPKVMEGTVAAQDEFYRSGWALNMKE

Alternatively the dnCSK may have the sequence shown as SEQ ID No. 69, which corresponds to the wild-type CSK sequence (SEQ ID No. 67) minus the kinase and SH3 domains.

SEQ ID No. 69
(CSK_del_kinase_SH3)
MSAIQAAWVKAGTKLSLMPWFHGKITREQAERLLYPPETGLFLVRESTNY
PGDYTLCVSCDGKVEHYRIMYHASKLSIDEEVYFENLMQLVEHYTSDADG
LCTRLIKPKVMEGTVAAQDEFYRSGWALNMKE

The effector immune cells of the present invention may express a dnCSK which comprises a kinase domain which is inactivated so that it has reduced or no capacity to phosphorylate proteins such as Lck.

The kinase domain may, for example, comprise one or more amino acid mutations such that it has reduced kinase activity compared to the wild-type sequence.

The mutation may, for example, be an addition, deletion or substitution.

The mutation may comprise the deletion or substitution of one or more lysine residues.

The variant kinase sequence may have a mutation to lysine at position 222 with reference to the sequence shown as SEQ ID No. 67.

The dnCSK of the invention may have the sequence shown as SEQ ID No 70, which corresponds to the full length CSK sequence with a K222R substitution. This mutation is shown in bold and underlined in SEQ ID No. 70. Alternatively, the dnCSK of the invention may have a sequence equivalent to SEQ ID No. 70 in which the SH3 domain has been deleted.

SEQ ID No 70
(CSK(K222R))
MSAIQAAWPSGTECIAKYNFHGTAEQDLPFCKGDVLTIVAVTKDPNWYKA
KNKVGREGIIPANYVQKREGVKAGTKLSLMPWFHGKITREQAERLLYPPE
TGLFLVRESTNYPGDYTLCVSCDGKVEHYRIMYHASKLSIDEEVYFENLM
QLVEHYTSDADGLCTRLIKPKVMEGTVAAQDEFYRSGWALNMKELKLLQT
IGKGEFGDVMLGDYRGNKVAVRCIKNDATAQAFLAEASVMTQLRHSNLVQ
LLGVIVEEKGGLYIVTEYMAKGSLVDYLRSRGRSVLGGDCLLKFSLDVCE
AMEYLEGNNFVHRDLAARNVLVSEDNVAKVSDFGLTKEASSTQDTGKLPV
KWTAPEALREKKFSTKSDVWSFGILLWEIYSFGRVPYPRIPLKDVVPRVE
KGYKMDAPDGCPPAVYEVMKNCWHLDAAMRPSFLQLREQLEHIKTHELHL

The dnCSK may comprise a mutated CSK whose catalytic activity is inhibited by an agent. For example, the dnCSK may have the sequence shown as SEQ ID No. 71, which comprises the mutation T266G compared to the wildtype sequence shown as SEQ ID No. 67 and is known as “CSKas”. The substitution is in bold and underlined in SEQ ID No. 71. Alternatively, the dnCSK of the invention may have a sequence equivalent to SEQ ID No. 71 in which the SH3 domain has been deleted.

SEQ ID No. 71
(CSKas)
MSAIQAAWPSGTECIAKYNFHGTAEQDLPFCKGDVLTIVAVTKDPNWYKA
KNKVGREGIIPANYVQKREGVKAGTKLSLMPWFHGKITREQAERLLYPPE
TGLFLVRESTNYPGDYTLCVSCDGKVEHYRIMYHASKLSIDEEVYFENLM
QLVEHYTSDADGLCTRLIKPKVMEGTVAAQDEFYRSGWALNMKELKLLQT
IGKGEFGDVMLGDYRGNKVAVKCIKNDATAQAFLAEASVMTQLRHSNLVQ
LLGVIVEEKGGLYIVGEYMAKGSLVDYLRSRGRSVLGGDCLLKFSLDVCE
AMEYLEGNNFVHRDLAARNVLVSEDNVAKVSDFGLTKEASSTQDTGKLPV
KWTAPEALREKKFSTKSDVWSFGILLWEIYSFGRVPYPRIPLKDVVPRVE
KGYKMDAPDGCPPAVYEVMKNCWHLDAAMRPSFLQLREQLEHIKTHELHL

The catalytic activity of CSKas is inhibited by 3-iodo-benzyl-PP1. In the presence of this molecule, therefore CSKas acts as a dominant negative version of CSK, competing with the wild-type enzyme for binding to membrane proteins such as PAG, Lime and/or Dok1/2 which recruit wild-type CSK to the cell membrane.

### Transmission of Inhibitory Immune Signals

The effector immune cell of the present invention may express or overexpress an immunoinhibitory molecule or a fusion protein comprising the extracellular domain of an immunoinhibitory molecule.

In vivo, membrane-bound immunoinhibitory receptors such as PD-1, LAG-3, 2B4 or BTLA 1 inhibit T cell activation. During T cell activation (illustrated schematically in FIG. 15a), antigen recognition by the T-cell receptor (TCR) results in phosphorylation of Immunoreceptor tyrosine-based activation motifs (ITAMs) on CD3ζ. Phosphorylated ITAMs are recognized by the ZAP70 SH2 domains, leading to T cell activation. As illustrated schematically in FIG. 15b, inhibitory immune-receptors such as PD1 effectively reverse this process. PD1 has ITIMs in its endodomain which are recognized by the SH2 domains of PTPN6 (SHP-1). When PD1 binds its ligand, PD-L1 or a tumour cell, PTPN6 is recruited to the juxta-membrane region and its phosphatase domain subsequently de-phosphorylates ITAM domains inhibiting immune activation.

The target immune cell will naturally express a variety of such ITIM containing immunoinhibitory receptors, such as PD-1, LAG3, TIM-3, TIGIT, BTLA, VISTA, CEACAM1-R, KIR2DL4, B7-H3 and B7-H4.

By engineering the effector immune cell of the invention to express a ligand for one or more immunoinhibitory receptors or the extracellular domain of such a ligand, when a synapse forms between the two cells the effector immune cell will inhibit T cell activation in the target immune cell. This “one-way” inhibition gives the effector immune cell an advantage over the target immune cell in terms of activation meaning that the effector immune cell will prevail, killing the target immune cell.

The effector immune cell may express or overexpress a ligand for an immunoinhibitory receptor on the target immune cell. The immunoinhibitory receptor expressed by the target cell may, for example, be selected from: PD-1, LAG3, TIM-3, TIGIT, BTLA, VISTA, CEACAM1-R, KIR2DL4, B7-H3 and B7-H4.

The immunoinhibitory molecule, or extracellular domain thereof, expressed by the effector immune cell may be, for example, selected from: PD-L1, PD-L2, HVEM, CD155, VSIG-3, Galectin-9, HLA-G, CEACAM-1, LSECTin, FGL1, B7-H3 and B7-H4.

### PD-L1

Programmed death-ligand 1 (PD-L1), also known as cluster of differentiation 274 (CD274) or B7 homolog 1 (B7-H1), is a 40 kDa type 1 transmembrane protein expressed by cancer cells helping them evade anti-tumour immunity. Engagement of PD-L1 with its receptor PD-1 on T cells delivers a signal that inhibits TCR-mediated activation of IL-2 production and T cell proliferation.

The amino acid sequence of human PD-L1 is available from Uniprot, accession No. Q9NZQ7 and shown below as SEQ ID No. 72.

SEQ ID No. 72
(human PD-L1)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

The signal peptide, extracellular domain and transmembrane domain of PD-L1 are shown below as SEQ ID No. 73, 74 and 75 respectively.

SEQ ID No. 73
(Human PD-L1 signal peptide)
MRIFAVFIFMTYWHLLNA
SEQ ID No. 74
(Human PD-L1 extracellular domain)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVISEHELICQAEGYPKAEVIWI
SSDHQVLSGKTTTTNSKREEKLFNVTSTLRINTTTNEIFYCTFRRLDPEE
NHTAELVIPELPLAHPPNER
SEQ ID No. 75
(Human PD-L1 transmembrane domain)
THLVILGAILLCLGVALTFIF

The effector immune cell of the present invention may comprise the PD-L1 extracellular domain and optionally the PD-L1 signal peptide and/or PD-L1 transmembrane domain.

### PD-L2

Programmed cell death 1 ligand 2 (also known as PD-L2, B7-DC) is an immune checkpoint receptor ligand which plays a role in negative regulation of the adaptive immune response. PD-L2 is one of two known ligands for Programmed cell death protein 1 (PD-1), the other being PD-1.

PD-L2 is primarily expressed on professional antigen presenting cells including dendritic cells (DCs) and macrophages. PD-L2 binding to PD-1 can activate pathways inhibiting TCR/BCR-mediated immune cell activation and PD-L2, PD-1, and PD-1 expressions are important in the immune response to certain cancers.

The amino acid sequence of human PD-L2 is available from Uniprot, accession No. Q9BQ51 and shown below as SEQ ID No. 76.

SEQ ID No. 76
(human PD-L2)
MIFLLLMLSLELQLHQIAALFTVTVPKELYIIEHGSNVTLECNFDTGSHV
NLGAITASLQKVENDTSPHRERATLLEEQLPLGKASFHIPQVQVRDEGQY
QCIIIYGVAWDYKYLTLKVKASYRKINTHILKVPETDEVELTCQATGYPL
AEVSWPNVSVPANTSHSRTPEGLYQVTSVLRLKPPPGRNFSCVFWNTHVR
ELTLASIDLQSQMEPRTHPTWLLHIFIPFCIIAFIFIAIVIALRKQLCQK
LYSSKDTTKR PVTTTKREVNSAI

The signal peptide, extracellular domain and transmembrane domain of PD-L2 are shown below as SEQ ID No. 77, 78 and 79 respectively.

SEQ ID No. 77
(Human PD-L2 signal peptide)
MIFLLLMLSLELQLHQIAA
SEQ ID No. 78
(Human PD-L2 extracellular domain)
LFTVTVPKELYIIEHGSNVTLECNFDTGSHVNLGAITASLQKVENDTSPH
RERATLLEQLPLGKASFHIPQVQVRDEGQYQCIIIYGVAWDYKYLTLKVK
ASYRKINTHILKVPETDEVELTCQATGYPLAEVSWPNVSVPANTSHSRTP
EGLYQVTSVLRLKPPPGRNFSCVFWNTHVRELTLASIDLQSQMEPRTHPT
SEQ ID No. 79
(Human PD-L2 transmembrane domain)
WLLHIFIPFCIIAFIFIATVI

The effector immune cell of the present invention may comprise the PD-L2 extracellular domain and optionally the PD-L2 signal peptide and/or PD-L2 transmembrane domain.

### HVEM

Herpesvirus entry mediator (HVEM), also known as tumour necrosis factor receptor superfamily member 14 (TNFRSF14), is a human cell surface receptor of the TNF-receptor superfamily. The cytoplasmic region of this receptor binds to several TNF receptor associated factor (TRAF) family members, which mediate the signal transduction pathways that activate the immune response. TNFRSF14 has been shown to interact with TRAF2, TNFSF14 and TRAF5.

The amino acid sequence of HVEM is available from Uniprot, accession No. Q92956 and shown below as SEQ ID No. 80.

SEQ ID No. 80
(HVEM full sequence)
MEPPGDWGPPPWRSTPKTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH

The signal peptide, extracellular domain and transmembrane domain of HVEM are shown below as SEQ ID No. 81, 82 and 83 respectively.

SEQ ID No. 81
(HVEM signal peptide)
MEPPGDWGPPPWRSTPKTDVLRLVLYLTFLGAPCYAPA
SEQ ID No. 82
(HVEM extracellular domain)
LPSCKEDEYPVGSECCPKCSPGYRVKEACGELTGTVCEPCPPGTYIAHLN
GLSKCLQCQMCDPAMGLRASRNCSRTENAVCGCSPGHFCIVQDGDHCAAC
RAYATSSPGQRVQKGGTESQDTLCQNCPPGTFSPNGTLEECQHQTKCSWL
VTKAGAGTSSSHWW
SEQ ID No. 83
(HVEM transmembrane domain)
WWFLSGSLVIVIVCSTVGLI

The effector immune cell of the present invention may comprise the HVEM extracellular domain and optionally the HVEM signal peptide and/or HVEM transmembrane domain.

### CD155

CD155 (cluster of differentiation 155) also known as the poliovirus receptors is a Type I transmembrane glycoprotein in the immunoglobulin superfamily. CD155 is involved in intestinal humoral immune responses and positive selection of select MHC-independent T cells in the thymus.

The amino acid sequence of CD155 is available from Uniprot, accession No. P15151 and shown below as SEQ ID No. 84.

SEQ ID No. 84
(CD155 full sequence)
MARAMAAAWPLLLVALLVLSWPPPGTGDVVVQAPTQVPGFLGDSVTLPCY
LQVPNMEVTHVSQLTWARHGESGSMAVFHQTQGPSYSESKRLEFVAARLG
AELRNASLRMFGLRVEDEGNYTCLFVTFPQGSRSVDIWLRVLAKPQNTAE
VQKVQLTGEPVPMARCVSTGGRPPAQITWHSDLGGMPNTSQVPGFLSGTV
TVTSLWILVPSSQVDGKNVTCKVEHESFEKPQLLTVNLTVYYPPEVSISG
YDNNWYLGQNEATLTCDARSNPEPTGYNWSTTMGPLPPFAVAQGAQLLIR
PVDKPINTTLICNVTNALGARQAELTVQVKEGPPSEHSGISRNAIIFLVL
GILVFLILLGIGIYFYWSKCSREVLWHCHLCPSSTEHASASANGHVSYSA
VSRENSSSQDPQTEGTR

The signal peptide, extracellular domain and transmembrane domain of CD155 are shown below as SEQ ID No. 85, 86 and 87 respectively.

SEQ ID No. 85
(CD155 signal peptide)
MARAMAAAWPLLLVALLVLS
SEQ ID No. 86
(CD155 extracellular domain)
WPPPGTGDVVVQAPTQVPGFLGDSVTLPCYLQVPNMEVTHVSQLTWARHG
ESGSMAVFHQTQGPSYSESKRLEFVAARLGAELRNASLRMFGLRVEDEGN
YTCLFVTFPQGSRSVDIWLRVLAKPQNTAEVQKVQLTGEPVPMARCVSTG
GRPPAQITWHSDLGGMPNTSQVPGFLSGTVTVTSLWILVPSSQVDGKNVT
CKVEHESFEKPQLLTVNLTVYYPPEVSISGYDNNWYLGQNEATLTCDARS
NPEPTGYNWSTTMGPLPPFAVAQGAQLLIRPVDKPINTTLICNVTNALGA
RQAELTVQVKEGPPSEHSGISRN
SEQ ID No. 87
(CD155 transmembrane domain)
AIIFLVLGILVFLILLGIGIYFYW

The effector immune cell of the present invention may comprise the CD155 extracellular domain and optionally the CD155 signal peptide and/or CD155 transmembrane domain.

### VSIG-3

VSIG-3, also known as IGSF11, is a ligand of B7 family member VISTA. VSIG-3 inhibits human T-cell proliferation in the presence of T-cell receptor signalling and significantly reduces cytokine and chemokine production by human T cells including IFN-γ, IL-2, IL-17, CCL5/Rantes, CCL3/MIP-1a, and CXCL11/I-TAC.

The amino acid sequence of VSIG-3 is available from Uniprot, accession No. Q5DX21 and shown below as SEQ ID No. 88.

SEQ ID No. 88
(VSIG-3 full sequence)
MTSQRSPLAPLLLLSLHGVAASLEVSESPGSIQVARGQPAVLPCTFTTSA
ALINLNVIWMVTPLSNANQPEQVILYQGGQMFDGAPRFHGRVGFTGTMPA
TNVSIFINNTQLSDTGTYQCLVNNLPDIGGRNIGVTGLTVLVPPSAPHCQ
IQGSQDIGSDVILLCSSEEGIPRPTYLWEKLDNTLKLPPTATQDQVQGTV
TIRNISALSSGLYQCVASNAIGTSTCLLDLQVISPQPRNIGLIAGAIGTG
AVIIIFCIALILGAFFYWRSKNKEEEEEEIPNEIREDDLPPKCSSAKAFH
TEISSSDNNTLTSSNAYNSRYWSNNPKVHRNTESVSHFSDLGQSFSFHSG
NANIPSIYANGTHLVPGQHKTLVVTANRGSSPQVMSRSNGSVSRKPRPPH
THSYTISHATLERIGAVPVMVPAQSRAGSLV

The signal peptide, extracellular domain and transmembrane domain of VSIG-3 are shown below as SEQ ID No. 89, 90 and 91 respectively.

(VSIG-3 signal peptide)
SEQ ID No. 89
MTSQRSPLAPLLLLSLHGVAAS
(VSIG-3 extracellular domain)
SEQ ID No. 90
LEVSESPGSIQVARGQPAVLPCTFTTSAALINLNVIWMVTPLSNANQPE
QVILYQGGQMFDGAPRFHGRVGFTGTMPATNVSIFINNTQLSDTGTYQC
LVNNLPDIGGRNIGVTGLTVLVPPSAPHCQIQGSQDIGSDVILLCSSEE
GIPRPTYLWEKLDNTLKLPPTATQDQVQGTVTIRNISALSSGLYQCVAS
NAIGTSTCLLDLQVISPQPRNIG
(VSIG-3 transmembrane domain)
SEQ ID No. 91
LIAGAIGTGAVIIIFCIALIL

The effector immune cell of the present invention may comprise the VSIG-3 extracellular domain and optionally the VSIG-3 signal peptide and/or VSIG-3 transmembrane domain.

### Galectin-9

Galectin-9 is a ligand for HAVCR2 (TIM-3) and is expressed on various tumour cells. The interaction between galectin-9 and HANCR2 attenuates T-cell expansion and effector function in the tumor microenvironment. Binding to HAVCR2 induces T-helper type 1 lymphocyte (Th1) death. Galectin-9 has N- and C-terminal carbohydrate-binding domains connected by a link peptide.

The amino acid sequence of Galectin-9 is available from Uniprot, accession No. O00182 and shown below as SEQ ID No. 92.

(Galectin-9 full sequence)
SEQ ID No. 92
MAFSGSQAPYLSPAVPFSGTIQGGLQDGLQITVNGTVLSSSGTRFAVNF
QTGFSGNDIAFHFNPRFEDGGYVVCNTRQNGSWGPEERKTHMPFQKGMP
FDLCFLVQSSDFKVMVNGILFVQYFHRVPFHRVDTISVNGSVQLSYISF
QNPRTVPVQPAFSTVPFSQPVCFPPRPRGRRQKPPGVWPANPAPITQTV
IHTVQSAPGQMFSTPAIPPMMYPHPAYPMPFITTILGGLYPSKSILLSG
TVLPSAQRFHINLCSGNHIAFHLNPRFDENAVVRNTQIDNSWGSEERSL
PRKMPFVRGQSFSVWILCEAHCLKVAVDGQHLFEYYHRLRNLPTINRLE
VGGDIQLTHVQT

The signal peptide, Galectin 1 domain and Galectin 2 domain of Galectin-9 are shown below as SEQ ID No. 93, 94 and 95 respectively.

(Galectin-9 signal peptide)
SEQ ID No. 93
MAFSGSQAPYLSPAVP
(Galectin-1 domain)
SEQ ID No. 94
FSGTIQGGLQDGLQITVNGTVLSSSGTRFAVNFQTGFSGNDIAFHFNPR
FEDGGYWCNTRQNGSWGPEERKTHMPFQKGMPFDLCFLVQSSDFKVMVN
GILFVQYFHRVPFHRVDTISVNGSVQLSYISFQ
(Galectin-2 domain)
SEQ ID No. 95
FITTILGGLYPSKSILLSGTVLPSAQRFHINLCSGNHIAFHLNPRFDEN
AVVRNTQIDNSWGSEERSLPRKMPFVRGQSFSVWILCEAHCLKVAVDGQ
HLFEYYHRLRNLPTINRLEVGGDIQLTHVQT

The effector immune cell of the present invention may comprise the full length Galectin-9 sequence, with or without the signal peptide. Alternatively the effector immune cell may just comprise the Galectin 1 domain or the Galectin 2 domain or the HAVCR2-binding domain from Galectin-9, -1 or -2.

The effector immune cell of the present invention may comprise a membrane-tethered version of galectin-9 or a portion thereof. Galectin-9 may be tethered to the membrane using a transmembrane domain and optionally a spacer sequence and/or endodomain. For example, galectin-9 or a portion thereof could be tethered to the membrane using the CD8 stalk spacer, transmembrane domain and truncated endodomain which has been previously described in WO2013/153391 for the sort-suicide gene RQR8

### HLA-G

HLA-G histocompatibility antigen, class I, G, also known as human leukocyte antigen G (HLA-G) belongs to the HLA nonclassical class I heavy chain paralogues. This class I molecule is a heterodimer consisting of a heavy chain and a light chain (beta-2 microglobulin). HLA-G is a ligand for NK cell inhibitory receptor KIR2DL4, and, during pregnancy, expression of this HLA by the trophoblast defends it against NK cell-mediated death.

The amino acid sequence of HLA-G is available from Uniprot, accession No. P17693 and shown below as SEQ ID No. 96.

(HLA-G full sequence)
SEQ ID No. 96
MVVMAPRTLFLLLSGALTLTETWAGSHSMRYFSAAVSRPGRGEPRFIAM
GYVDDTQFVRFDSDSACPRMEPRAPWVEQEGPEYWEEETRNTKAHAQTD
RMNLQTLRGYYNQSEASSHTLQWMIGCDLGSDGRLLRGYEQYAYDGKDY
LALNEDLRSWTAADTAAQISKRKCEAANVAEQRRAYLEGTCVEWLHRYL
ENGKEMLQRADPPKTHVTHHPVFDYEATLRCWALGFYPAEIILTWQRDG
EDQTQDVELVETRPAGDGTFQKWAAVVVPSGEEQRYTCHVQHEGLPEPL
MLRWKQSSLPTIPIMGIVAGLVVLAAVVTGAAVAAVLWRKKSSD

The signal peptide, extracellular domain and transmembrane domain of HLA-G are shown below as SEQ ID No. 97, 98 and 99 respectively.

(HLA-G signal peptide)
SEQ ID No. 97
MVVMAPRTLFLLLSGALTLTETWA
(HLA-G extracellular domain)
SEQ ID No. 98
GSHSMRYFSAAVSRPGRGEPRFIAMGYVDDTQFVRFDSDSACPRMEPRA
PWVEQEGPEYWEEETRNTKAHAQTDRMNLQTLRGYYNQSEASSHTLQWM
IGCDLGSDGRLLRGYEQYAYDGKDYLALNEDLRSWTAADTAAQISKRKC
EAANVAEQRRAYLEGTCVEWLHRYLENGKEMLQRADPPKTHVTHHPVFD
YEATLRCWALGFYPAEIILTWQRDGEDQTQDVELVETRPAGDGTFQKWA
AVVVPSGEEQRYTCHVQHEGLPEPLMLRWKQSSLPTIPI
(HLA-G transmembrane domain)
SEQ ID No. 99
MGIVAGLVVLAAVVTGAAVAAVLW

The effector immune cell of the present invention may comprise the HLA-G extracellular domain and optionally the HLA-G signal peptide and/or HLA-G transmembrane domain.

### CEACAM-1

Carcinoembryonic antigen-related cell adhesion molecule 1 (biliary glycoprotein) (CEACAM1) also known as CD66a (Cluster of Differentiation 66a), is a human glycoprotein, and a member of the carcinoembryonic antigen (CEA) gene family.

CEACAM-1 plays a role as coinhibitory receptor in the immune response of T cells, natural killer (NK) and neutrophils. Upon TCR/CD3 complex stimulation, CEACAM-1 inhibits TCR-mediated cytotoxicity by blocking granule exocytosis by mediating homophilic binding to adjacent cells, allowing interaction with and phosphorylation by LCK and interaction with the TCR/CD3 complex which recruits PTPN6 resulting in dephosphorylation of CD247 and ZAP70. CEACAM-1 also inhibits T cell proliferation and cytokine production through inhibition of the JNK cascade and plays a crucial role in regulating autoimmunity and anti-tumor immunity by inhibiting T cell through its interaction with HAVCR2. Upon natural killer (NK) cells activation, CEACAM-1 inhibits KLRK1-mediated cytolysis of CEACAM1-bearing tumor cells by trans-homophilic interactions with CEACAM1 on the target cell and lead to cis-interaction between CEACAM1 and KLRK1, allowing PTPN6 recruitment and then VAV1 dephosphorylation.

The amino acid sequence of CEACAM-1 is available from Uniprot, accession No. P13688 and shown below as SEQ ID No. 100.

(CEACAM-1 full sequence)
SEQ ID No. 100
MGHLSAPLHRVRVPWQGLLLTASLLTFWNPPTTAQLTTESMPFNVAEGK
EVLLLVHNLPQQLFGYSWYKGERVDGNRQIVGYAIGTQQATPGPANSGR
ETIYPNASLLIQNVTQNDTGFYTLQVIKSDLVNEEATGQFHVYPELPKP
SISSNNSNPVEDKDAVAFTCEPETQDTTYLWWINNQSLPVSPRLQLSNG
NRTLTLLSVTRNDTGPYECEIQNPVSANRSDPVTLNVTYGPDTPTISPS
DTYYRPGANLSLSCYAASNPPAQYSWLINGTFQQSTQELFIPNITVNNS
GSYTCHANNSVTGCNRTTVKTIIVTELSPVVAKPQIKASKTTVTGDKDS
VNLTCSTNDTGISIRWFFKNQSLPSSERMKLSQGNTTLSINPVKREDAG
TYWCEVFNPISKNQSDPIMLNVNYNALPQENGLSPGAIAGIVIGVVALV
ALIAVALACFLHFGKTGRASDQRDLTEHKPSVSNHTQDHSNDPPNKMNE
VTYSTLNFEAQQPTQPTSASPSLTATEIIYSEVKKQ

The signal peptide, extracellular domain and transmembrane domain of CEACAM-1 are shown below as SEQ ID No. 101, 102 and 103 respectively.

(CEACAM-1 signal peptide)
SEQ ID No. 101
MGHLSAPLHRVRVPWQGLLLTASLLTFWNPPTTA
(CEACAM-1 extracellular domain)
SEQ ID No. 102
QLTTESMPFNVAEGKEVLLLVHNLPQQLFGYSWYKGERVDGNRQIVGYA
IGTQQATPGPANSGRETIYPNASLLIQNVTQNDTGFYTLQVIKSDLVNE
EATGQFHVYPELPKPSISSNNSNPVEDKDAVAFTCEPETQDTTYLWWIN
NQSLPVSPRLQLSNGNRTLTLLSVTRNDTGPYECEIQNPVSANRSDPVT
LNVTYGPDTPTISPSDTYYRPGANLSLSCYAASNPPAQYSWLINGTFQQ
STQELFIPNITVNNSGSYTCHANNSVTGCNRTTVKTIIVTELSPVVAKP
QIKASKTTVTGDKDSVNLTCSTNDTGISIRWFFKNQSLPSSERMKLSQG
NTTLSINPVKREDAGTYWCEVFNPISKNQSDPIMLNVNYNALPQENGLS
PG
(CEACAM-1 transmembrane domain)
SEQ ID No. 103
AIAGIVIGVVALVALIAVALACF

The effector immune cell of the present invention may comprise the CEACAM-1 extracellular domain and optionally the CEACAM-1 signal peptide and/or CEACAM-1 transmembrane domain.

### LSECTin

LSECTin, or Liver sinusoidal endothelial cell lectin, is a ligand for LAG-3 and negative regulator of T-cell proliferation and T-cell mediated immunity

The amino acid sequence of LSECTin is available from Uniprot, accession No. Q6UXB4 and shown below as SEQ ID No. 104.

(LSECTin full sequence)
SEQ ID No. 104
MDTTRYSKWGGSSEEVPGGPWGRWVHWSRRPLFLALAVLVTTVLWAVI
LSILLSKASTERAALLDGHDLLRTNASKQTAALGALKEEVGDCHSCCS
GTQAQLQTTRAELGEAQAKLMEQESALRELRERVTQGLAEAGRGREDV
RTELFRALEAVRLQNNSCEPCPTSWLSFEGSCYFFSVPKTTWAAAQDH
CADASAHLVIVGGLDEQGFLTRNTRGRGYWLGLRAVRHLGKVQGYQWV
DGVSLSFSHWNQGEPNDAWGRENCVMMLHTGLWNDAPCDSEKDGWICE
KRHNC

The cytoplasmic domain, transmembrane domain and extracellular domain of LSECTin are shown below as SEQ ID No. 105, 106 and 107 respectively.

(LSECTin cytoplasmic domain)
SEQ ID No. 105
MDTTRYSKWGGSSEEVPGGPWGRWVHWSRRP
(LSECTin transmembrane domain)
SEQ ID No. 106
LFLALAVLVTTVLWAVILSIL
(LSECTin extracellular domain)
SEQ ID No. 107
LSKASTERAALLDGHDLLRTNASKQTAALGALKEEVGDCHSCCSGTQA
QLQTTRAELGEAQAKLMEQESALRELRERVTQGLAEAGRGREDVRTEL
FRALEAVRLQNNSCEPCPTSWLSFEGSCYFFSVPKTTWAAAQDHCADA
SAHLVIVGGLDEQGFLTRNTRGRGYWLGLRAVRHLGKVQGYQWVDGVS
LSFSHWNQGEPNDAWGRENCVMMLHTGLWNDAPCDSEKDGWICEKRHN
C

The effector immune cell of the present invention may comprise the LSECTin extracellular domain and optionally the LSECTin signal peptide and/or LSECTin transmembrane domain.

### FGL1

Fibrinogen-like protein 1 (FGL-1) is a protein that is structurally related to fibrinogen. It is an immune suppressive molecule that inhibits antigen-specific T-cell activation by acting as a major ligand of LAG3. FGL-1 is responsible for LAG3 T-cell inhibitory function and binds LAG3 independently from MHC class II (MHC-II).

The amino acid sequence of FGL1 is available from Uniprot, accession No. Q08830 and shown below as SEQ ID No. 108.

(FGL1 full sequence)
SEQ ID No. 108
MAKVFSFILVTTALTMGREISALEDCAQEQMRLRAQVRLLETRVKQQQV
KIKQLLQENEVQFLDKGDENTVIDLGSKRQYADCSEIFNDGYKLSGFYK
IKPLQSPAEFSVYCDMSDGGGWTVIQRRSDGSENFNRGWKDYENGFGNF
VQKHGEYWLGNKNLHFLTTQEDYTLKIDLADFEKNSRYAQYKNFKVGDE
KNFYELNIGEYSGTAGDSLAGNFHPEVQWWASHQRMKFSTWDRDHDNYE
GNCAEEDQSGWWFNRCHSANLNGVYYSGPYTAKTDNGIVWYTWHGWWYS
LKSVVMKIRPNDFIPNVI

The signal peptide of FGL1 is shown below as SEQ ID No. 109.

(FGL1 signal peptide)
SEQ ID No. 109
MAKVFSFILVTTALTMGREISA

The effector immune cell of the present invention may comprise FGL1 or the LAG-3 binding domain from FGL1 and optionally the FGL1 signal peptide.

The effector immune cell of the present invention may comprise a membrane-tethered version of FGL1 or a portion thereof. FGL1 may be tethered to the membrane using a transmembrane domain and optionally a spacer sequence and/or endodomain. For example, FGL1 or a portion thereof could be tethered to the membrane using the CD8 stalk spacer, transmembrane domain and truncated endodomain which has been previously described in WO2013/153391 for the sort-suicide gene RQR8.

### B7-H3

B7-H3, also known as CD276, is an immune checkpoint molecule expressed by some solid tumours and is involved in the regulation of T-cell-mediated immune response.

The amino acid sequence of B7-H3 is available from Uniprot, accession No. Q5ZPR3 and shown below as SEQ ID No. 110.

(B7-H3 full sequence)
SEQ ID No. 110
MLRRRGSPGMGVHVGAALGALWFCLTGALEVQVPEDPVVALVGTDATLC
CSFSPEPGFSLAQLNLIWQLTDTKQLVHSFAEGQDQGSAYANRTALFPD
LLAQGNASLRLQRVRVADEGSFTCFVSIRDFGSAAVSLQVAAPYSKPSM
TLEPNKDLRPGDTVTITCSSYQGYPEAEVFWQDGQGVPLTGNVTTSQMA
NEQGLFDVHSILRVVLGANGTYSCLVRNPVLQQDAHSSVTITPQRSPTG
AVEVQVPEDPWALVGTDATLRCSFSPEPGFSLAQLNLIWQLTDTKQLVH
SFTEGRDQGSAYANRTALFPDLLAQGNASLRLQRVRVADEGSFTCFVSI
RDFGSAAVSLQVAAPYSKPSMTLEPNKDLRPGDTVTITCSSYRGYPEAE
VFWQDGQGVPLTGNVTTSQMANEQGLFDVHSVLRVVLGANGTYSCLVRN
PVLQQDAHGSVTITGQPMTFPPEALWVTVGLSVCLIALLVALAFVCWRK
IKQSCEEENAGAEDQDGEGEGSKTALQPLKHSDSKEDDGQEIA

The signal peptide, extracellular domain and transmembrane domain of B7-H3 are shown below as SEQ ID No. 111, 112 and 113 respectively.

(B7-H3 signal peptide)
SEQ ID No. 111
MLRRRGSPGMGVHVGAALGALWFCLTGA
(B7-H3 extracellular domain)
SEQ ID No. 112
LEVQVPEDPVVALVGTDATLCCSFSPEPGFSLAQLNLIWQLTDTKQLV
HSFAEGQDQGSAYANRTALFPDLLAQGNASLRLQRVRVADEGSFTCFV
SIRDFGSAAVSLQVAAPYSKPSMTLEPNKDLRPGDTVTITCSSYQGYP
EAEVFWQDGQGVPLTGNVTTSQMANEQGLFDVHSILRVVLGANGTYSC
LVRNPVLQQDAHSSVTITPQRSPTGAVEVQVPEDPVVALVGTDATLRC
SFSPEPGFSLAQLNLIWQLTDTKQLVHSFTEGRDQGSAYANRTALFPD
LLAQGNASLRLQRVRVADEGSFTCFVSIRDFGSAAVSLQVAAPYSKPS
MTLEPNKDLRPGDTVTITCSSYRGYPEAEVFWQDGQGVPLTGNVTTSQ
MANEQGLFDVHSVLRVVLGANGTYSCLVRNPVLQQDAHGSVTITGQPM
TFPPEA
(B7-H3 transmembrane domain)
SEQ ID No. 113
LWWVTVGLSVCLIALLVALAFV

The effector immune cell of the present invention may comprise the B7-H3 extracellular domain and optionally the B7-H3 signal peptide and/or B7-H3 transmembrane domain.

### B7-H4

B7-H4, also known as V-set domain-containing T-cell activation inhibitor 1, is another member of the B7 family of co-stimulatory proteins which acts as an immune checkpoint molecule. B7-H4 negatively regulates T-cell-mediated immune response by inhibiting T-cell activation, proliferation, cytokine production and development of cytotoxicity. When expressed on the cell surface of tumour macrophages, B7-H4 plays an important role, together with regulatory T-cells (Treg), in the suppression of tumour-associated antigen-specific T-cell immunity.

The amino acid sequence of B7-H4 is available from Uniprot, accession No. Q7Z7D3 and shown below as SEQ ID No. 114.

(B7-H4 full sequence)
SEQ ID No. 114
MASLGQILFWSIISIIIILAGAIALIIGFGISGRHSITVTTVASAGN
IGEDGILSCTFEPDIKLSDIVIQWLKEGVLGLVHEFKEGKDELSEQD
EMFRGRTAVFADQVIVGNASLRLKNVQLTDAGTYKCYIITSKGKGNA
NLEYKTGAFSMPEVNVDYNASSETLRCEAPRWFPQPTVVWASQVDQG
ANFSEVSNTSFELNSENVTMKVVSVLYNVTINNTYSCMIENDIAKAT
GDIKVTESEIKRRSHLQLLNSKASLCVSSFFAISWALLPLSPYLMLK

The signal peptide, extracellular domain and transmembrane domain of B7-H4 are shown below as SEQ ID No. 115, 116 and 117 respectively.

(B7-H4 signal peptide)
SEQ ID No. 115
MASLGQILFWSIISIIIILAGAIA
(B7-H4 extracellular domain)
SEQ ID No. 116
LIIGFGISGRHSITVTTVASAGNIGEDGILSCTFEPDIKLSDIVIQWLK
EGVLGLVHEFKEGKDELSEQDEMFRGRTAVFADQVIVGNASLRLKNVQL
TDAGTYKCYIITSKGKGNANLEYKTGAFSMPEVNVDYNASSETLRCEAP
RWFPQPTVVWASQVDQGANFSEVSNTSFELNSENVTMKVVSVLYNVTIN
NTYSCMIENDIAKATGDIKVTESEIKRRSHLQLLNSKAS
(B7-H4 transmembrane domain)
SEQ ID No. 117
LCVSSFFAISWALLPLSPYLM

The effector immune cell of the present invention may comprise the B7-H4 extracellular domain and optionally the B7-H4 signal peptide and/or B7-H4 transmembrane domain.

The effector immune cell may express a protein comprising the extracellular domain of PD-L1, PD-L2, HVEM, CD155, VSIG-3, Galectin-9, HLA-G, CEACAM-1, LSECTin, FGL1, B7-H3 B7-H4 with the sequences shown above or a variant thereof, for example a variant having at least 80%, 90%, 95% or 99% amino acid identity, provided that the resultant protein molecule retains the capacity to bind an inhibitory immunoreceptor on the target immune cell and inhibit activation of the target immune cell.

### Membrane Localisation Domain

The effector immune may express a fusion protein comprising the extracellular domain of an immunoinhibitory molecule and a membrane localisation domain.

The membrane localisation domain may be any sequence which causes the fusion protein to be attached to or held in a position proximal to the plasma membrane.

The membrane localisation domain may be or comprise a sequence which causes the nascent polypeptide to be attached initially to the ER membrane. As membrane material “flows” from the ER to the Golgi and finally to the plasma membrane, the protein remain associated with the membrane at the end of the synthesis/translocation process.

The membrane localisation domain may, for example, comprise a transmembrane sequence, a stop transfer sequence, a GPI anchor or a myristoylation/prenylation/palmitoylation site.

Myristoylation is a lipidation modification where a myristoyl group, derived from myristic acid, is covalently attached by an amide bond to the alpha-amino group of an N-terminal glycine residue. Myristic acid is a 14-carbon saturated fatty acid also known as n-Tetradecanoic acid. The modification can be added either co-translationally or post-translationally. N-myristoyltransferase (NMT) catalyzes the myristic acid addition reaction in the cytoplasm of cells. Myristoylation causes membrane targeting of the protein to which it is attached, as the hydrophobic myristoyl group interacts with the phospholipids in the cell membrane.

The fusion protein may comprise a sequence capable of being myristoylated by a NMT enzyme. The fusion protein may comprise a myristoyl group when expressed in a cell.

The fusion protein may comprise a consensus sequence such as: NH2-G1-X2-X3-X4-S5-X6-X7-X8 which is recognised by NMT enzymes.

Palmitoylation is the covalent attachment of fatty acids, such as palmitic acid, to cysteine and less frequently to serine and threonine residues of proteins. Palmitoylation enhances the hydrophobicity of proteins and can be used to induce membrane association. In contrast to prenylation and myristoylation, palmitoylation is usually reversible (because the bond between palmitic acid and protein is often a thioester bond). The reverse reaction is catalysed by palmitoyl protein thioesterases.

In signal transduction via G protein, palmitoylation of the a subunit, prenylation of the γ subunit, and myristoylation is involved in tethering the G protein to the inner surface of the plasma membrane so that the G protein can interact with its receptor.

The fusion protein may comprise a sequence capable of being palmitoylated. The fusion protein may comprise additional fatty acids when expressed in a cell which causes membrane localisation.

Prenylation (also known as isoprenylation or lipidation) is the addition of hydrophobic molecules to a protein or chemical compound. Prenyl groups (3-methyl-but-2-en-1-yl) facilitate attachment to cell membranes, similar to lipid anchors like the GPI anchor.

Protein prenylation involves the transfer of either a farnesyl or a geranyl-geranyl moiety to C-terminal cysteine(s) of the target protein. There are three enzymes that carry out prenylation in the cell, farnesyl transferase, Caax protease and geranylgeranyl transferase I.

The fusion protein may comprise a sequence capable of being prenylated. The fusion protein may comprise one or more prenyl groups when expressed in a cell which causes membrane localisation.

### Cytoplasmic Domain

The fusion protein may comprise a cytoplasmic domain from a protein other than the immunoinhibitory molecule from which the extracellular domain was derived.

The cytoplasmic domain may stabilise the fusion protein. The cytoplasmic domain may, for example, be derived from CD19. the complete cytoplasmic domain of CD19 is shown below as SEQ ID No. 118. The fusion protein may comprise all, or a portion of this sequence. For example, the fusion protein may comprise the first 10, 15, 20 or 25 amino acids of the cytoplasmic portion of CD19. The fusion protein may comprise the first 19 amino acids of the cytoplasmic portion of CD19 and have the sequence shown as SEQ ID No. 119.

(CD19 endodomain)
SEQ ID No. 118
QRALVLRRKRKRMTDPTRRFFKVTPPPGSGPQNQYGNVLSLPTPTSGL
GRAQRWAAGLGGTAPSYGNPSSDVQADGALGSRSPPGVGPEEEEGEGY
EEPDSEEDSEFYENDSNLGQDQLSQDGSGYENPEDEPLGPEDEDSFSN
AESYENEDEELTQPVARTMDFLSPHGSAWDPSREATSLGSQSYEDMRG
ILYAAPQLRSIRGQPGPNHEEDADSYENMDNPDGPDPAWGGGGRMGTW
STR
(truncated CD19 endodomain)
SEQ ID No. 119
QRALVLRRKRKRMTDPTRR
CO-STIMULATORY ENDODOMAIN

The effector immune cell may express a fusion protein comprising the extracellular domain of an immunoinhibitory molecule and a co-stimulatory endodomain.

The co-stimulatory endodomain may be or comprise an endodomain selected from one of the following proteins: 0028, ICOS, CTLA4, 41BB, 0027, 0030, OX-40, TACI, GITR, 002 and 0040. The amino acid sequences for these endodomains are shown below as SEQ ID No. 120-130 respectively.

(CD28 endodomain)
SEQ ID No. 120
RSKRSRLLHSDYMNMTPRRPGPTRKHYQPYAPPRDFAAYRS
(ICOS endodomain)
SEQ ID No. 121
CWLTKKKYSSSVHDPNGEYMFMRAVNTAKKSRLTDVTL
(CTLA4 endodomain)
SEQ ID No. 122
AVSLSKMLKKRSPLTTGVYVKMPPTEPECEKQFQPYFIPIN
(41BB endodomain)
SEQ ID No. 123
KRGRKKLLYIFKQPFMRPVQTTQEEDGCSCRFPEEEEGGCEL
(CD27 endodomain)
SEQ ID No. 124
QRRKYRSNKGESPVEPAEPCHYSCPREEEGSTIPIQEDYRKPEPACSP
(CD30 endodomain)
SEQ ID No. 125
CHRRACRKRIRQKLHLCYPVQTSQPKLELVDSRPRRSSTQLRSGASVTE
PVAEERGLMSQPLMETCHSVGAAYLESLPLQDASPAGGPSSPRDLPEPR
VSTEHTNNKIEKIYIMKADTVIVGTVKAELPEGRGLAGPAEPELEEELE
ADHTPHYPEQETEPPLGSCSDVMLSVEEEGKEDPLPTAASGK
(OX-40 endodomain)
SEQ ID No. 126
ALYLLRRDQRLPPDAHKPPGGGSFRTPIQEEQADAHSTLAKI
(TACI endodomain)
SEQ ID No. 127
KKRGDPCSCQPRSRPRQSPAKSSQDHAMEAGSPVSTSPEPVETCSFCFP
ECRAPTQESAVTPGTPDPTCAGRWGCHTRTTVLQPCPHIPDSGLGIVCV
PAQEGGPGA
(GITR endodmain)
SEQ ID No. 128
QLGLHIWQLRSQCMWPRETQLLLEVPPSTEDARSCQFPEEERGERSAEE
KGRLGDLWV
(CD2 endodomain)
SEQ ID No. 129
KRKKQRSRRNDEELETRAHRVATEERGRKPHQIPASTPQNPATSQHPPP
PPGHRSQAPSHRPPPPGHRVQHQPQKRPPAPSGTQVHQQKGPPLPRPRV
QPKPPHGAAENSLSPSS
(CD40 endodomain)
SEQ ID No. 130
KKVAKKPTNKAPHPKQEPQEINFPDDLPGSNTAAPVQETLHGCQPVTQE
DGKESRISVQERQ

The fusion protein may comprise a combination of endodomains, such as CD28 and OX-40 or CD28 and 4-1BB.

The fusion protein may comprise a variant of one of the sequences shown as SEQ ID No. 120-130, for example a variant having at least 80%, 90%, 95% or 99% amino acid identity, provided that the resultant sequence retains the capacity to provide a proliferation and/or survival signal to the effector immune cell.

### Nucleic Acid Sequence

The present invention also provides a nucleic acid sequence encoding a fusion protein which comprises the extracellular domain of an immunoinhibitory molecule, together with:
(a) a heterogeneous transmembrane domain (i.e. not derived from the immunoinhibitory molecule); and/or
(b) an heterogeneous endodomain (i.e. not derived from the immunoinhibitory molecule).

The endodomain may comprise one or more co-stimulatory domains as defined above.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

### Nucleic Acid Construct

The present invention also provides a nucleic acid construct which comprises: (i) a first nucleic acid sequence which encodes a cell surface receptor or part of a cell surface receptor complex as defined above; and
(ii) a second nucleic acid sequence which, when expressed in a cell, confers on that cell resistance to an immunosuppressant; and/or
(iii) a third nucleic acid sequence which encodes an immunoinhibitory molecule or a fusion protein comprising the extracellular domain of an immunoinhibitory molecule.

The first nucleic acid sequence may encode:
(a) a chimeric antigen receptor (CAR); and/or
(b) an engineered polypeptide which comprises the ectodomain from an MHC class I polypeptide or the ectodomain from an MHC class II polypeptide linked to an intracellular signalling domain; or β-2 microglobulin linked to an intracellular signalling domain;
(c) an engineered polypeptide which comprises an MHC class I polypeptide or MHC class II polypeptide or β-2 microglobulin linked to linked to a component of the CD3/TCR complex, such as CD3-zeta, CD3-epsilon, CD3-gamma or CD3-delta;
(d) an engineered polypeptide which comprises a binding domain, such as an antibody-like binding domain, which binds to an MHC class I polypeptide, an MHC class II polypeptide or β-2 microglobulin, linked to an intracellular signalling domain;
(f) an engineered polypeptide which comprises CD79α or CD79β linked to an intracellular signalling domain;
(g) an engineered polypeptide which comprises the MHC class II-binding domain of CD4 linked to an intracellular signalling domain; or the MHC class I-binding domain of CD8 linked to an intracellular signalling domain; or
(e) a bispecific polypeptide which comprises: (i) a first binding domain which binds to MHC class I polypeptide; an MHC class II polypeptide; β-2 microglobulin; and (ii) a second binding domain which binds to a component of the TCR/CD3 complex.

The second nucleic acid sequence may encode:
(e) variant calcineurin with increased resistance to one or more calcineurin inhibitors than wild-type calcineurin; and/or
(f) dominant negative CSK.

The third nucleic acid sequence may encode:
(g) an immunoinhibitory molecule or a fusion protein comprising the extracellular domain of an immunoinhibitory molecule.

In a first embodiment, the present invention provides a nucleic acid construct which comprises:
(i) a first nucleic acid sequence which encodes CAR which specifically binds TRBC1 or TRBC2; and
(ii) a second nucleic acid sequence encoding variant calcineurin with increased resistance to one or more calcineurin inhibitors than wild-type calcineurin, and/or dominant negative CSK; and/or
(iii) a third nucleic acid sequence which encodes an immunoinhibitory molecule or a fusion protein comprising the extracellular domain of an immunoinhibitory molecule.

In a second embodiment, the present invention provides a nucleic acid construct which comprises:
(i) a first nucleic acid sequence which encodes β-2 microglobulin linked to an intracellular signalling domain; and
(ii) a second nucleic acid sequence encoding variant calcineurin with increased resistance to one or more calcineurin inhibitors than wild-type calcineurin, and/or dominant negative CSK; and/or
(iii) a third nucleic acid sequence which encodes an immunoinhibitory molecule or a fusion protein comprising the extracellular domain of an immunoinhibitory molecule.

The nucleic acid construct of the second embodiment may also comprise a nucleic acid sequence encoding a CAR.

The nucleic acids may be in any order in the construct. Nucleic acids encoding discrete polypeptides may be separated by a co-expression site enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No.132 (RAEGRGSLLTCGDVEENPGP).

### Vector

The present invention also provides a vector, or kit of vectors, which comprises one or more nucleic acid sequence(s) or nucleic acid construct(s) according to the invention. Such a vector may be used to introduce the nucleic acid sequence(s) into a host cell so that it expresses a cell surface receptor or receptor complex, together with one or more proteins which confer a selective advantage of the host cell (i.e. effector immune cell) than a target immune cell.

A kit of vectors may comprise:
(i) a first vector comprising a nucleic acid sequence which encodes a cell surface receptor or part of a cell surface receptor complex; and
(ii) a second vector comprising a nucleic acid sequence which, when expressed in a cell, confers on that cell resistance to an immunosuppressant; and/or
(iii) a third vector comprising a nucleic acid sequence which encodes an immunoinhibitory molecule or a fusion protein comprising the extracellular domain of an immunoinhibitory molecule.

In a first embodiment, the present invention provides a kit of vectors which comprises:
(i) a first vector comprising a nucleic acid sequence which encodes CAR which specifically binds TRBC1 or TRBC2; and
(ii) a second vector comprising a nucleic acid sequence encoding variant calcineurin with increased resistance to one or more calcineurin inhibitors than wild-type calcineurin, and/or dominant negative CSK; and/or
(iii) a third vector comprising a nucleic acid sequence which encodes an immunoinhibitory molecule or a fusion protein comprising the extracellular domain of an immunoinhibitory molecule.

In a second embodiment, the present invention provides a kit of vectors which comprises:
(i) a first vector comprising a nucleic acid sequence which encodes β-2 microglobulin linked to an intracellular signalling domain; and
(ii) a second vector comprising a nucleic acid sequence encoding variant calcineurin with increased resistance to one or more calcineurin inhibitors than wild-type calcineurin, and/or dominant negative CSK; and/or
(iii) a third vector comprising a nucleic acid sequence which encodes an immunoinhibitory molecule or a fusion protein comprising the extracellular domain of an immunoinhibitory molecule.

The kit of vectors of the second embodiment may also comprise vector comprising a nucleic acid sequence encoding a CAR.

The vector may, for example, be a plasmid or a viral vector, such as a retroviral vector or a lentiviral vector, or a transposon based vector or synthetic mRNA.

The vector may be capable of transfecting or transducing a cell, such as a T cell or a NK cell.

### Cell

The present invention provides an effector immune cell.

The cell may comprise a nucleic acid sequence, a nucleic acid construct or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells according to the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cells may be activated and/or expanded prior to being transduced with nucleic acid encoding the molecules providing the chimeric polypeptide according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s), nucleic acid construct(s) or vector(s) of the invention.

The cells may then by purified, for example, selected on the basis of expression of one or more heterologous nucleic acid sequences.

The effector immune cell is capable of recognising and killing a target immune cell. The target immune cell may be a cytolytic immune cell such as a T-cell or NK cell as defined above.

### Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The cell-containing sample may be isolated from a subject or from other sources, as described above.

The present invention also provides a method for treating a disease in a subject, which comprises the following steps:
(i) administering a pharmaceutical composition to a subject, which pharmaceutical composition comprises a plurality of effector immune cells engineered to be resistant to an immunosuppressant; and
(ii) administering the immunosuppressant to the subject.

The effector immune cells may express variant calcineurin engineered to be resistant to one or more calcineurin inhibitors, for example:
calcineurin A comprising mutations T351 E and L354A with reference to the shown as SEQ ID No. 65;
calcineurin A comprising mutations V314R and Y341F and with reference to shown as SEQ ID No. 65; or
calcineurin B comprising mutation L124T and K-125-LA-Ins with reference to shown as SEQ ID No. 66.

Step (ii) may involve administering cyclosporin and/or tacrolimus to the cells or to the patient.

The effector cells may express dnCSK and step (ii) may involve administering any immunosuppressant to the subject, for example rapamycin.

The present invention provides a cell of the present invention for use in treating and/or preventing a disease.

The invention also relates to the use of a cell of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The disease may be a plasma cell disorder such as plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

The effector immune cells of the present invention are capable of killing target immune cells, which may be cancer cells or normal immune cells which are reactive against the effector immune cell.

The present invention also provides a method for depleting alloreactive immune cells from a population of immune cells, which comprises the step of contacting the population of immune cells with a plurality of effector immune cells having an engineered MHC class I or an MHC class II complex as defined above.

The present invention also provides a method for treating or preventing graft rejection following allotransplantation, which comprises the step of administering a plurality of effector immune cells derived from the donor subject to the recipient subject for the allotransplant, the plurality of effector immune cells expressing an engineered MHC class I or an MHC class II complex as defined above.

The effector immune cells could be administered to the patient before, after or at the same time as the transplant. For example, for an organ transplant, effector T cells from the organ donor expressing an engineered MHC class I or an MHC class II complex as defined above could be infused into a recipient prior to transplant to eliminate alloreactive T-cells that could mediate graft rejection. Alternatively, in the case of HSCT, recipient T-cells expressing an engineered MHC class I or an MHC class II complex as defined above can be cultured with the stem cell graft prior to infusion to eliminate donor alloreactive T-cells that could attack host tissues.

There is also provided a method for treating or preventing graft versus host disease (GVHD) associated with allotransplantation, which comprises the step of contacting the allotransplant with administering a plurality of effector immune cells having an engineered MHC class I or an MHC class II complex as defined above.

The allotransplantation may involve adoptive transfer of allogeneic immune cells.

There is also provided an allotransplant which has been depleted of alloreactive immune cells by a method of the invention. There is also provided an allotransplant which comprises effector immune cells of the invention.

There is also provided effector immune cells of the invention for use in:
depleting alloreactive immune cells from a population of immune cells;
treating or preventing graft rejection following allotransplantation; or
treating or preventing graft versus host disease (GVHD) associated with allotransplantation.

There is also provided the use of effector immune cells of the invention in the manufacture of a pharmaceutical composition for:
depleting alloreactive immune cells from a population of immune cells;
treating or preventing graft rejection following allotransplantation; or
treating or preventing graft versus host disease (GVHD) associated with allotransplantation.

The invention will now be further described by way of Examples, which are meant to serve to assist one of ordinary skill in the art in carrying out the invention and are not intended in any way to limit the scope of the invention.

### EXAMPLES

### Example 1—Creation of a Model System Showing “Reverse” Killing of TRBC1-Binding CAR-T Cells by Target T Cells

WO2015/132598 describes a CAR which specifically bind TCR beta constant region 1 (TRBC1) which comprises the VH and VL domains shown as SEQ ID No. 7 and 8 respectively.

A truncated version of this CAR was created which lacks the signalling domain, named dJOVI; dJOVI binds TRBC1 on target cells but is unable to trigger T cell activation and killing. PBMCs were transduced with a vector expressing dJOVI or the full length CAR (JOVI) together with the sort-suicide gene RQR8 which is described in WO2013/153391. JOVI- or dJOVI-transduced PBMCs were co-cultured with TRBC1+ target PBMCs at a 1:2 effector:target ratio and live transduced (RQR8+) T cells were enumerated after 24 h of co-culture. The results are shown in FIG. 12. Killing of the effector cells by the TRBC1+ target cells was observed to a greater extent on dJOVI-transduced PBMCs, indicating that the binding of JOVI to TRBC1 on the targets is enough to trigger target T cell activation and cause reverse killing of the effector cells.

### Example 2—Expression of PDL1 or PDL2 by a TRBC1-Bindinq CAR-T Cell Reduces Reverse Killing of the Effector Cells

In order to investigate the effect of engineering the CAR-T cell to transmit inhibitory immune signals on reverse killing by target T cells, PBMCs were transduced to express JOVI− or dJOVI− together with a truncated version of PD-L1 or PDL2 which lacks the cytoplasmic domain (dPDL1 and dPDL2). For this assay, TRBC1+ target PBMCs were transduced to express full length PD1.

A co-culture with JOVI- or dJOVI-transduced PBMCs expressing dPDL1 or dPDL2 together with RQR8; and TRBC1+ target PBMCs expressing the PD1, was setup at a 1:1 effector:target ratio. Live transduced (RQR8+) T cells were enumerated after 72 h of co-culture and each condition normalized to its respective JOVI (or dJOVI) co-culture. The results are shown in FIG. 13. An increase in the number of recovered transduced cells was observed when dPDL1 or dPDL2 were expressed on the CAR compared to the CAR alone.

### Example 3—Expression of a Calcineurin Mutant by TRBC1-Bindinq CAR-T Cells Reduces Reverse Killing of the Effector Cells in the Present of a Calcineurin Inhibitor

A co-culture of JOVI-RQR8 transduced PBMCs expressing calcineurin mutants with TRBC1+ target PBMCs is setup at a 1:1 and 1:4 effector:target ratio. Different concentrations of calcineurin inhibitors are added to the co-culture. Live transduced (RQR8+) T cells are enumerated after 72 h of co-culture by flow cytometry and each condition normalized to the co-culture where no inhibitor is added.

### Example 4—Expression of dnCSK by TRBC1-Bindinq CAR-T Cell Reduces Reverse Killing of the Effector Cells in the Presence of an Immunosuppressant

A co-culture of JOVI-RQR8 transduced PBMCs expressing dnCSK with TRBC1+ target PBMCs is setup at a 1:1 and 1:4 effector:target ratio. Different concentrations of an immunosuppressant were added to the co-culture. Live transduced (RQR8+) T cells are enumerated after 72 h of co-culture by flow cytometry and each condition normalized to the co-culture where no immunosuppressant is added.

### Example 5—Expression of PDL1 or PDL2 by β2m-CD3ζ Expressing Effector T Cells Reduces Reverse Killing by the Target Cells

In order to investigate the effect of engineering the CAR-T cell to transmit inhibitory immune signals on reverse killing by target T cells with the addition of an antirejection kill response; PBMCs are transduced to express JOVI-, dJOVI- or an irrelevant CAR together with a truncated version of PD-L1 or PDL2 which lacks the cytoplasmic domain (dPDL1 and dPDL2) and a fusion protein consisting of B2M tethered to CD3zeta (β2m-CD3ζ). For this assay, TRBC1+ target PBMCs are transduced to express full length PD1 in the presence or absence of superantigens (SAgs) to ligate the armed MHC to the TCR. Superantigens are not processed intracellularly. Instead, they bind class II MHC molecules as intact macromolecules and bind outside of the peptide-antigen binding groove. SAgs are molecules that indiscriminately stimulate up to 20% of all T cells (normal response to antigen stimulates only 0.01% of T cells).

A co-culture with JOVI- or dJOVI- or an irrelevant CAR transduced PBMCs expressing dPDL1 or dPDL2 together with β2m-CD3ζ; and TRBC1+ target PBMCs expressing the PD1 plus a SAg, is setup at a 1:1 effector:target ratio. Live transduced T cells are enumerated after 72 h of co-culture and each condition normalized to its respective JOVI (or dJOVI) co-culture.

### Example 6—Expression of a Calcineurin Mutant by β2m-CD3ζ Expressing T Cells Reduces Reverse Killing of the Effector Cells in the Present of a Calcineurin Inhibitor

A co-culture of transduced PBMCs with a vector encoding a CAR (JOVI, dJOVI or an irrelevant CAR), β2m-CD3ζ and calcineurin mutants with TRBC1+ target PBMCs is setup at a 1:1 and 1:4 effector:target ratio. Different concentrations of calcineurin inhibitors and SAgs are added to the co-culture. Live transduced T cells were enumerated after 72 h of co-culture by flow cytometry and each condition normalized to the co-culture where no inhibitor or SAgs are added.

### Example 7—Expression of dnCSK by β2m-CD3ζ Expressing T Cells Reduces Reverse Killing of the Effector Cells in the Presence of an Immunosuppressant

A co-culture of transduced PBMCs with a vector encoding a CAR (JOVI, dJOVI or an irrelevant CAR), β2m-CD3ζ and dnCSK with TRBC1+ target PBMCs is setup at a 1:1 and 1:4 effector:target ratio. Different concentrations of immunosuppressants and SAgs are added to the co-culture. Live transduced T cells are enumerated after 72 h of co-culture by flow cytometry and each condition normalized to the co-culture where no immunosuppressants or SAgs are added.

### Example 8—Expression of a Calcineurin Mutant by TRBC2-Binding CAR-T Cells Gives Resistance to Inhibition of Proliferation by a Calcineurin Inhibitor

PBMCs were transduced with a vector expressing a CAR together with the sort-suicide gene RQR8 which is described in WO2013/153391. The CARs tested are summarised below:

CD19 CAR: A second generation CAR having an antigen binding domain derived from Fmc63, a hinge spacer and a 41BB/CD3z endodomain
TRBC1 CAR: A second generation CAR having an antigen binding domain as described in WO2018/224844, a hinge spacer and a 41BB/CD3z endodomain
TRBC2 CAR: A second generation CAR having an antigen binding domain as described in WO2020/089644, a CD8 stalk spacer and a CD28/CD3z endodomain

One population of cells were transduced with a tricistronic vector expressing RQR8, the TRBC2 CAR and the CnB30 calcineurin mutant module described above having SEQ ID No. 131.

Transduced cells were co-cultured with one of the following target cell types:

Jurkat TRBC1: wild-type Jurkat cells which express TRBC1
Jurkat KO: Jurkat cells engineered to lack TRBC1 expression
Jurkat TRBC2: Jurkat cells in which the TRBC1 gene is replaced by the TRBC2 gene using CRISPR-Cas9 technology so that the expression of TRBC2 is the same as that of TRBC1 on the wild-type cell.

Cells were co-cultures for 96 hours at a 1:4 E:T ratio in the presence or absence of 20 ng/ml of Tacrolimus. Transduced effector cells were identified based on their expression of RQR8 and their proliferation analysed using cell-trace violet (CTV) dilution. The results are shown in FIGS. 16 and 17. As expected, in the absence of Tacrolimus, cells expressing the TRBC1 CAR showed an increase in the percentage and number of proliferating cells following co-culture with TRBC1-expressing target cells; and cells expressing the TRBC2 CAR showed an increase in the percentage and number of proliferating cells following co-culture with TRBC2-expressing target cells (FIG. 16). In the presence of Tacrolimus, proliferation of CAR-T cells is inhibited as can be seen by comparing “TRBC2 CAR” in FIG. 16B (without Tacrolimus) and in FIG. 17B (with Tacrolimus). Only the cells co-expressing the TRBC2 CAR and the CnB30 calcineurin mutant showed an increase in the absolute number (FIG. 17B) of transduced effector cells following co-culture with TRBC2-expressing targets. This population also showed the highest percentage of transduced proliferating cells (FIG. 17A).

Proliferation analysis was also calculated on single/live/CellTrace Violet-positive cells using the FlowJo proliferation tool using CD19 CAR as the negative control. The cell number in each division was plotted for each CAR+target combination described above and the results are shown in FIGS. 18 (without Tacrolimus) and 19 (with tacrolimus). The results for two separate donors are also shown in the histogram plots of FIG. 20. Again, in the presence of Tacrolimus, only the cells co-expressing the TRBC2 CAR and the CnB30 calcineurin mutant showed an increase in proliferation of effector cells following co-culture with TRBC2-expressing targets (FIG. 19, bottom graph; and FIG. 20).

In a similar study, cells transduced to express either TRBC2 CAR alone or TRBC2 CAR in combination with a calcineurin mutant (CnB30) were co-cultured for 4 days with TRBC2+ positive targets in the presence or absence of 20 ng/ml of Tacrolimus. The number CAR-expressing cells after 4 days' co-culture is shown in FIG. 21. The only cell population with a high number of cells expressing TRBC2 CAR after co-culture in the presence of Tacrolimus were the cells co-expressing TRBC2 CAR with the calcineurin mutant (TRBC2 CAR+CnB30).

FIG. 22 shows the percentage of RQR8-expressing cells. While the percentage of CD19-expressing cells stayed constant, the percentage of cells expressing TRBC2 CAR increased following co-culture with TRBC2+ targets in the absence of Tacrolimus. This was true for cells expressing TRBC2 CAR alone or co-expressing TRBC2 CAR in combination with the calcineurin mutant. In the presence of Tacrolimus, the percentage of RQR8+ cells expressing TRBC2 CAR alone was reduced, showing that Tacrolimus inhibits proliferation of these cells. By contrast, the percentage of RQR8+ cells co-expressing TRBC2 CAR/CnB30 was the same as in the co-culture without Tacrolimus, indicating that these cells show resistance to calcineurin inhibition.

### Example 9—Investigating the Effect of Expression of a Calcineurin Mutant by Anti TRBC2-Expressing Cells on Reverse Killing by TRBC2 Expressing Target Cells

PBMCs from healthy donors were magnetically sorted into TRBC1+ and TRBC2+ fractions. After 2 days of activation, the TRBC1+ fractions were transduced with retroviral vectors expressing RQR8 and either CD19 or TRBC2 CAR as described above. One population of cells were transduced with a tricistronic vector expressing RQR8, the TRBC2 CAR and the CnB30 calcineurin mutant module described above having SEQ ID No. 131.

Cells were left untreated or treated with 20 ng/ml Tacrolimus at day 3 post-transduction and expanded for further 4 days under these conditions. Seven days post-transduction, killing assays were setup at effector:target ratios of 1:1 and 1:4 and the non-transduced TRBC2+ fraction was labelled with Cell Trace Violet and used as an autologous target.

Killing was assessed by flow cytometry 72 h later and supernatant from the co-cultures collected and analysed for IFNγ and IL-2 production. Transduced effector cells were identified based on their expression of RQR8. The results are shown in FIGS. 23 to 26.

Following expansion and co-culture in the presence of tacrolimus, improved target cell killing was observed with the effector cell population which co-expressed the TRBC2 CAR with the CnB30 calcineurin mutant compared with the effector cell population expressing the TRBC2 CAR alone (FIG. 23). The effect was particularly noticeable when cells were co-cultured at a 1:4 E:T ratio.

Following 72-hour co-culture with TRBC2-expressing PBMC in the absence of tacrolimus at a 1:1 ratio, some anti-TRBC2 CAR-expressing cells were detectable (FIG. 24, first graph). However, when CAR-expressing cells were co-cultured with TRBC2-expressing PBMC at a 1:4 ratio, the CAR T-cell count was close to zero (FIG. 24, second graph) presumably due to reverse killing of the CAR-expressing cells by the target cells.

Following expansion and co-culture in the presence of tacrolimus, however, the effector cell population which co-expressed the TRBC2 CAR with the CnB30 calcineurin mutant showed some survival/proliferation even following co-culture at a 1:4 ratio. At a 1:1 co-culture ratio, the effector cell population which co-expressed the TRBC2 CAR with the CnB30 calcineurin mutant showed much greater survival/proliferation than the effector cell population expressing the TRBC2 CAR alone (FIG. 24, third and fourth graphs).

The cell population which co-expressed the TRBC2 CAR with the CnB30 calcineurin mutant also showed increased T-cell activation in terms of cytokine release following expansion and co-culture in the presence of tacrolimus than the cell population expressing the TRBC2 CAR alone. This is true for both IFNγ (FIG. 25) and IL-2 (FIG. 26).

Taken together, these data indicate that expression of a calcineurin mutant by CAR-expressing cells gives the effector cells an advantage over the target T cells and prevents reverse killing by the target cells.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An effector immune cell which expresses a cell surface receptor or receptor complex which specifically binds an antigen recognition receptor of a target immune cell; which effector immune cell is engineered to be resistant to one or more calcineurin inhibitors.

2-3. (canceled)

4. An effector immune cell according to claim 3, which expresses:
calcineurin A comprising mutations T351 E and L354A with reference to the shown as SEQ ID No. 65;
calcineurin A comprising mutations V314R and Y341F and with reference to shown as SEQ ID No. 65; or
calcineurin B comprising mutation L124T and K-125-LA-Ins with reference to shown as SEQ ID No. 66.

5-16. (canceled)

17. An effector immune cell according to claim 1, which expresses a cell surface receptor complex which comprises: an MHC class I polypeptide; an MHC class II polypeptide; or β-2 microglobulin, linked to an intracellular signalling domain.

18-24. (canceled)

25. A nucleic acid construct which comprises:
(i) a first nucleic acid sequence which encodes a cell surface receptor or part of a cell surface receptor complex which specifically binds an antigen recognition receptor of a target immune cell; and
(ii) a second nucleic acid sequence which, when expressed in a cell, confers on that cell resistance to one or more calcineurin inhibitors.

26. A vector comprising a nucleic acid construct according to claim 25.

27. A kit of vectors comprising:
(i) a first vector comprising a first nucleic acid sequence which encodes a cell surface receptor or part of a cell surface receptor complex which specifically binds an antigen recognition receptor of a target immune cell; and
(ii) a second vector comprising a second nucleic acid sequence which, when expressed in a cell, confers on that cell resistance to one or more calcineurin inhibitors.

28. A pharmaceutical composition comprising a plurality of effector immune cells according to claim 1.

29. (canceled)

30. A method for treating a disease, which comprises the step of administering a pharmaceutical composition according to claim 28 to a subject.

31. A method according to claim 30, which comprises the following steps:
(i) administering a pharmaceutical composition to a subject, which pharmaceutical composition comprises a plurality of effector immune cells which express a cell surface receptor or receptor complex which specifically binds an antigen recognition receptor of a target immune cell, and which are engineered to be resistant to one or more calcineurin inhibitors; and
(ii) administering the immunosuppressant to the subject.

32. (canceled)

33. A method according to claim 30, wherein the disease is cancer.

34. A method for making an effector immune cell according to claim 1, which comprises the step of introducing into the cell:
(i) a first nucleic acid sequence which encodes a cell surface receptor or part of a cell surface receptor complex which specifically binds an antigen recognition receptor of a target immune cell, and
(ii) a second nucleic acid sequence which, when expressed in a cell, confers on that cell resistance to one or more calcineurin inhibitors.

35-39. (canceled)
